(19) United States
(12) Patent Application Publication
(10) Pub. No.: US 2021/0079066 A1
(43) Pub. Date: Mar. 18, 2021

(54) RADICALLY DIVERSE HUMAN ANTIBODY LIBRARY

(71) Applicant: Distributed Bio, Inc., South San Francisco, CA (US)

(72) Inventors: Jacob Glanville, San Francisco, CA (US); David Maurer, San Francisco, CA (US)

(21) Appl. No.: 16/899,519

(22) Filed: Jun. 11, 2020

Related U.S. Application Data
(63) Continuation of application No. PCT/US2018/066318, filed on Dec. 18, 2018.
(60) Provisional application No. 62/607,199, filed on Dec. 18, 2017.
(60) Provisional application No. 62/753,754, filed on Oct. 31, 2018.

(51) Int. Cl. C07K 16/00; C40B 40/10; C07K 16/28

(52) U.S. Cl. CPC C07K 16/005; C40B 40/10; C07K 2317/567; C07K 2317/10; C07K 2317/565; C07K 16/2818

(57) ABSTRACT

Disclosed is an antibody library comprising a plurality of antibodies with non-naturally occurring combinations of complementary determining regions from memory and naïve B-cells naturally occurring in humans, and wherein the antibody library comprises a high number of functional and non-redundant antibodies. Further disclosed are methods of preparing antibody libraries with a high level of functional diversity.

### CROSS-REFERENCE

This application is a continuation application of International Patent Application No. PCT/US2018/066318, filed Dec. 18, 2018, which claims the benefit of U.S. Provisional Application Nos. 62/607,199, filed Dec. 18, 2017 and 62/753,754, filed Oct. 31, 2018, each of which is entirely incorporated herein by reference.

### BACKGROUND OF THE INVENTION

Monoclonal antibodies (mAbs) are useful as therapeutics, research tools, and in diagnostics, but finding an antibody with affinity to a desired target can be challenging. Antibody libraries provide an efficient tool for screening a large amount of antibodies against a target compound. Such libraries are typically based on the rearrangement of naturally occurring variable genes or introduce synthetic diversity into the antibody sequence. However, natural antibody libraries often have extremely limited diversity while synthetic libraries can be plagued with non-functional sequences. Therefore, a need exists for development of antibody libraries with a high degree of functional diversity.

### SUMMARY OF THE INVENTION

Provided herein is an antibody library, which comprises a plurality of antibodies. The plurality of antibodies can comprise a VH domain comprising a VH-CDR1 sequence, a VH-CDR2 sequence, a VH-CDR3 sequence and a VL domain comprising a VL-CDR1 sequence, a VL-CDR2 sequence, a VL-CDR3 sequence. At least one of the VH-CDR3 sequence and the VL-CDR3 sequence can be derived from a naïve B-cell. In some embodiments, if only one of the VH-CDR3 sequence and the VL-CDR3 sequence is derived from the naïve B-cell, then the VH-CDR3 sequence or VL-CDR3 sequence not derived from the naïve B-cell is derived from a memory cell. The VH-CDR1 sequence, VH-CDR2 sequence, VL-CDR1 sequence, and VL-CDR2 sequence may be derived from a memory B cell. In some embodiments, the at least one of the VH-CDR3 sequence and the VL-CDR3 sequence derived from a naïve B-cell is a naturally occurring sequence. In some embodiments, the VH-CDR3 sequence or VL-CDR3 sequence derived from a memory cell is a naturally occurring sequence. In some embodiments, the VH-CDR1 sequence, VH-CDR2 sequence, VL-CDR1 sequence, and VL-CDR2 sequence derived from a memory B cell are naturally occurring sequences. In some embodiments, the at least one of the VH-CDR3 sequence and the VL-CDR3 sequence derived from a naïve B-cell comprises at least 80% sequence homology to a naturally occurring sequence. In some embodiments, the VH-CDR3 sequence or VL-CDR3 sequence derived from a memory cell comprises at least 80% sequence homology to a naturally occurring sequence. In some embodiments, the VH-CDR1 sequence, VH-CDR2 sequence, VL-CDR1 sequence, and VL-CDR2 sequence derived from a memory B cell comprise at least 80% sequence homology to a naturally occurring sequence. In some embodiments, the VL domain is a VK domain or a Vλ domain. In some embodiments, the naïve B-cell is a CD27−/IgM+ B-cell or a CD27−/IgD+ B-cell. In some embodiments, the memory B-cell is selected from the group consisting of a CD27+/IgG+ B-cell, a CD27+/IgM+ B-cell, an IgA+ B-cell, and a combination thereof. In some embodiments, the naïve B-cell and memory B-cell are from a sample comprising a plurality of naïve B-cells and memory B-cells sampled from a plurality of individuals. In some embodiments, the plurality of individuals is at least 50 individuals. In some embodiments, the plurality of antibodies are expressed on the surface of a plurality of phages. In some embodiments, the plurality of phages are bacteriophages or phagemids. In some embodiments, each phage of the plurality of phages comprises a nucleic acid sequence encoding: i) an antibody of the plurality of antibodies, and ii) a gene encoding a phage coat protein. In some embodiments, the phage coat protein is protein gIII. In some embodiments, expression of the nucleic acid sequence of each phage produces an antibody fused to a phage coat protein. In some embodiments, the VH domain further comprises framework regions selected from the group consisting of IGHJ4, IGHV1-46, IGHV1-69, IGHV3-15, and IGHV3-23. In some embodiments, the VL domain further comprises framework regions selected from the group consisting of IGKV1-39, IGKV2-28, IGKV3-15, and IGKV4-1. In some embodiments, the plurality of antibodies comprises at least 7.6×10¹⁰ antibodies. In some embodiments, at least 95% of the plurality of antibodies are functional.

Also provided herein are methods of preparing an antibody library, comprising a) obtaining sequence information for a plurality of VH-CDR3 and VL-CDR3 sequences from a pool of naïve B-cells and sequence information for a plurality of VH-CDR1, VH-CDR2, VH-CDR3, VL-CDR1, VL-CDR2, and VL-CDR3 sequences from a pool of memory B-cells; b) assembling a plurality of variable light (VL) domain sequences, each VL domain sequence comprising: a VL-CDR1 sequence derived from the sequence information from memory B-cells determined in step a., a VL-CDR2 sequence derived from the sequence information from memory B-cells determined in step a., and a VL-CDR3 sequence derived from the sequence information from memory B-cells or naïve B-cells determined in step a., c) assembling a plurality of first nucleic acid sequences encoding a plurality of first antibodies, each first antibody comprising a variable light (VL) domain sequence assembled in step b. and a single fixed heavy chain sequence; d) inserting the plurality of first nucleic acid sequences into a plurality of phages; e) expressing the plurality of first antibodies on the surface of the plurality of phages; f) applying at least one selective pressure to the plurality of phages to produce a subset of phages comprising a subset of first nucleic acid sequences; g) assembling a plurality of a variable heavy (VH) domain sequences, each VH domain sequence comprising: a VH-CDR1 sequence derived from the sequence information from memory B-cells determined in step a., a VH-CDR2 sequence derived from the sequence information from memory B-cells determined in step a., and a VH-CDR3 sequence derived from the sequence information from memory B-cells or naïve B-cells determined in step a., wherein at least one of the VH-CDR3 sequence and the VL-CDR3 sequence is derived from the sequence information from naïve B-cells; h) replacing the single fixed heavy chain sequences from the subset of first nucleic acid sequences with the plurality of VH domain sequences assembled in step g. to produce a plurality of second nucleic acid sequences, each second nucleic acid sequence comprising a variable light (VL) domain sequence assembled in step b. and a variable heavy (VH) domain sequence assembled in step g., wherein the plurality of second nucleic acid sequences encodes a plurality of second antibodies; and i) transforming a plurality of microbes with the plurality of phages to produce a plurality of transformants. In some embodiments, the pool of naïve B-cells comprises less than 5% of cells not of naïve B-cell origin. In some embodiments, the pool of memory B-cells comprises less than 5% of cells not of memory B-cell origin. In some embodiments, the at least one of the VH-CDR3 sequence and the VL-CDR3 sequence derived from a naïve B-cell is a naturally occurring sequence. In some embodiments, the VH-CDR3 sequence or VL-CDR3 sequence derived from a memory cell is a naturally occurring sequence. In some embodiments, the VH-CDR1 sequence, VH-CDR2 sequence, VL-CDR1 sequence, and VL-CDR2 sequence derived from a memory B cell are naturally occurring sequences. In some embodiments, the at least one of the VH-CDR3 sequence and the VL-CDR3 sequence derived from a naïve B-cell comprises at least 80% sequence homology to a naturally occurring sequence. In some embodiments, the VH-CDR3 sequence or VL-CDR3 sequence derived from a memory cell comprises at least 80% sequence homology to a naturally occurring sequence. In some embodiments, the VH-CDR1 sequence, VH-CDR2 sequence, VL-CDR1 sequence, and VL-CDR2 sequence derived from a memory B cell comprise at least 80% sequence homology to a naturally occurring sequence. In some embodiments, the pool of naïve B-cells, the pool of memory cells, or the combination thereof is obtained from a plurality of individuals. In some embodiments, the plurality of individuals is at least 50 individuals. In some embodiments, the method further comprises sorting the naïve B-cells and memory B-cells in a sample to produce the pool of naïve B-cells and the pool of memory B-cells prior to obtaining the sequence information. In some embodiments, sorting the naïve B-cells and the memory B-cells comprises the use of flow cytometry. In some embodiments, the flow cytometry is fluorescence-activated cell sorting (FACS). In some embodiments, the method further comprises extracting nucleic acid from the naïve B-cells and the memory B-cells. In some embodiments, the nucleic acid is DNA. In some embodiments, the nucleic acid is mRNA. In some embodiments, the method further comprises reverse transcribing the mRNA to complementary DNA (cDNA). In some embodiments, assembling each VL domain sequence comprises the use of overlap extension PCR (OE-PCR). In some embodiments, assembling each VH domain sequence comprises the use of overlap extension PCR (OE-PCR). In some embodiments, the single fixed heavy chain sequence is a germline sequence selected from the group consisting of IGHJ4, IGHV1-46, IGHV1-69, IGHV3-15, and IGHV3-23. In some embodiments, applying at least one selective pressure comprises applying a heat stress, selection with protein A, selection with protein L, or a combination thereof. In some embodiments, the heat stress is a temperature of at least 65° C. In some embodiments, applying a heat stress to the plurality of phages eliminates unstable and aggregation prone phages from the subset of phages.

Further provided herein is an antibody library comprising a plurality of antibodies, wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) a CDR sequence is selected from the group consisting of: a VH-CDR1 sequence, a VH-CDR2 sequence, a VH-CDR3 sequence, a VL-CDR1 sequence, a VL-CDR2 sequence, and a VL-CDR3 sequence, wherein the CDR sequence is the same for each antibody of the plurality of antibodies; and (d) a unique combination of remaining CDR sequences are selected from the group consisting of: a VH-CDR1 sequence, a VH-CDR2 sequence, a VH-CDR3 sequence, a VL-CDR1 sequence, a VL-CDR2 sequence, and a VL-CDR3 sequence. In some embodiments, the CDR sequence of (c) is a VH-CDR3 sequence. In some embodiments, the remaining CDR sequences of (d) are a VH-CDR1 sequence, a VH-CDR2 sequence, a VL-CDR1 sequence, a VL-CDR2 sequence, and a VL-CDR3 sequence. In some embodiments, the CDR sequence of (c) is the same as a CDR sequence derived from an initial antibody clone. In some embodiments, each one of the remaining CDR sequences of (d) is present in the antibody library at a high degree of diversity. In some embodiments, the high degree of diversity comprises at least 1×10³ different CDR sequences. In some embodiments, at least one of the VH-CDR1 sequence, the VH-CDR2 sequence, the VH-CDR3 sequence, the VL-CDR1 sequence, the VL-CDR2 sequence, and the VL-CDR3 sequence comprises at least 80% sequence homology to a naturally occurring CDR sequence. In some embodiments, the naturally occurring CDR sequence is derived from a human population. In some embodiments, the remaining CDR sequences of (d) are present in non-naturally occurring combinations for each antibody of the plurality of antibodies. In some embodiments, at least one antibody of the plurality of antibodies has at least one of the following: a higher melting temperature (Tm) as compared to an initial antibody clone, a higher affinity for a target epitope as compared to an initial antibody clone, or a higher cross-reactivity for a target epitope across two or more species as compared to an initial antibody clone. In some embodiments, at least one antibody of the plurality of antibodies has a melting temperature (Tm) that is between about 50° C. and about 90° C. In some embodiments, at least one antibody of the plurality of antibodies binds to a target epitope with a K_{d} of 100 nM or less.

Further provided herein is a method for generating an antibody library, the method comprising: (a) selecting a CDR sequence, wherein the CDR sequence is selected from the group consisting of: a VH-CDR1 sequence, a VH-CDR2 sequence, a VH-CDR3 sequence, a VL-CDR1 sequence, a VL-CDR2 sequence, and a VL-CDR3 sequence; (b) replacing a CDR sequence for each antibody of a first antibody library with the CDR sequence selected in (a), thereby generating a second antibody library comprising a plurality of antibodies, wherein each antibody of the plurality of antibodies comprises: (i) the CDR sequence selected in (a); and (ii) a unique combination of remaining CDR sequences not selected in (a), wherein the remaining CDR sequences are selected from the group consisting of: a VH-CDR1 sequence, a VH-CDR sequence, a VH-CDR3 sequence, a VL-CDR1 sequence, a VL-CDR2 sequence, and a VL-CDR3 sequence. In some embodiments, the first antibody library comprises a plurality of antibodies, wherein each antibody of the plurality of antibodies comprises a unique combination of a VH-CDR1 sequence, a VH-CDR2 sequence, a VH-CDR3 sequence, a VL-CDR1 sequence, a VL-CDR2 sequence, and a VL-CDR3 sequence. In some embodiments, the CDR sequence selected in (a) is a VH-CDR3 sequence. In some embodiments, the remaining CDR sequences of (ii) are a VH-CDR1 sequence, a VH-CDR2 sequence, a VL-CDR1 sequence, a VL-CDR2 sequence, and a VL-CDR3 sequence. In some embodiments, each one of the remaining CDR sequences of (ii) is present in the antibody library at a high degree of diversity. In some embodiments, the high degree of diversity comprises at least 1×10³ different CDR sequences. In some embodiments, at least one of a VH-CDR1 sequence, a VH-CDR2 sequence, a VH-CDR3 sequence, a VL-CDR1 sequence, a VL-CDR2 sequence, and a VL-CDR3 sequence of the antibody library comprises at least 80% sequence homology to a naturally occurring CDR sequence. In some embodiments, the naturally occurring CDR sequence is derived from a human population. In some embodiments, the remaining CDR sequences of (ii) are present in non-naturally occurring combinations for each antibody of the plurality of antibodies. In some embodiments, the CDR sequence of (a) is derived from an initial antibody clone. In some embodiments, at least one antibody of the antibody library has at least one of the following: a higher melting temperature (Tm) as compared to an initial antibody clone, a higher affinity for a target epitope as compared to an initial antibody clone, or higher cross-reactivity for a target epitope across two or more species as compared to an initial antibody clone. In some embodiments, at least one antibody of the antibody library has a melting temperature (Tm) that is between about 50° C. and about 90° C. In some embodiments, at least one antibody of the antibody library binds to a target epitope with a K_{d} of 100 nM or less. In some embodiments, the first antibody library is an antibody library according to any antibody library of the preceding. In some embodiments, the second antibody library is an antibody library according to any one of the preceding. In some embodiments, the method further comprises (c) screening the second antibody library for an antibody with a desired property.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the amount of VH-CDR3 and VL-CDR3 diversity obtained from a single individual for naïve B-cells compared to the amount of VH-CDR1, VH-CDR2, VH-CDR3, VL-CDR1, VL-CDR2, and VL-CDR3 diversity obtained from memory B-cells.

FIG. 2 illustrates the length of time to develop antibody libraries using different technologies, including SuperHuman+Carterra and SuperHuman Zero−Day.

FIG. 3 illustrates the percentage of clones showing affinity (nM) to PD1.

FIG. 4 illustrates reactivity against human and cynomolgus monkey cell surface PD1 against five anti-PD1 clones. In the control, the PPE control/parental cells and the PPE control/transfected cells are the left most peak, while the positive control antibody/transfected cells are the right most peak in each plot. In the selected clones plot, the PPE control/transfected cells are the left most peak, while the PPE positive/transfected cells are the right most peak in each plot.

FIG. 5 illustrates cross-reactivity between human, mouse, and cynomolgus monkey of two anti-PD1 clones.

FIG. 6 illustrates a screen for ligand blockade.

FIG. 7 illustrates a beta galactosidase (bGal) ELISA and Sanger screening of 2 plates comprising 61 positives and 49 unique clones.

FIG. 8 illustrates the diversity in sequences of antibody clones.

FIG. 9 shows antibody fusion variants.

FIG. 10 illustrates variants in the VH-CDR1 (CDR-H1) and VH-CDR2 (CDR-H2) of anti-bGal #27.

FIG. 11 depicts factors involved in framework selection strategy.

FIGS. 12A-12B illustrate framework usage of mAbs from phase I clinical trials. FIG. 12A illustrates heavy chain frameworks used in over 400 mAbs from Phase I clinical trials. FIG. 12B illustrates light chain frameworks used in over 400 mAbs from Phase I clinical trials, showing a majority of phase I mAbs are kappa-derived

FIG. 13 depicts allele frequencies of 12 frameworks in 14 human populations.

FIG. 14 depicts allele frequencies of 27 frameworks in 14 human subpopulations.

FIG. 15 illustrates the affinity maturation landscape of human antibody frameworks.

FIG. 16 illustrates amino acid sequences of 3 framework regions: VH-FR1 (FW1), VH-FR2 (FW2), and VH-FR3 (FW3), and 2 CDRs: VH-CDR1 (CDR-H1) and VH-CDR2 (CDR-H2).

FIG. 17 illustrates the combination of the design and selection to produce an antibody library with functionally diverse VH and VK sequences.

FIG. 18 illustrates heavy chain redundancy in various library preparation processes.

FIG. 19 shows sequence overlap between clones.

FIG. 20 depicts the somatic hypermutation (SHM) from more than 100 individuals.

FIG. 21 describes frameworks (also referred to herein as scaffolds) used and diversity of an antibody library.

FIG. 22 describes the features of an antibody library.

FIG. 23 illustrates observed vs. expected paired mutation frequencies in the VH-CDR1 (CDR-H1) and VH-CDR2 (CDR-H2) regions.

FIG. 24 illustrates IGHV3-23 positional biases.

FIG. 25 shows frequencies of twin pairs.

FIG. 26 depicts IGHV1-3 allele frequency variation in 14 human populations.

FIG. 27 illustrates that less than 10,000 clones dominate the total amount of clones from a peripheral sample of human blood.

FIG. 28 illustrates a phagemid vector expressing an antibody described herein fused to a gIII coat protein.

FIG. 29 depicts a non-limiting example of a method of generating an antibody library as described herein.

FIG. 30 depicts a non-limiting example of an antibody library as described herein.

FIG. 31A and FIG. 31B depict a non-limiting example of a method of screening an antibody library of the disclosure for antibodies having improvements in various characteristics.

FIG. 32 depicts a non-limiting example workflow of a method of generating an antibody library as described herein, along with selection of one or more desired antibodies therefrom.

FIG. 33 depicts a non-limiting example of a method of generating an antibody library as described herein.

FIG. 34 depicts a non-limiting example of a method of screening an antibody library of the disclosure for antibodies having improvements in various characteristics.

FIG. 35 depicts a non-limiting example of a method of screening an antibody library of the disclosure for antibodies having improvements in various characteristics.

### DETAILED DESCRIPTION OF THE INVENTION

A desirable feature of an antibody library can be a high degree of functional diversity. Functional diversity can ensure that not only are a large number of antibodies available for testing purposes, but that this diversity is functionally relevant, thus increasing the utility of these libraries for therapeutic, diagnostic, and research use. Increasing the diversity of a library can be achieved by using naturally occurring complementarity determining regions (CDRs) in combinations that do not naturally occur, such as mixing CDRs from memory and naïve cells, which increases the number of possible CDR combinations. Increasing the functionality of this diversity can further be achieved by selecting for functionality (e.g., the ability to bind to proteins) during the antibody library preparation process.

Disclosed herein, in certain cases, are antibodies having unique properties, antibody libraries comprising a high degree of functional diversity, and methods of making said antibodies and said antibody libraries.

### Antibodies

Antibodies can be synthesized by a B-cell in vivo. Antibody isotypes synthesized by B-cells include, but are not limited to, IgA, IgD, IgE, IgG, and IgM. A B-cell which has not yet encountered an antigen can be termed a naïve B-cell, while B-cells which have encountered and been activated by an antigen can be termed a memory B-cell. Naïve B-cells can express IgM, IgD, or a combination thereof. Memory B-cells can express IgE, IgA, IgG, IgM, or a combination thereof. The IgA can be IgA1 or IgA2. The IgG can be IgG1, IgG2, IgG3, or IgG4. The memory B-cell can be a class switched memory B-cell or a non-switched or marginal zone memory B-cell. The non-switched or marginal zone memory B-cell can express IgM.

A complementarity determining region (“CDR”) is a part of an immunoglobulin (antibody) variable region that can be responsible for the antigen binding specificity of the antibody. A heavy chain (HC) variable region can comprise three CDR regions, abbreviated VH-CDR1, VH-CDR2, and VH-CDR3 and found in this order on the heavy chain from the N terminus to the C terminus; and a light chain (LC) variable region can comprise three CDR regions, abbreviated VL-CDR1, VL-CDR2, and VL-CDR3 and found in this order on the light chain from the N terminus to the C terminus. Further, the light chain can be a kappa chain (VK) or a lambda chain (Vλ). Surrounding and interspersed between the CDRs are framework regions which can contribute to the structure and can display less variability than the CDR regions.

A heavy chain variable region can comprise four framework regions, abbreviated VH-FR1, VH-FR2, VH-FR3, and VH-FR4. The heavy chain can comprise, from N to C terminus: VH-FR1 VH-CDR1 VH-FR2 VH-CDR2 VH-FR3 VH-CDR3 VH-FR4. A light chain variable region can comprise four framework regions, abbreviated VL-FR1, VL-FR2, VL-FR3, and VL-FR4. The light chain can comprise, from N to C terminus: VL-FR1 VL-CDR1 VL-FR2 VL-CDR2 VL-FR3 VL-CDR3 VL-FR4. In some cases, “CDR sequence” as used herein, refers to a CDR sequence selected from the group consisting of: VH-CDR1, VH-CDR2, VH-CDR3, VL-CDR1, VL-CDR2, VL-CDR3, and any combination thereof.

### Radically Diverse Antibody Libraries

Antibody libraries described herein can comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (a) at least one of the VH-CDR3 sequence and the VL-CDR3 sequence is derived from a naïve B-cell; (b) if only one of the VH-CDR3 and VL-CDR3 is derived from the naïve B-cell, then the VH-CDR3 or VL-CDR3 not derived from the naïve B-cell is derived from a memory cell; and (c) the VH-CDR1 sequence, VH-CDR2 sequence, VL-CDR1 sequence, and VL-CDR2 sequence are derived from a memory cell. The antibody library can also be referred to herein as a SuperHuman Library.

In some cases, the plurality of antibodies in the antibody library has high functional diversity. An antibody library with high functional diversity can comprise a plurality of antibodies wherein at least 80%, 85%, 90%, 95%, or 99% of the plurality of antibodies are functional. Functional antibodies can be antibodies with the ability to bind to a protein. The ability of an antibody to bind to a protein can be determined by screening the antibody against protein A or protein L. An antibody library with high functional diversity can comprise a plurality of antibodies wherein at least 80% of the plurality of antibodies are functional. An antibody library with high functional diversity can comprise a plurality of antibodies wherein at least 85% of the plurality of antibodies are functional. An antibody library with high functional diversity can comprise a plurality of antibodies wherein at least 90% of the plurality of antibodies are functional. An antibody library with high functional diversity can comprise a plurality of antibodies wherein at least 95% of the plurality of antibodies are functional. An antibody library with high functional diversity can comprise a plurality of antibodies wherein at least 99% of the plurality of antibodies are functional.

The antibody library can comprise at least 1.0×10⁵, 2.0×10⁵, 3.0×10⁵, 4.0×10⁵, 5.0×10⁵ 6.0×10⁵, 7.0×10⁵, 8.0×10⁵, 9.0×10⁵, 1.0×10¹⁰, 2.0×10¹⁰, 3.0×10¹⁰, 4.0×10¹⁰, 5.0×10¹⁰, 6.0×10¹⁰, 7.0×10¹⁰, 8.0×10¹⁰, or 9.0×10¹⁰ antibodies. The antibody library can comprise at least 1.0×10⁵ antibodies. The antibody library can comprise at least 7.0×10¹⁰ antibodies. The antibody library can comprise at least 7.1×10¹⁰, 7.2×10¹⁰, 7.3×10¹⁰, 7.4×10¹⁰, 7.5×10¹⁰, 7.6×10¹⁰, 7.7×10¹⁰, 7.8×10¹⁰, or 7.9×10¹⁰ antibodies. The antibody library can comprise at least 7.6×10¹⁰ antibodies.

In some instances, the antibody library comprises at least 1.0×10⁵ antibodies of which at least 80% of the plurality of antibodies are functional. In some instances, the antibody library comprises at least 7.0×10¹⁰ antibodies of which at least 80% of the plurality of antibodies are functional. In some instances, the antibody library comprises at least 7.6×10¹⁰ antibodies of which at least 80% of the plurality of antibodies are functional.

In some instances, the antibody library comprises at least 1.0×10⁵ antibodies of which at least 85% of the plurality of antibodies are functional. In some instances, the antibody library comprises at least 7.0×10¹⁰ antibodies of which at least 85% of the plurality of antibodies are functional. In some instances, the antibody library comprises at least 7.6×10¹⁰ antibodies of which at least 85% of the plurality of antibodies are functional.

In some instances, the antibody library comprises at least 1.0×10⁵ antibodies of which at least 90% of the plurality of antibodies are functional. In some instances, the antibody library comprises at least 7.0×10¹⁰ antibodies of which at least 90% of the plurality of antibodies are functional. In some instances, the antibody library comprises at least 7.6×10¹⁰ antibodies of which at least 90% of the plurality of antibodies are functional.

In some instances, the antibody library comprises at least 1.0×10⁵ antibodies of which at least 95% of the plurality of antibodies are functional. In some instances, the antibody library comprises at least 7.0×10¹⁰ antibodies of which at least 95% of the plurality of antibodies are functional. In some instances, the antibody library comprises at least 7.6×10¹⁰ antibodies of which at least 95% of the plurality of antibodies are functional.

In some instances, the antibody library comprises at least 1.0×10⁵ antibodies of which at least 99% of the plurality of antibodies are functional. In some instances, the antibody library comprises at least 7.0×10¹⁰ antibodies of which at least 99% of the plurality of antibodies are functional. In some instances, the antibody library comprises at least 7.6×10¹⁰ antibodies of which at least 99% of the plurality of antibodies are functional.

The antibodies of the library can comprise non-naturally occurring combinations of naturally occurring CDRs, such as combinations of CDRs derived from naturally occurring memory B-cells and naïve B-cells, but whose joint appearance on the same antibody would not be naturally occurring. For example, a non-naturally occurring combination of naturally occurring CDRs can comprise at least one CDR derived from a naïve cell while the remaining CDRs can be derived from a memory cell. For example, a non-naturally occurring combination of naturally occurring CDRs can comprise at least one CDR derived from cells of predominantly naïve B-cell origin while the remaining CDRs can be derived from cells of predominantly memory B-cell origin. Naturally occurring CDRs can refer to CDRs naturally occurring in a human population.

The non-naturally occurring combination of naturally occurring CDRs can comprise at least one CDR derived from a naïve cell, while the remaining CDRs are derived from a memory cell. In some cases, at least VL-CDR1 is derived from a naïve cell. In some cases, at least VL-CDR2 is derived from a naïve cell. In some cases, at least VL-CDR3 is derived from a naïve cell. In some cases, at least VH-CDR1 is derived from a naïve cell. In some cases, at least VH-CDR2 is derived from a naïve cell. In some cases, at least VH-CDR3 is derived from a naïve cell.

The non-naturally occurring combination of naturally occurring CDRs can comprise two, three, four, or five CDRs derived from a naïve cell, while the remaining CDRs can be derived from a memory cell. For example, two CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, three CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, four CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, five CDRs from CDRs in the group consisting of: VL-CDR1, VL-CDR2, VL-CDR3, VH-CDR1, VH-CDR2, and VH-CDR3 can be derived from a naïve cell while the remaining CDR can be derived from a memory cell.

In another non-limiting example of a non-naturally occurring combination, VL-CDR3 can be derived from a naïve cell, while VH-CDR1, VH-CDR2, VH-CDR3, VL-CDR1, and VL-CDR2 can be derived from a memory cell. In another non-limiting example of a non-naturally occurring combination, VH-CDR3 can be derived from a naïve cell, while VH-CDR1, VH-CDR2, VL-CDR1, VL-CDR2, and VL-CDR3 can be derived from a memory cell. In another non-limiting example of a non-naturally occurring combination, VH-CDR3 and VL-CDR3 can be derived from a naïve cell, while VH-CDR1, VH-CDR2, VL-CDR1, and VL-CDR2 can be derived from a memory cell.

Amino acid residues in an antibody sequence, a variable heavy chain sequence of the antibody, or a variable light chain sequence of the antibody can be referred to in terms of their Kabat position. “Kabat position,” as used herein, can refer to the numbering system described in Kabat et al., 1991, in Sequences of Proteins of Immunological Interest, 5th edition, US Department of Health and Human Services, NIH, USA. In some cases, an antibody described herein comprises a variation at Kabat position H93, Kabat position H94, or a combination thereof. In some cases, at least one antibody in an antibody library comprise a variation at Kabat position H93, Kabat position H94, or a combination thereof. In some cases, at least 5%, 10%, 15%, 20%, 25%, 30%, 35%, 40%, 45%, or 50% of the antibodies in an antibody library comprise a variation at Kabat position H93, Kabat position H94, or a combination thereof. The variation can be a mutation, insertion, or deletion.

“Derived,” when used in reference to a sequence, can refer to any CDR sequence with sequence homology to a naturally occurring CDR sequence of at least 80%, at least 85%, at least 90%, at least 95%, at least 99%, or 100%. “Derived” can refer to any CDR sequence obtained from sequencing information obtained from a pool of cells of predominantly naïve B-cell origin or a pool of cells of predominantly memory B-cell origin. For instance, a sequence is “derived” from a cell if (1) a sequence was observed in the cell and (2) the same sequence (or a sequence having at least 80%, at least 85%, at least 90%, at least 95%, at least 99% or at least 100% sequence homology to the sequence) is chemically synthesized based on the observed sequence.

A VH-CDR1 sequence derived from a naïve B-cell can comprise at least 80%, 85%, 90%, 95%, or 99% sequence homology to a naturally occurring VH-CDR1 sequence from a naïve B-cell. A VH-CDR1 sequence derived from a naïve B-cell can be a synthetic VH-CDR1 sequence. A VH-CDR1 sequence derived from a naïve B-cell can comprise 100% sequence homology to a naturally occurring VH-CDR1 sequence from a naïve B-cell. A VH-CDR2 sequence derived from a naïve B-cell can comprise at least 80%, 85%, 90%, 95%, or 99% sequence homology to a naturally occurring VH-CDR2 sequence from a naïve B-cell. A VH-CDR2 sequence derived from a naïve B-cell can be a synthetic VH-CDR2 sequence. A VH-CDR2 sequence derived from a naïve B-cell can comprise 100% sequence homology to a naturally occurring VH-CDR2 sequence from a naïve B-cell. A VH-CDR3 sequence derived from a naïve B-cell can comprise at least 80%, 85%, 90%, 95%, or 99% sequence homology to a naturally occurring VH-CDR3 sequence from a naïve B-cell. A VH-CDR3 sequence derived from a naïve B-cell can be a synthetic VH-CDR3 sequence. A VH-CDR3 sequence derived from a naïve B-cell can comprise 100% sequence homology to a naturally occurring VH-CDR3 sequence from a naïve B-cell.

A VL-CDR1 sequence derived from a naïve B-cell can comprise at least 80%, 85%, 90%, 95%, or 99% sequence homology to a naturally occurring VL-CDR1 sequence from a naïve B-cell. A VL-CDR1 sequence derived from a naïve B-cell can be a synthetic VL-CDR1 sequence. A VL-CDR1 sequence derived from a naïve B-cell can comprise 100% sequence homology to a naturally occurring VL-CDR1 sequence from a naïve B-cell. A VL-CDR2 sequence derived from a naïve B-cell can comprise at least 80%, 85%, 90%, 95%, or 99% sequence homology to a naturally occurring VL-CDR2 sequence from a naïve B-cell. A VL-CDR2 sequence derived from a naïve B-cell can be a synthetic VL-CDR2 sequence. A VL-CDR2 sequence derived from a naïve B-cell can comprise 100% sequence homology to a naturally occurring VL-CDR2 sequence from a naïve B-cell. A VL-CDR3 sequence derived from a naïve B-cell can comprise at least 80%, 85%, 90%, 95%, or 99% sequence homology to a naturally occurring VL-CDR3 sequence from a naïve B-cell. A VL-CDR3 sequence derived from a naïve B-cell can be a synthetic VL-CDR3 sequence. A VL-CDR3 sequence derived from a naïve B-cell can comprise 100% sequence homology to a naturally occurring VL-CDR3 sequence from a naïve B-cell.

The VH-CDR1 sequence, VH-CDR2 sequence, VH-CDR3 sequence, VL-CDR1 sequence, VL-CDR2 sequence, VL-CDR3 sequence, or any combination thereof can be derived from sequence information obtained from a pool of cells of predominantly naïve B-cell origin. The VH-CDR3 sequence, the VL-CDR3 sequence, or the combination thereof can be derived from sequence information obtained from a pool of cells of predominantly naïve B-cell origin. The pool of naïve B-cells can be obtained from a plurality of individuals. The pool of naïve B-cells can comprise less than 0.1%, 1%, 5%, 10%, 20%, or 30% cells not of naïve B-cell origin.

A VH-CDR1 sequence derived from a memory B-cell can comprise at least 80%, 85%, 90%, 95%, or 99% sequence homology to a naturally occurring VH-CDR1 sequence from a memory B-cell. A VH-CDR1 sequence derived from a memory B-cell can be a synthetic VH-CDR1 sequence. A VH-CDR1 sequence derived from a memory B-cell can comprise 100% sequence homology to a naturally occurring VH-CDR1 sequence from a memory B-cell. A VH-CDR2 sequence derived from a memory B-cell can comprise at least 80%, 85%, 90%, 95%, or 99% sequence homology to a naturally occurring VH-CDR2 sequence from a memory B-cell. A VH-CDR2 sequence derived from a memory B-cell can be a synthetic VH-CDR2 sequence. A VH-CDR2 sequence derived from a memory B-cell can comprise 100% sequence homology to a naturally occurring VH-CDR2 sequence from a memory B-cell. A VH-CDR3 sequence derived from a memory B-cell can comprise at least 80%, 85%, 90%, 95%, or 99% sequence homology to a naturally occurring VH-CDR3 sequence from a memory B-cell. A VH-CDR3 sequence derived from a memory B-cell can be a synthetic VH-CDR3 sequence. A VH-CDR3 sequence derived from a memory B-cell can comprise 100% sequence homology to a naturally occurring VH-CDR3 sequence from a memory B-cell.

A VL-CDR1 sequence derived from a memory B-cell can comprise at least 80%, 85%, 90%, 95%, or 99% sequence homology to a naturally occurring VL-CDR1 sequence from a memory B-cell. A VH-CDR1 sequence derived from a memory B-cell can be a synthetic VH-CDR1 sequence. A VL-CDR1 sequence derived from a memory B-cell can comprise 100% sequence homology to a naturally occurring VL-CDR1 sequence from a memory B-cell. A VL-CDR2 sequence derived from a memory B-cell can comprise at least 80%, 85%, 90%, 95%, or 99% sequence homology to a naturally occurring VL-CDR2 sequence from a memory B-cell. A VL-CDR2 sequence derived from a memory B-cell can comprise 100% sequence homology to a naturally occurring VL-CDR2 sequence from a memory B-cell. A VL-CDR3 sequence derived from a memory B-cell can comprise at least 80%, 85%, 90%, 95%, or 99% sequence homology to a naturally occurring VL-CDR3 sequence from a memory B-cell. A VL-CDR3 sequence derived from a memory B-cell can comprise 100% sequence homology to a naturally occurring VL-CDR3 sequence from a memory B-cell.

The VH-CDR1 sequence, VH-CDR2 sequence, VH-CDR3 sequence, VL-CDR1 sequence, VL-CDR2 sequence, VL-CDR3 sequence, or any combination thereof can be derived from sequence information obtained from a pool of cells of predominantly memory B-cell origin. The VH-CDR3 sequence, the VL-CDR3 sequence, or the combination thereof can be derived from sequence information obtained from a pool of cells of predominantly memory B-cell origin. The pool of memory B-cells can be obtained from a plurality of individuals. The pool of memory B-cells can comprise less than 0.1%, 1%, 5%, 10%, 20%, or 30% cells not of memory B-cell origin. The memory B-cells can be CD27+ B-cells. The pool of memory B-cells can comprise less than 0.1%, 1%, 5%, 10%, 20%, or 30% cells not of CD27+ B-cell origin.

The percent sequence homology can be calculated by determining the number of positions at which the identical nucleic acid base occurs in two sequences to yield the number of matched positions, dividing the number of matched positions by the total number of positions, which can include additions or deletions, and multiplying the result by 100 to yield the percent sequence homology. Percent sequence homology, also referred to as percent sequence identity, can be determined by aligning each sequence in any suitable sequence alignment program, such as Clustal Omega, Multiple Sequence Comparison by Log-Expectation (MUSCLE), Multiple Alignment using Fast Fourier Transform (MAFFT), MegAlign, and Basic Local Alignment Search Tool (BLAST).

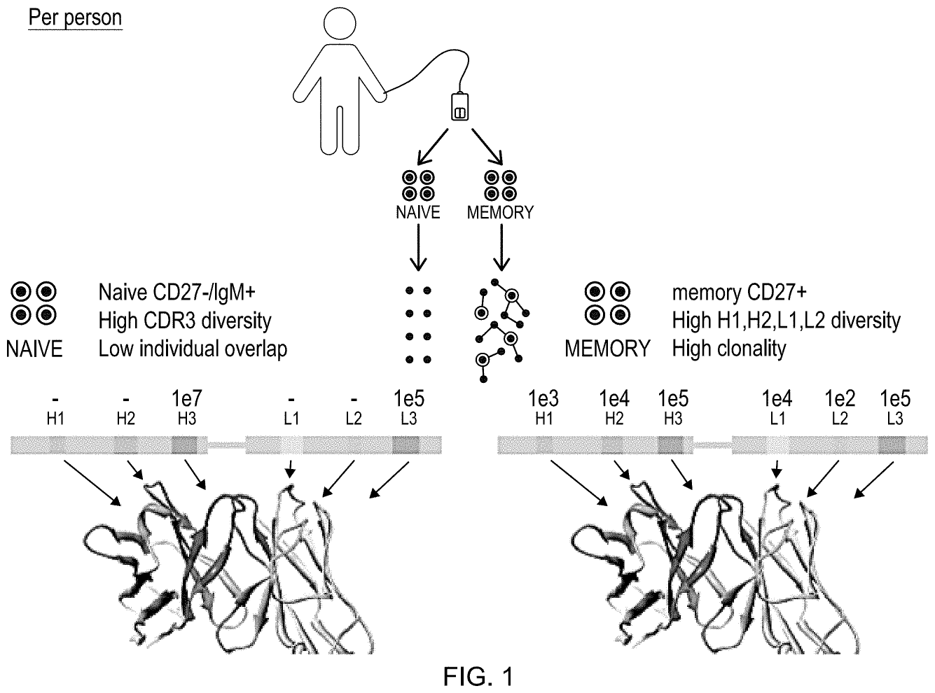

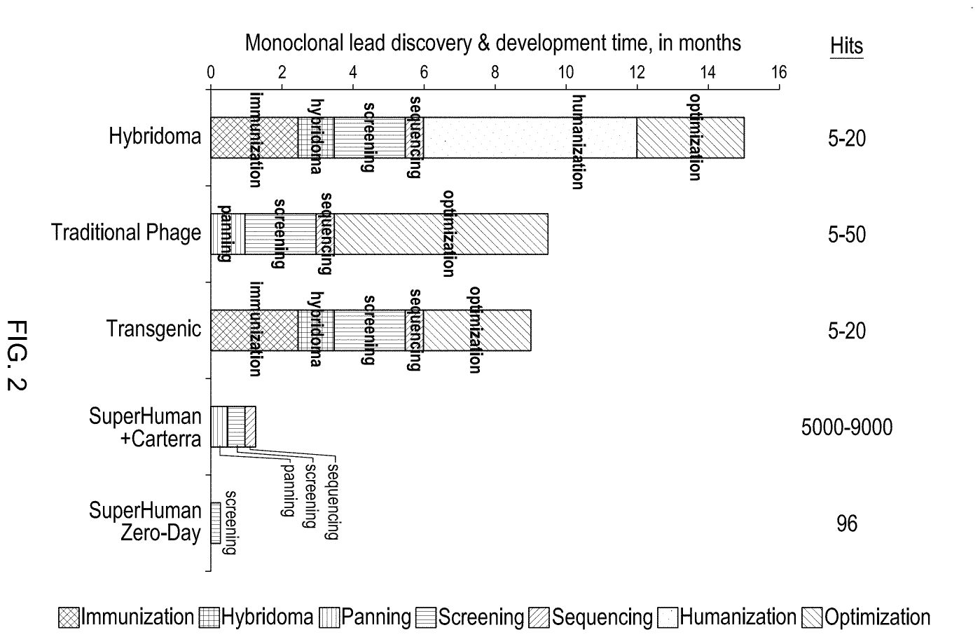

The naïve cell can be a naïve B-cell. The naïve B-cell can be a human naïve B-cell. The memory cell can be a memory B-cell. The memory B-cell can be a human memory B-cell. In some instances, a naïve B-cell shows increased diversity of VH-CDR3 and VL-CDR3 sequences compared to VH-CDR3 and VL-CDR3 sequences from a memory B-cell (FIG. 1). The naïve cells and the memory cells can be obtained from a biological sample, such as blood, from an individual or a plurality of individuals. The naïve cells and the memory cells can be physically isolated from this sample using a marker specific to the naïve cells or the memory cells.

A marker can be used to identify, separate, or sort B-cells, naïve B-cells, and memory B-cells from a biological sample. Examples of markers used to identify, separate, or sort a B-cell include, but are not limited to, CD19+. Examples of markers used to identify, separate, or sort a naïve B-cell include, but are not limited to, CD19+, CD27−, IgD+, IgM+, and combinations thereof. Examples of markers used to identify, separate, or sort a memory B-cell include, but are not limited to, CD19+, CD27+, and combinations thereof. In some embodiments, CD27+ is used to sort memory B-cells. Examples of markers used to identify, separate, or sort a class switched memory B-cell include, but are not limited to CD19+, CD27+, CD27+, IgD−, IgM−, and combinations thereof. Examples of markers used to identify, separate, or sort a nonswitched or marginal zone memory B-cell include, but are not limited to, CD19+, CD27+, IgD+, IgM+, and combinations thereof. In some cases, a memory B-cell can be identified, separated, or sorted with the following markers: CD19+, CD27+, IgD−, IgM+, and combinations thereof. The naïve cell from which the VH-CDR3 is derived can be a CD27−/IgM+ B-cell. The memory cell from which the VH-CDR1, VH-CDR2, VL-CDR1, VL-CDR2, and VL-CDR3 are derived can be a CD27+/IgG+ B-cell.

The CDR sequences of the antibody can be CDR sequences found in naïve B-cells and memory B-cells found in an individual or a plurality of individuals. The individual can be a mammal. The mammal can be a human, a non-human primate, mouse, rat, pig, goat, rabbit, horse, cow, cat, or dog. In some cases, the CDR sequences are CDR sequences obtained from a publically available source. Examples of publically available sources of CDR sequences include SAbDab (http://opig.stats.ox.ac.uk/webapps/sabdab-sabpred/Welcome.php) and PylgClassify (http://dunbrack2.fccc.edu/PyIgClassify/).

A germline antibody sequence can comprise germline framework and germline CDR sequences. Each CDR in an antibody found in the antibody library can comprise at least 1, 2, 3, or 4 mutations compared to a corresponding germline CDR region. Each CDR in an antibody in the antibody library can comprise no more than 4 mutations compared to a corresponding framework CDR region.

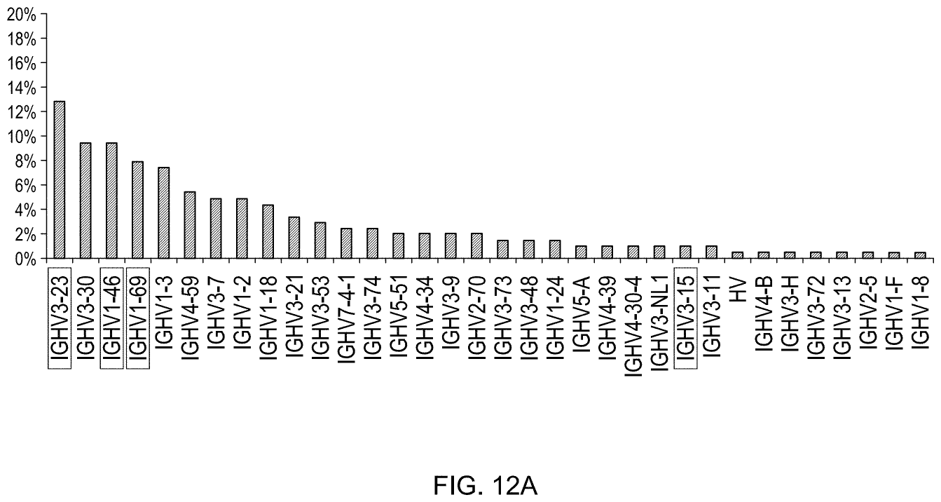

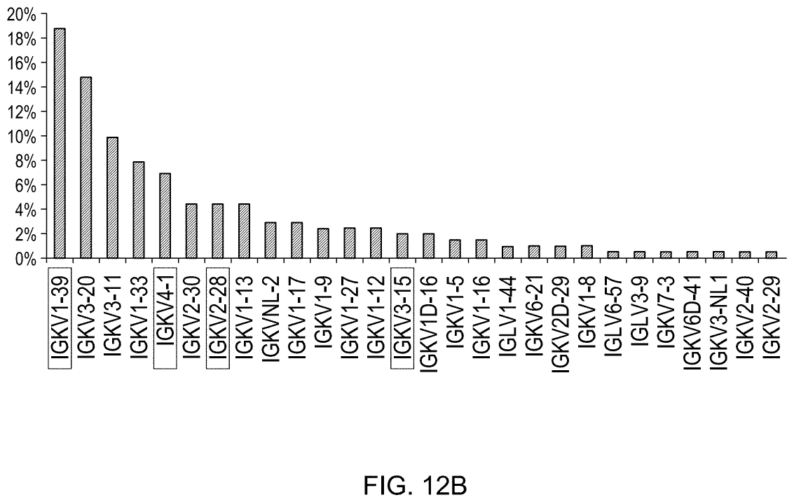

The framework of the antibody can be a naturally occurring framework. The naturally occurring framework can be a framework found in a mammal. The mammal can be a primate, mouse, rat, pig, goat, rabbit, horse, cow, cat, or dog. The primate can be a human. The framework can comprise at least one variant compared to a naturally occurring framework. The variant can be a mutation, an insertion, or a deletion. The variant can be a variant found in the nucleic acid sequence encoding the antibody or a variant found in the amino acid sequence of the antibody. Any suitable framework sequence can be used, such as those previously used in phase I clinical trials (FIGS. 12A, 12B). As used herein, the framework of an antibody can refer to the framework regions of the variable heavy chain (VH-FR1, VH-FR2, VH-FR3, and VH-FR4), the framework regions of the variable heavy chain (VL-FR1, VL-FR2, VL-FR3, and VL-FR4), or a combination thereof. The framework regions of an antibody in the antibody library can be identical to germline framework regions.

The framework can be a therapeutically optimal framework. The therapeutically optimal framework can comprise at least one, at least two, at least three, at least four, at least five, at least six, or all of the following properties selected from the group consisting of: a) previously demonstrated safety in human monoclonal antibodies, b) thermostable; c) not prone to aggregation; d) comprises a single dominant allele at the amino acid level across all human populations; e) comprises different canonical topologies of the CDRs; f) expresses well in bacteria; and g) displays well on a phage. A framework with previously demonstrated safety in human monoclonal antibodies can be a framework of an antibody that has been used in at least a phase I clinical trial. A framework that is thermostable can be a framework that is thermostable at at least 20° C., 30° C., 40° C., 50° C., 60° C., 70° C., 80° C., 90° C., 100° C., or over 100° C. A framework that is thermostable can be a framework that can withstand a temperature increase of at least 3° C. per minute, 4° C. per minute, or 5° C. per minute. A framework that expresses well in bacteria can be a framework that produces a biologically active antibody in the bacteria. The bacteria can be E. coll. The bacteria can be an engineered bacteria. The bacteria can be a bacteria optimized for antibody expression. A framework that displays well on a phage can be a framework that produces a biologically active antibody when displayed on the surface of the phage.

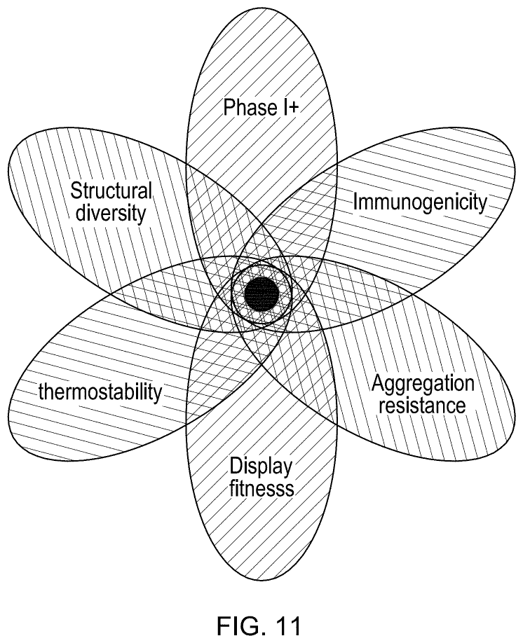

An example of a strategy used to choose a framework is described in FIG. 11, wherein an ideal framework of an antibody can be one that shows structural diversity, has been used successfully in a Phase I clinical trial in humans, has low immunogenicity, shows aggregation resistance, displays fitness, and is thermostable. In some cases, frameworks of antibodies are avoided if they are inherently autoreactive to blood cells (e.g., IGHV4-34), have an inferior stability profile (e.g., IGHV2-5), have a V-gene not found in at least 50% of individuals (e.g., IGHV4-b), show an aggregation prone V-gene (e.g., IGLV6-57), or a combination thereof.

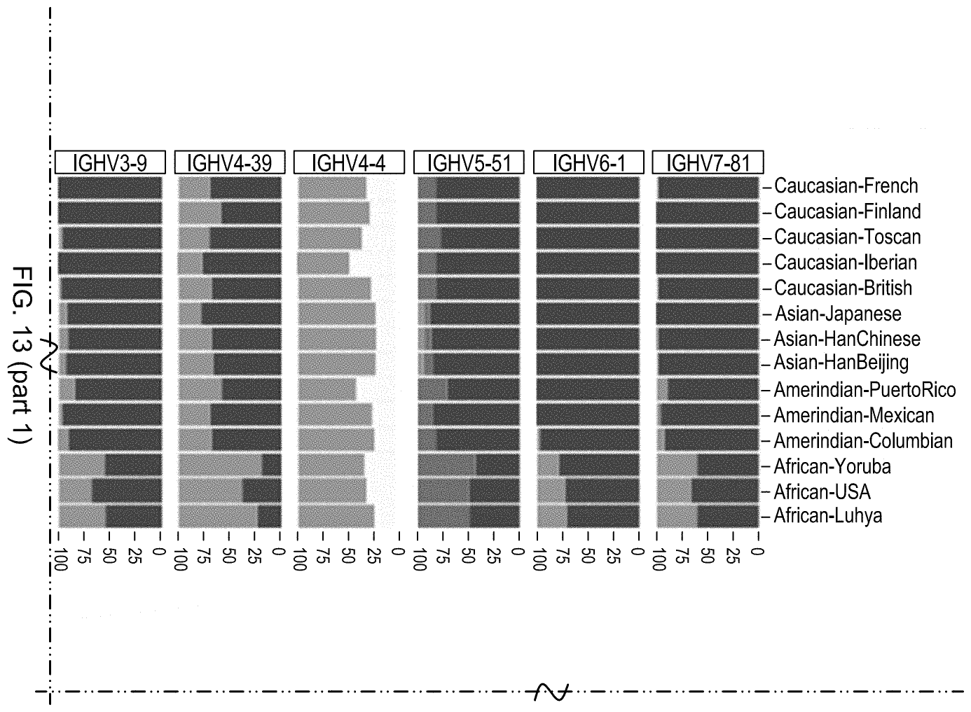

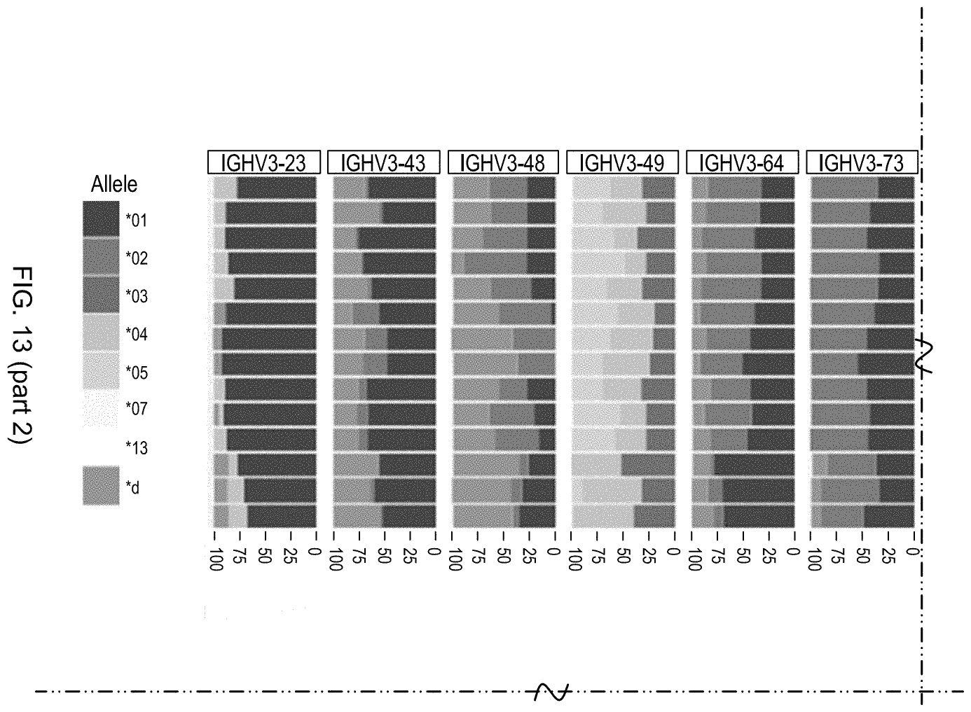

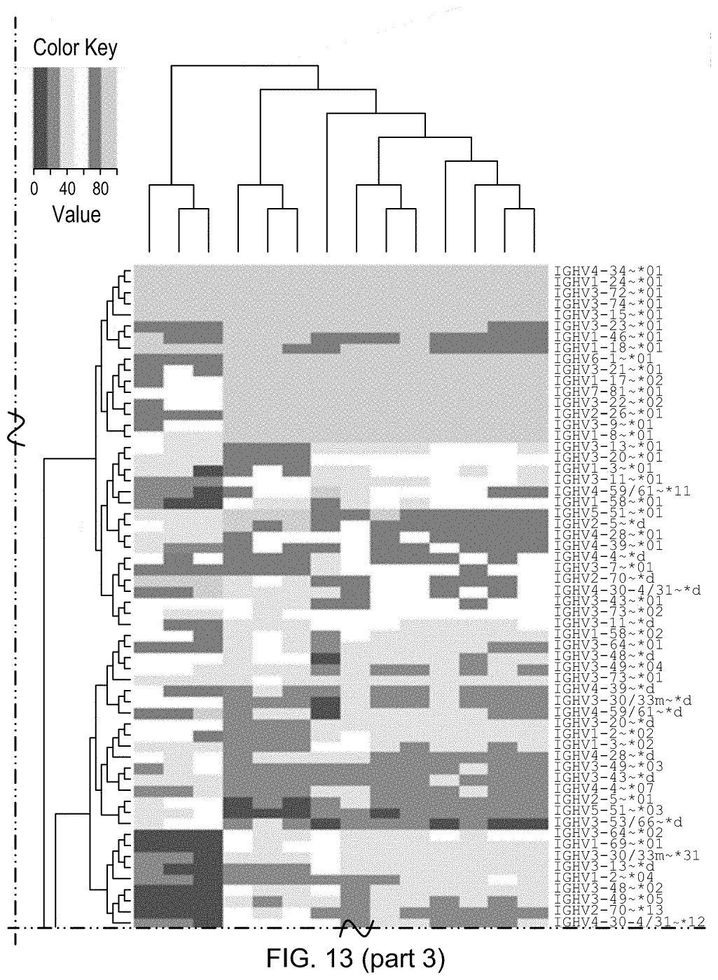

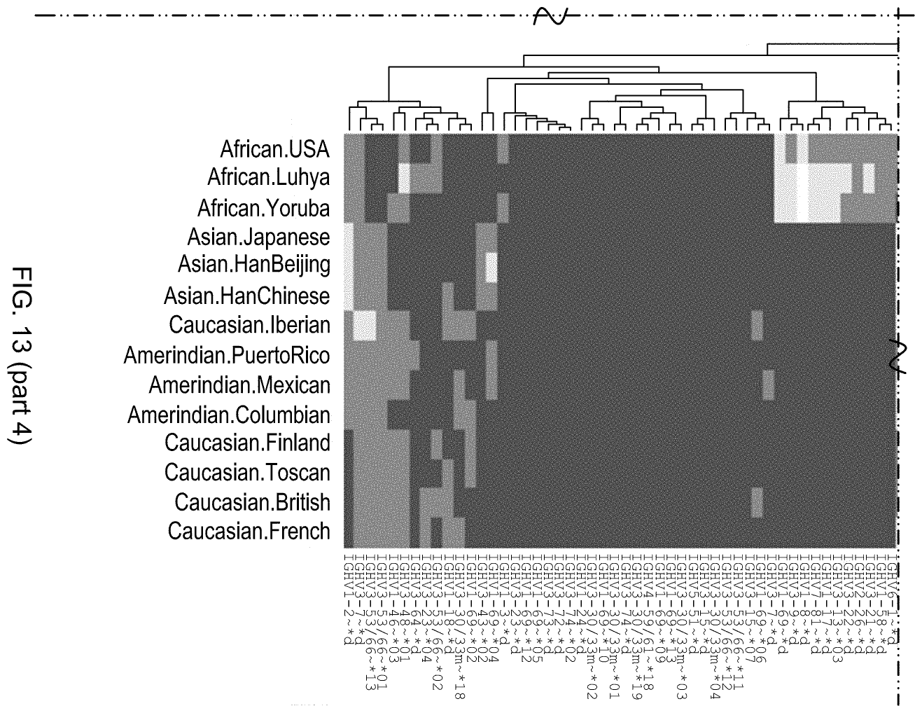

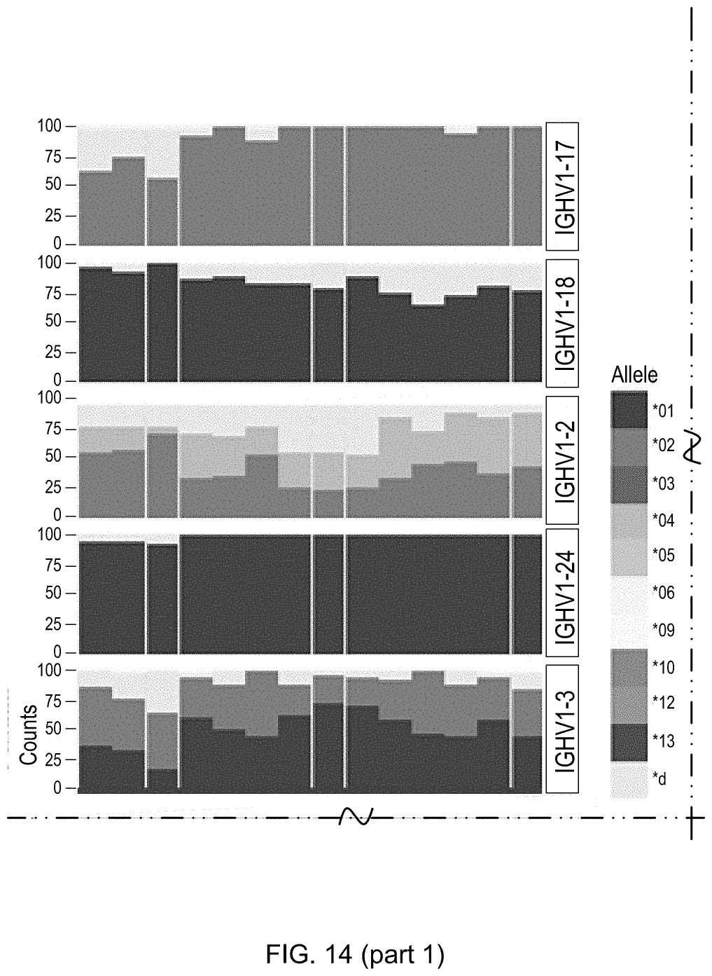

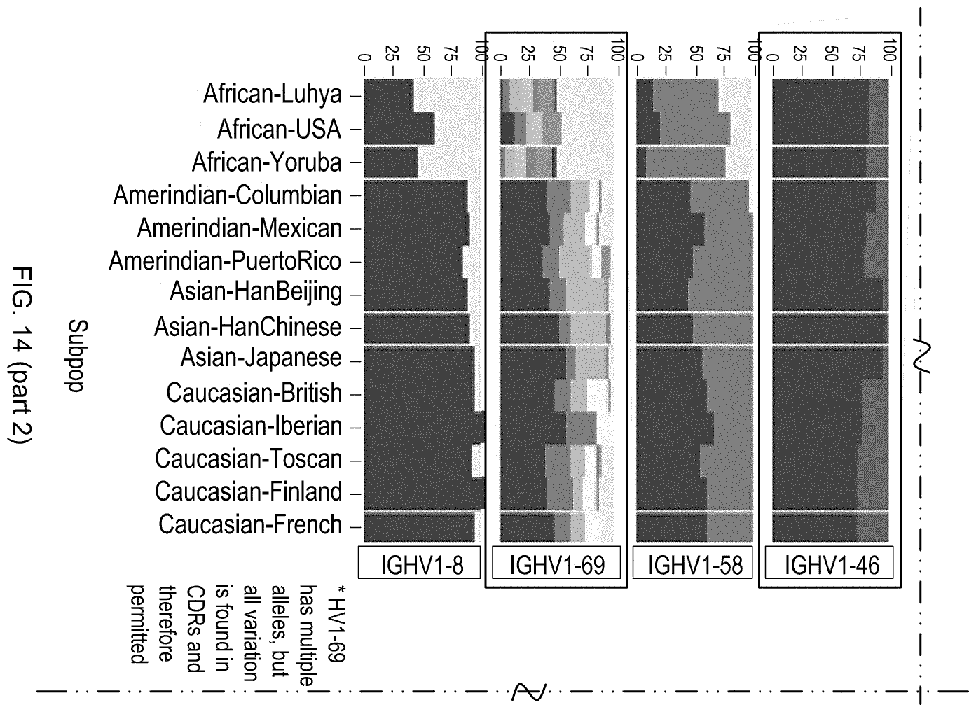

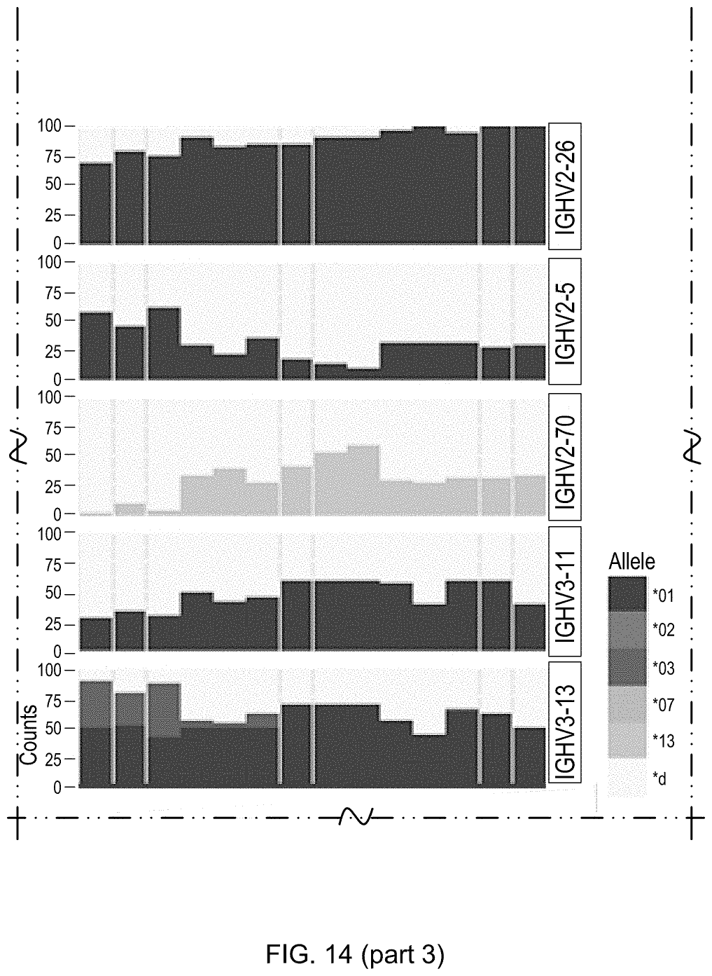

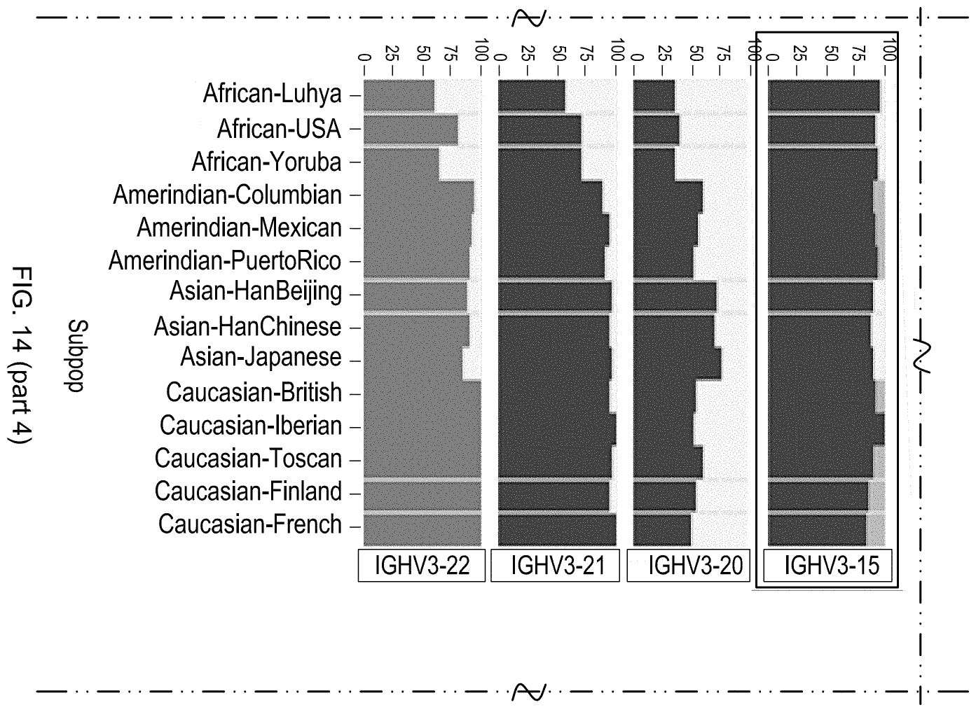

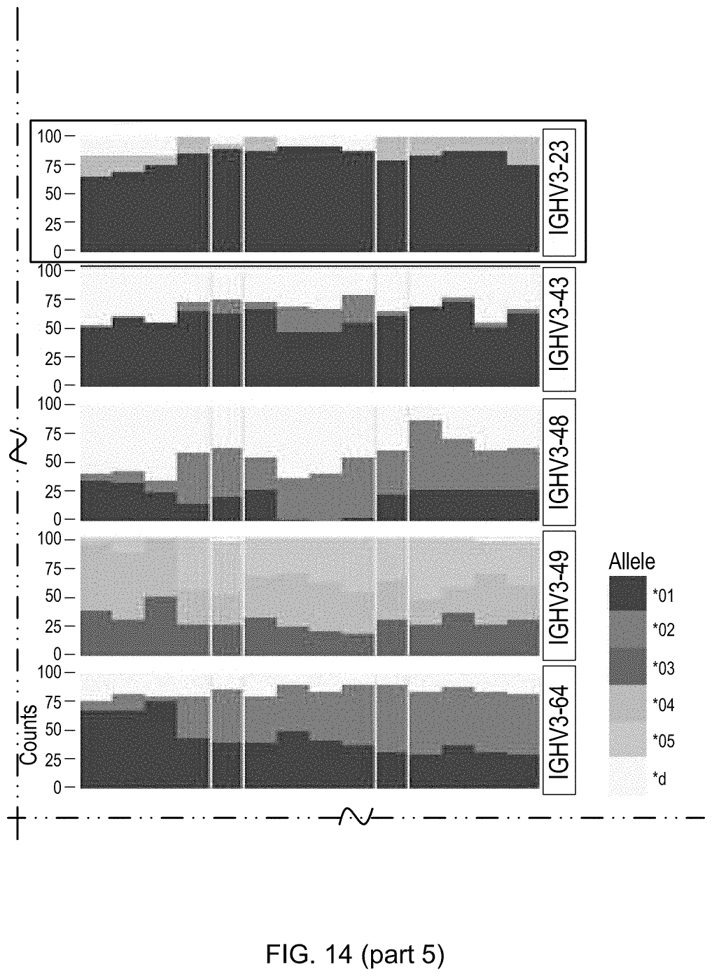

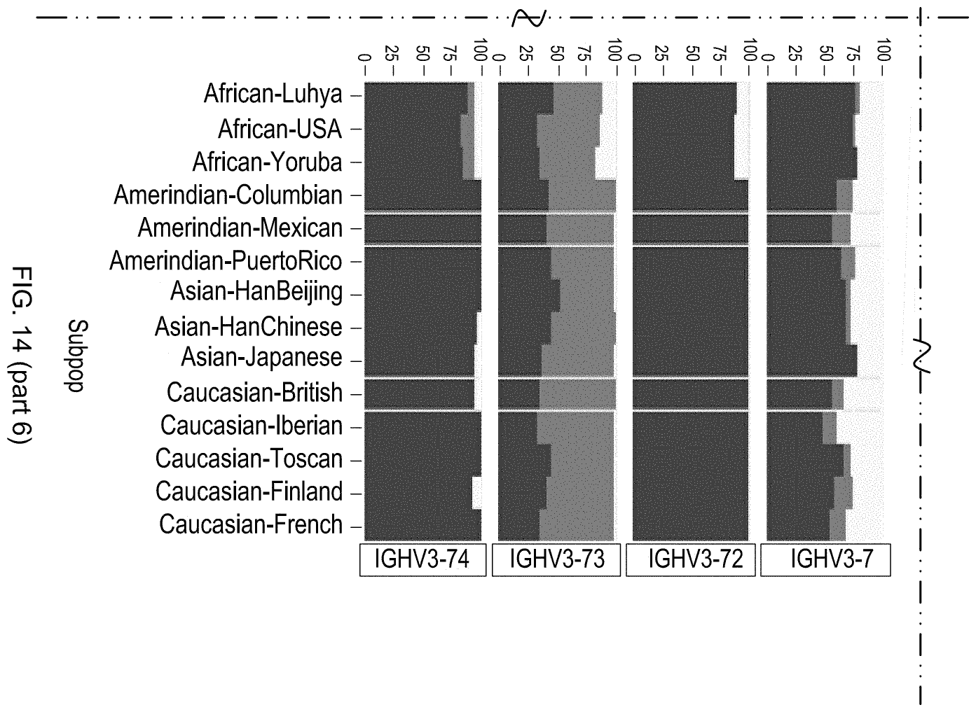

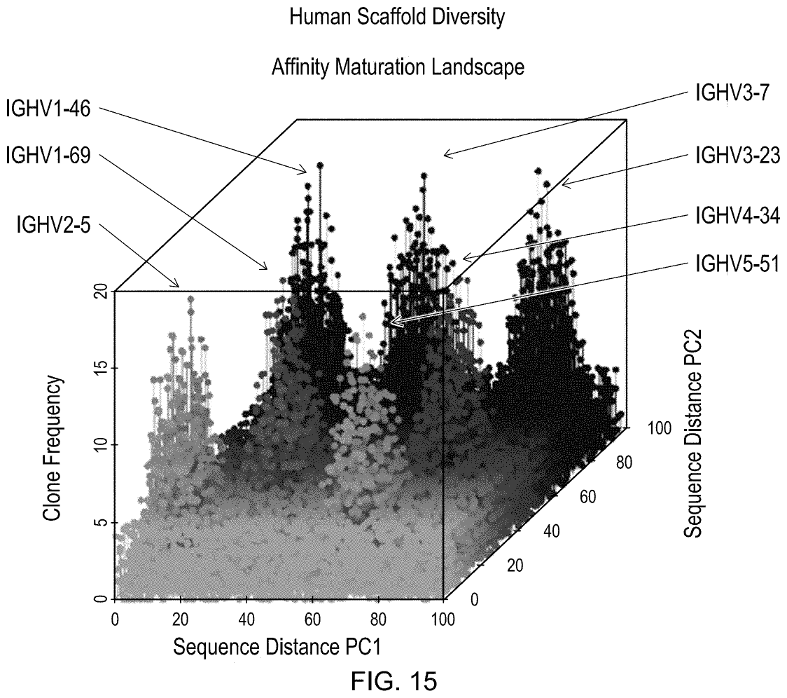

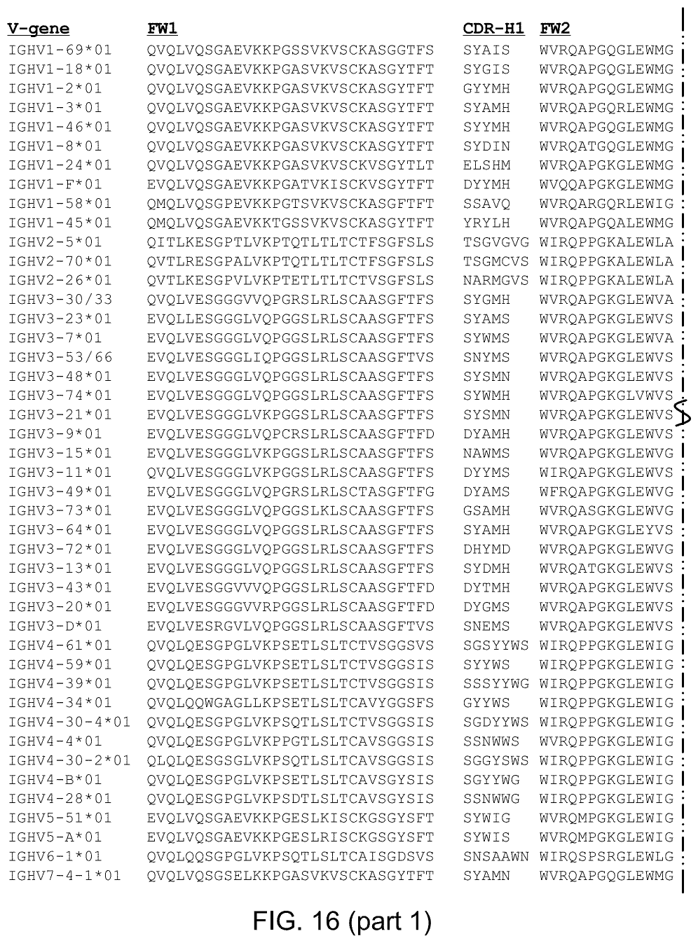

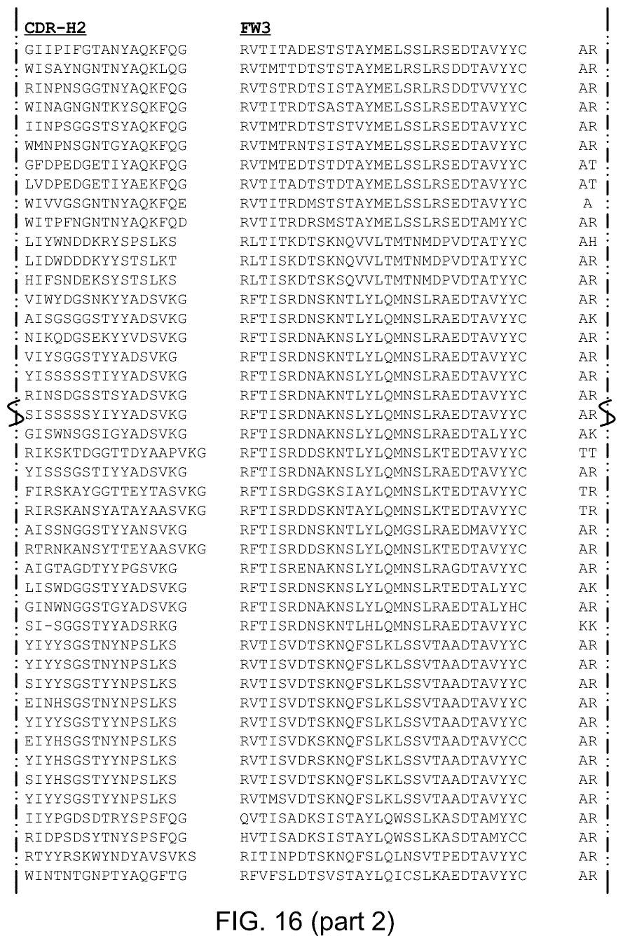

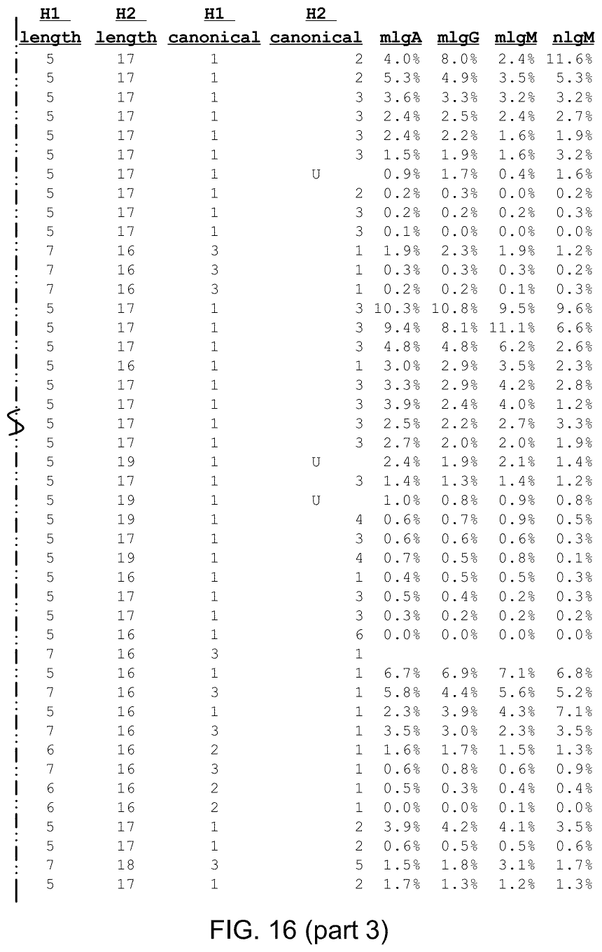

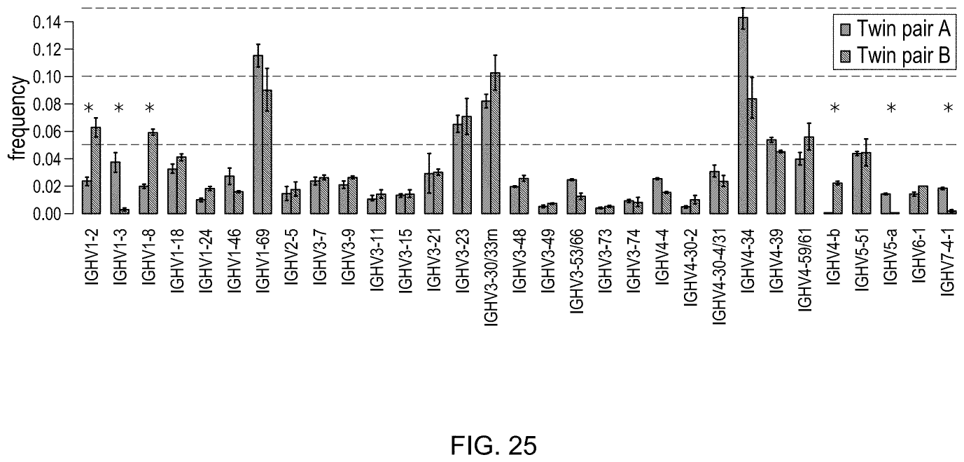

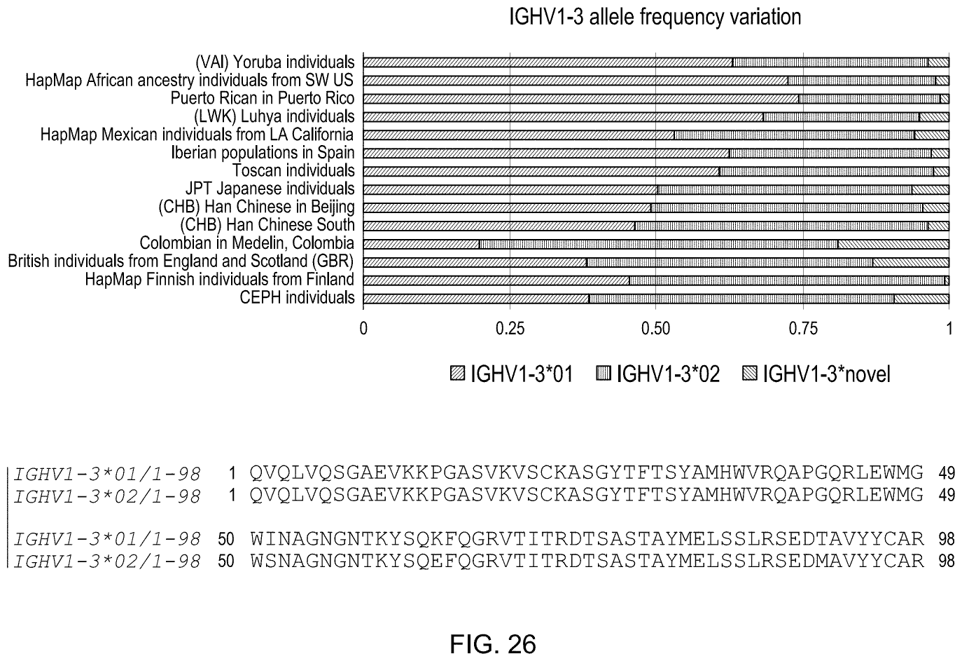

The amino acid sequence of a framework of an antibody herein can comprise more than one dominant allele, wherein there are different dominant alleles in different human populations (FIG. 13 and FIG. 14). For example, the IGHV1-3 framework comprises 3 alleles: IGVH1-3*01, IGVH1-3*02, and IGVH1-3*03, which are found in different frequencies in different human populations (FIG. 26). In some instances, the amino acid sequence of the framework of the antibodies described herein has a single dominant allele in at least two human populations. In some instances, the amino acid sequence of the framework of the antibodies described herein has a single dominant allele in all human populations. A framework with one dominant allele can be a framework in which one allele is found in at least 50%, at least 75%, or at least 90% in at least two human populations. A framework with one dominant allele can be a framework in which one allele is found in at least 50%, at least 75%, or at least 90% in at least twelve human populations. In some cases, the framework regions of the VH domain are framework regions from IGHJ4, IGHV1-46, IGHV1-69, IGHV3-15, or IGHV3-23. In some cases, the framework regions of the VH domain are framework regions from IGHV2-5, IGHV3-7, IGVH4-34, IGHV5-51, IGHV1-24, IGHV2-26, IGHV3-72, IGHV3-74, IGHV3-9, IGHV3-30, IGHV3-33, IGHV3-53, IGHV3-66, IGHV4-30-4, IGHV4-31, IGHV4-59, IGHV4-61, or IGHV5-51. In some cases, the framework regions of the VH domain of the antibodies in the antibody library are framework regions from IGHV1-46, IGHV3-23, or a combination thereof. In some cases, the frameworks regions of the VL domain of the antibodies in the antibody library are framework regions from IGKV1-39, IGKV2-28, IGKV3-15, IGKV4-1, IGKV1-5, IGKV1-12, IGKV1-13, IGKV3-11, IGKV3-20, or a combination thereof. In one example, a subset of antibodies in the antibody library can have framework regions of the VH domain from IGHV1-46 and framework regions of the VL domain from IGKV1-39, while the remaining antibodies in the antibody library have framework regions of the VH domain from IGHV1-46 and framework regions of the VL domain from IGKV2-28.

Disclosed herein, in some cases, are nucleic acid sequences encoding an antibody described herein. The nucleic acid sequence can be a DNA or an RNA sequence. The nucleic acid can be inserted into a vector. The vector can be a phage. The phage can be a phagemid or a bacteriophage. The phagemid can be pMID21. The bacteriophage can be DY3F63, an M13 phage, fd filamentous phage, T4 phage, T7 phage or λ phage. In some cases a phagemid can be introduced into a microbe in combination with a bacteriophage (i.e. a ‘helper’ phage). The microbe can be a filamentous bacteria. The filamentous bacteria can be Escherichia coli.

The antibody libraries described herein comprise a plurality of antibodies. The plurality of antibodies can be at least 1.0×10⁶, 1.0×10⁷, 1.0×10⁸, 1.0×10⁹, 1.0×10¹⁰, 2.0×10¹⁰, 3.0×10¹⁰, 4.0×10¹⁰, 5.0×10¹⁰, 6.0×10¹⁰, 7.0×10¹⁰, 8.0×10¹⁰, 9.0×10¹⁰, or 10.0×10¹⁰ antibodies. The plurality of antibodies can be at least 1.0×10¹¹ antibodies. The plurality of antibodies can be at least 7.6×10¹⁰ antibodies. Such libraries can be unique because of their high diversity. For example, in any of the libraries herein at least 2%, at least 5%, at least 10%, at least 15%, at least 20%, at least 25%, at least 30%, or at least 35% of the plurality of antibodies can be unique. In some instances, a library has more than 7.0×10¹⁰ antibodies of which at least 20% of the plurality of antibodies are unique. The unique antibody can vary by at least one nucleic acid or at least one amino acid residue relative to the other antibodies of the antibody library.

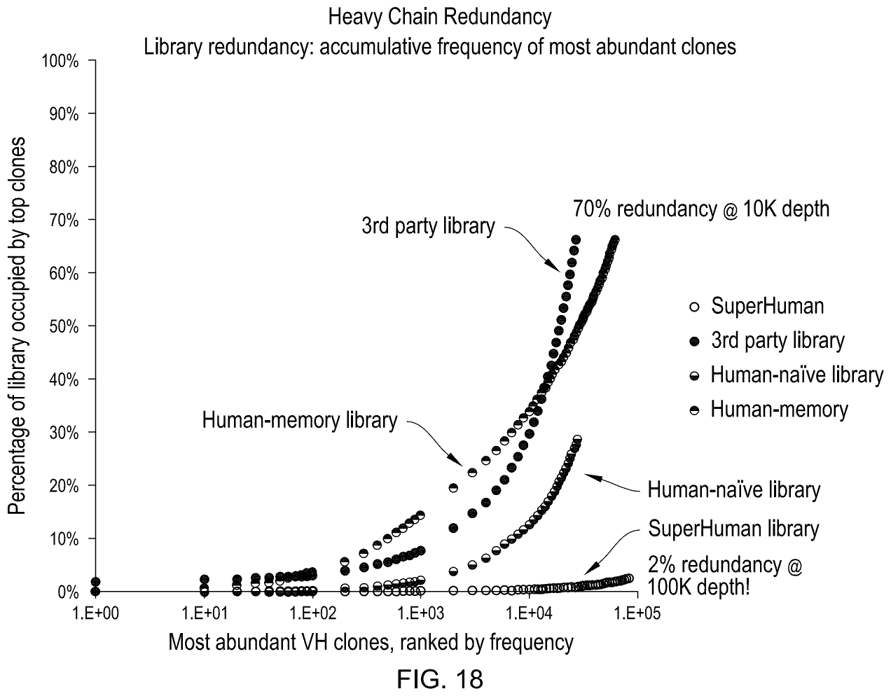

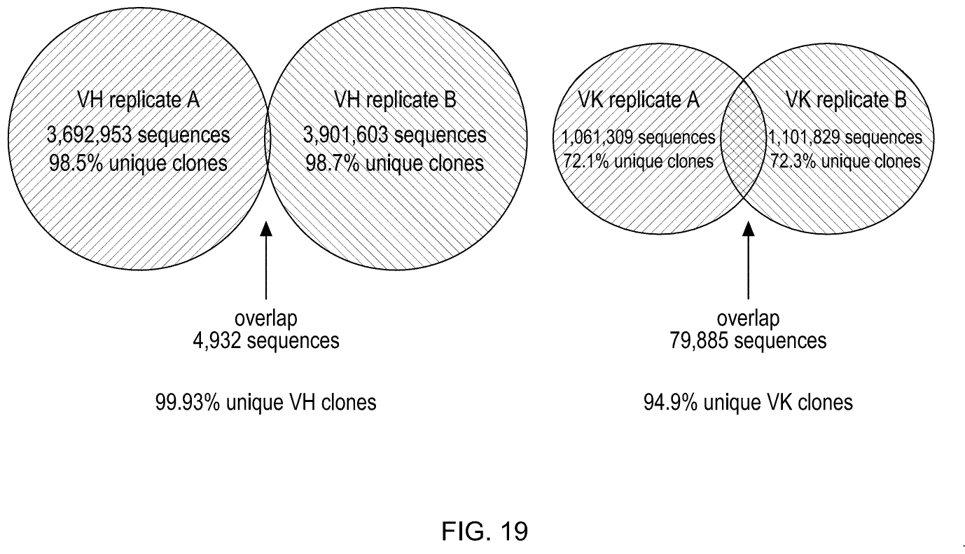

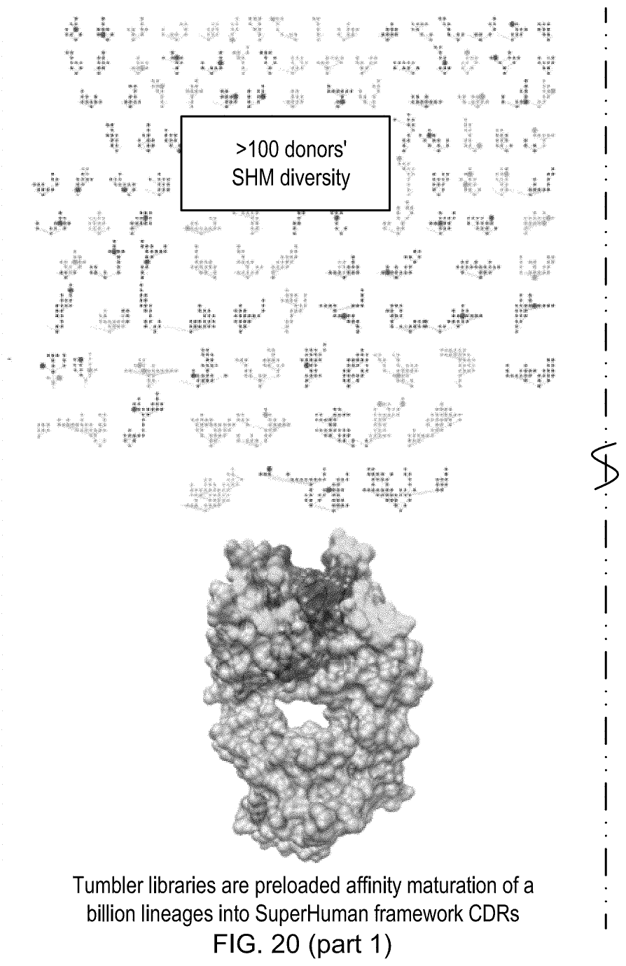

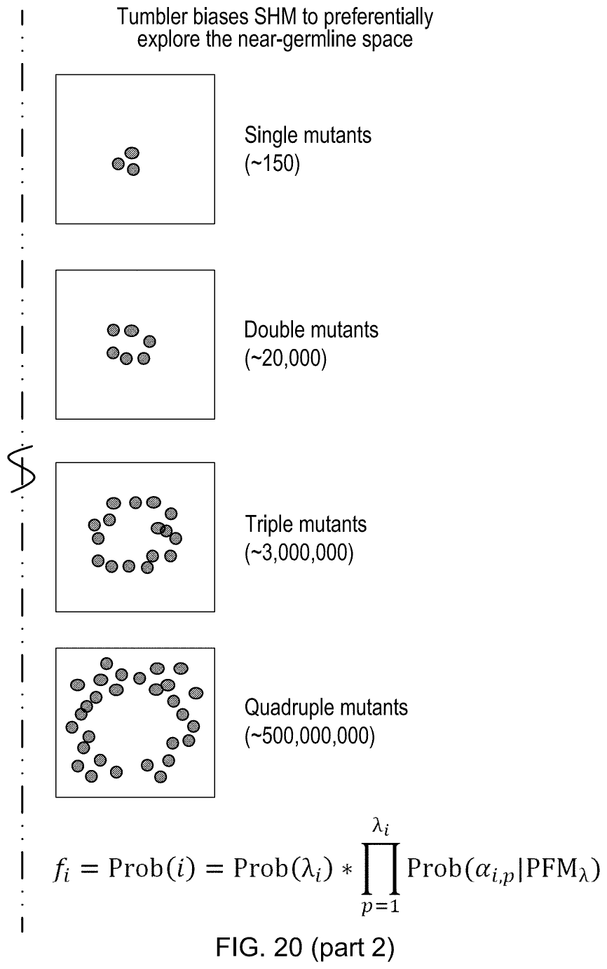

The total amount of antibodies found naturally in the human body (e.g., from naïve cell and memory cell antibodies) as well as antibody libraries produced by other library preparation methods can comprise highly redundant heavy chain sequences (FIG. 18). If one heavy chain sequence of an antibody in a library is redundant with another heavy chain sequence of a different antibody, this can indicate that the heavy chain sequences are identical. Two or more antibodies with redundant heavy chain sequences can comprise different antibody framework regions, different light chain sequences, or a combination thereof. The antibody libraries produced by the methods described herein can show reduced heavy chain redundancy. A reduction in heavy chain redundancy can increase diversity of the antibody library. Redundancy can be measured by percentage of the library occupied by the top clones. The antibody libraries produced herein can have a redundancy of about 2%, about 3%, about 4%, or about 5% (FIG. 18). In some instances, the maximum number of heavy chains of a traditional natural library is limited to 1.0×10⁷ antibodies due to limitations in naturally occurring combinations of CDRs. In some instances, the heavy chains of antibodies in libraries described herein are not limited by naturally occurring combinations of CDRs and can comprise over 1.0×10¹¹ antibodies.

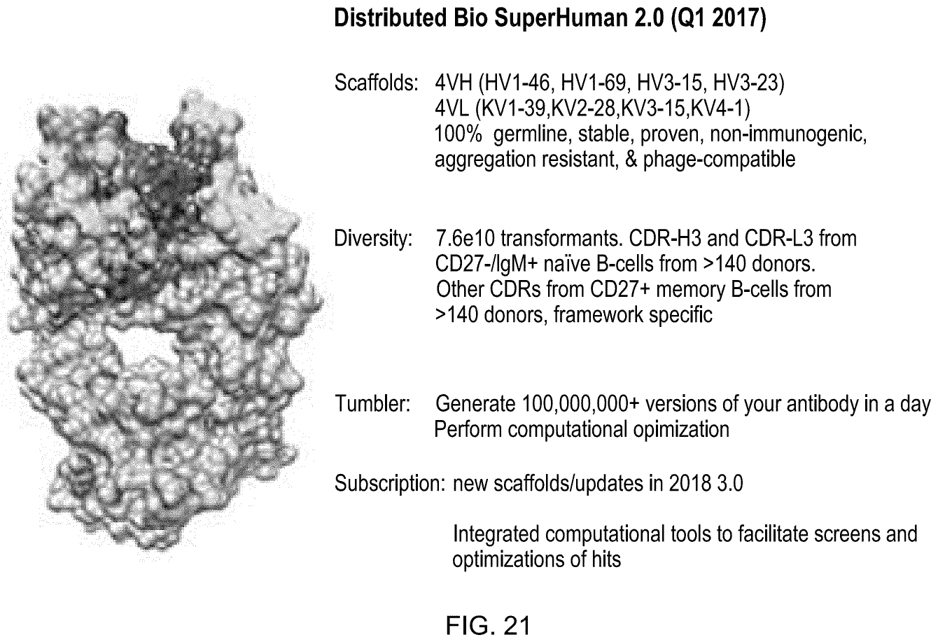

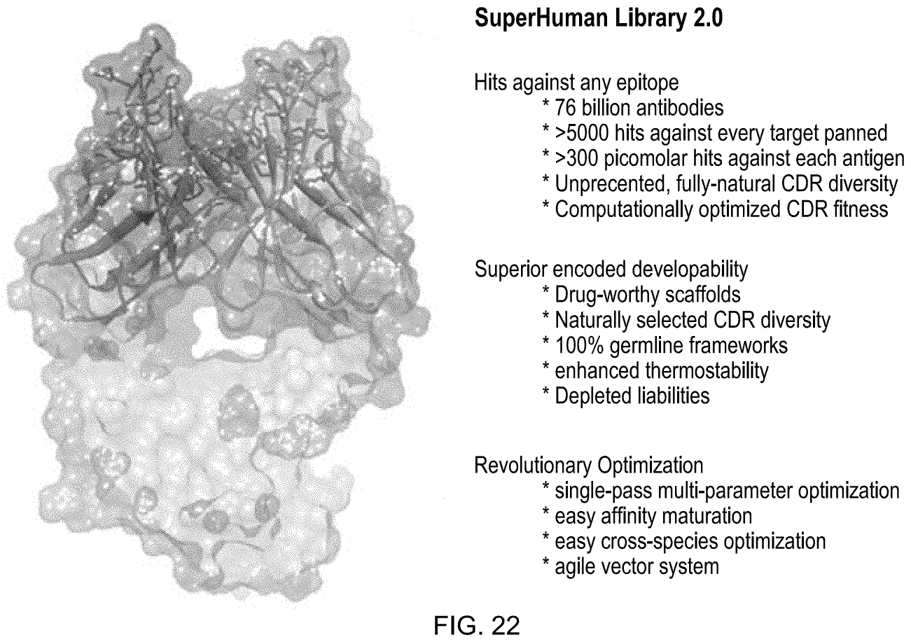

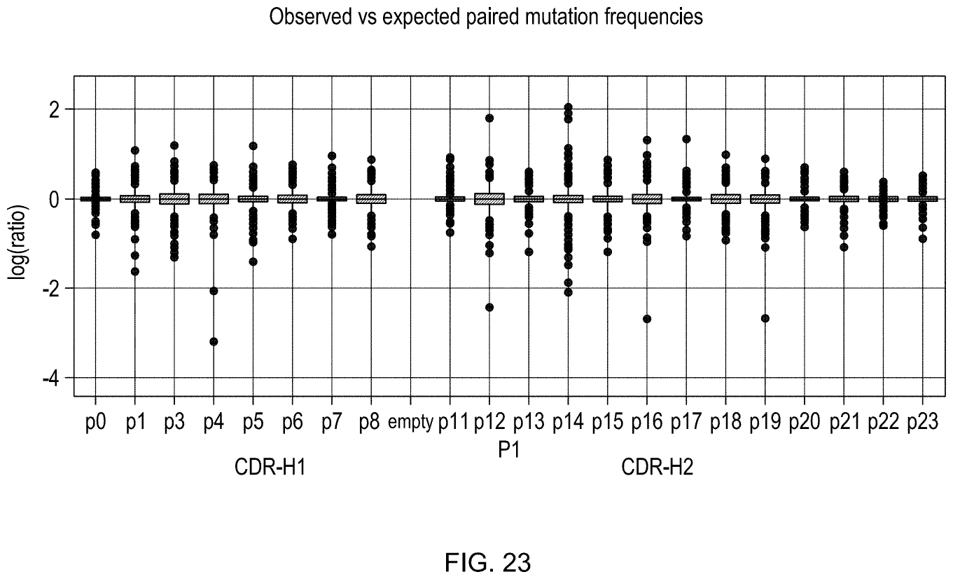

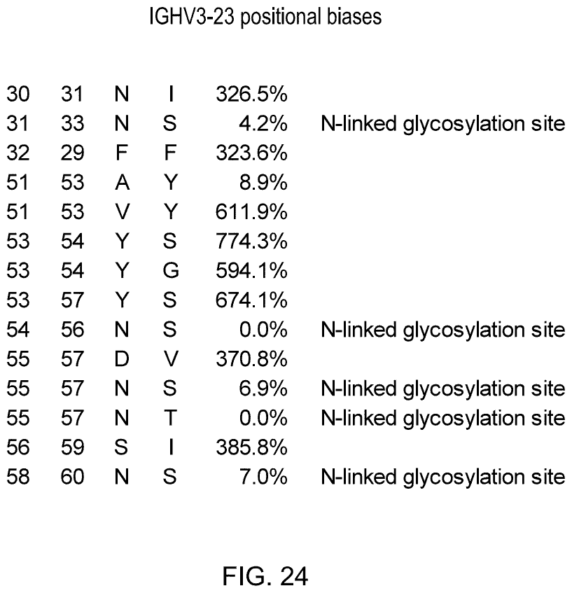

In some instances, the antibody library is an antibody library as described in FIG. 21. In some instances, the antibody library is an antibody library as described in FIG. 22.

### Methods of Generating Diverse Antibody Libraries

Described herein, in certain cases, are methods of preparing an antibody library comprising a plurality of antibodies, wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (a) at least one of the VH-CDR3 sequence and the VL-CDR3 sequence is derived from a naïve B-cell; (b) the VH-CDR3 sequence or the VL-CDR3 sequence not derived from a naïve B-cell is derived from a memory B-cell; and (c) the VH-CDR1 sequence, VH-CDR2 sequence, VL-CDR1 sequence, and VL-CDR2 sequence are derived from a memory cell.

In some cases, methods described herein produce an antibody library having high functional diversity. An antibody library with high functional diversity can comprise a plurality of antibodies wherein at least 80%, 85%, 90%, 95%, or 99% of the plurality of antibodies are functional. Functional antibodies can be antibodies with the ability to bind to a protein. The ability of an antibody to bind to a protein can be determined by screening the antibody against protein A or protein L. An antibody library with high functional diversity can comprise a plurality of antibodies wherein at least 90% of the plurality of antibodies are functional. An antibody library with high functional diversity can comprise a plurality of antibodies wherein at least 95% of the plurality of antibodies are functional. An antibody library with high functional diversity can comprise a plurality of antibodies wherein at least 99% of the plurality of antibodies are functional.

The antibody library can comprise at least 1.0×10⁵, 2.0×10⁵, 3.0×10⁵, 4.0×10⁵, 5.0×10⁵ 6.0×10⁵, 7.0×10⁵, 8.0×10⁵, 9.0×10⁵, 1.0×10¹⁰, 2.0×10¹⁰, 3.0×10¹⁰, 4.0×10¹⁰, 5.0×10¹⁰, 6.0×10¹⁰, 7.0×10¹⁰, 8.0×10¹⁰, or 9.0×10¹⁰ antibodies. The antibody library can comprise at least 1.0×10⁵ antibodies. The antibody library can comprise at least 7.0×10¹⁰ antibodies. The antibody library can comprise at least 7.1×10¹⁰, 7.2×10¹⁰, 7.3×10¹⁰, 7.4×10¹⁰, 7.5×10¹⁰, 7.6×10¹⁰, 7.7×10¹⁰, 7.8×10¹⁰, or 7.9×10¹⁰ antibodies. The antibody library can comprise at least 7.6×10¹⁰ antibodies.

In some instances, the antibody library comprises at least 1.0×10⁵ antibodies of which at least 80% of the plurality of antibodies are functional. In some instances, the antibody library comprises at least 7.0×10¹⁰ antibodies of which at least 80% of the plurality of antibodies are functional. In some instances, the antibody library comprises at least 7.6×10¹⁰ antibodies of which at least 80% of the plurality of antibodies are functional.

In some instances, the antibody library comprises at least 1.0×10⁵ antibodies of which at least 85% of the plurality of antibodies are functional. In some instances, the antibody library comprises at least 7.0×10¹⁰ antibodies of which at least 85% of the plurality of antibodies are functional. In some instances, the antibody library comprises at least 7.6×10¹⁰ antibodies of which at least 85% of the plurality of antibodies are functional.

In some instances, the antibody library comprises at least 1.0×10⁵ antibodies of which at least 90% of the plurality of antibodies are functional. In some instances, the antibody library comprises at least 7.0×10¹⁰ antibodies of which at least 90% of the plurality of antibodies are functional. In some instances, the antibody library comprises at least 7.6×10¹⁰ antibodies of which at least 90% of the plurality of antibodies are functional.

In some instances, the antibody library comprises at least 1.0×10⁵ antibodies of which at least 95% of the plurality of antibodies are functional. In some instances, the antibody library comprises at least 7.0×10¹⁰ antibodies of which at least 95% of the plurality of antibodies are functional. In some instances, the antibody library comprises at least 7.6×10¹⁰ antibodies of which at least 95% of the plurality of antibodies are functional.

In some instances, the antibody library comprises at least 1.0×10⁵ antibodies of which at least 99% of the plurality of antibodies are functional. In some instances, the antibody library comprises at least 7.0×10¹⁰ antibodies of which at least 99% of the plurality of antibodies are functional. In some instances, the antibody library comprises at least 7.6×10¹⁰ antibodies of which at least 99% of the plurality of antibodies are functional.

The method for preparing an antibody library can comprise: (a) obtaining sequence information for a plurality of VH-CDR3 and VL-CDR3 sequences from a pool of naïve B-cells and sequence information for a plurality of VH-CDR1, VH-CDR2, VH-CDR3, VL-CDR1, VL-CDR2, and VL-CDR3 sequences from a pool of memory B-cells; (b) assembling a plurality of variable light (VL) domain sequences, each VL domain sequence comprising a VL-CDR1 sequence derived from the sequence information from memory B-cells determined in step a., a VL-CDR2 sequence derived from the sequence information from memory B-cells determined in step a., and a VL-CDR3 sequence derived from the sequence information from memory B-cells or naïve B-cells determined in step a.; (c) assembling a plurality of first nucleic acid sequences encoding a plurality of first antibodies, each first antibody comprising: (i) a variable light (VL) domain sequence assembled in step b.; and (ii) a single fixed heavy chain sequence; (d) inserting the plurality of first nucleic acid sequences into a plurality of phages; (e) expressing the plurality of first antibodies on the surface of the plurality of phages; (f) applying at least one selective pressure to the plurality of phages to produce a subset of phages comprising a subset of first nucleic acid sequences; (g) assembling a plurality of a variable heavy (VH) domain sequences, each VH domain sequence comprising: a VH-CDR1 sequence derived from the sequence information from memory B-cells determined in step a., a VH-CDR2 sequence derived from the sequence information from memory B-cells determined in step a., and a VH-CDR3 sequence derived from the sequence information from memory B-cells or naïve B-cells determined in step a., wherein at least one of the VH-CDR3 sequence and the VL-CDR3 sequence is derived from the sequence information from naïve B-cells; (h) replacing the single fixed heavy chain sequences from the subset of first nucleic acid sequences with the plurality of VH domain sequences assembled in step g. to produce a plurality of second nucleic acid sequences, each second nucleic acid sequence comprising: (i) a variable light (VL) domain sequence assembled in step b., and (ii) a variable heavy (VH) domain sequence assembled in step g. wherein the plurality of second nucleic acid sequences encodes a plurality of second antibodies; and (i) transforming a plurality of microbes with the plurality of phages to produce a plurality of transformants.

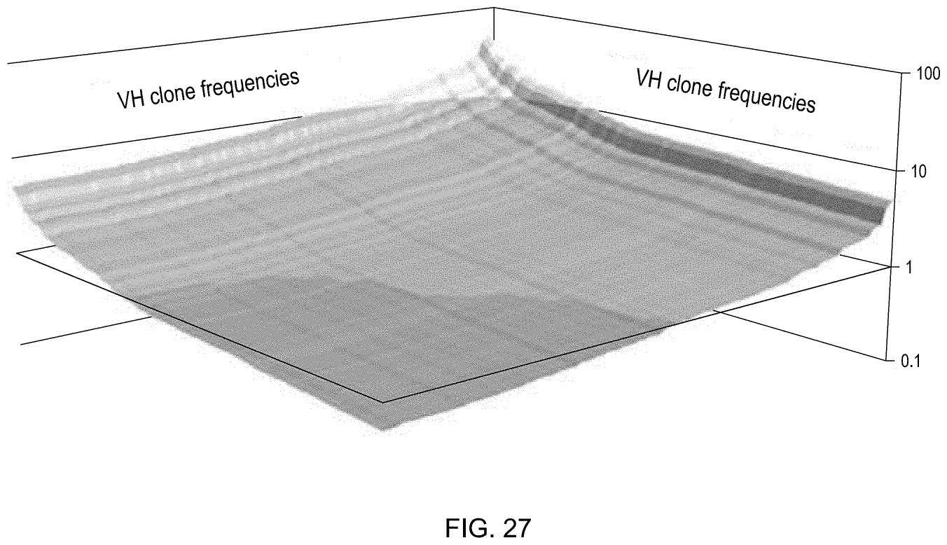

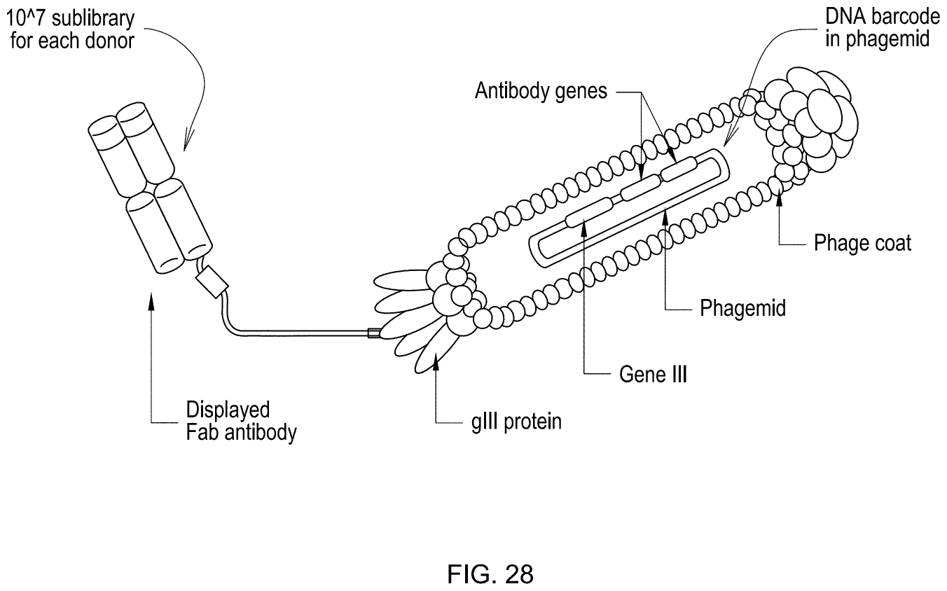

The method can comprise obtaining a sample comprising naïve B-cells and memory B-cells from a plurality of individuals. The sample can be a blood, plasma, or serum. A peripheral sample of human blood can comprise a few hundred thousand memory clones and plasmablasts, with a set of less than 10,000 that dominate the sample (FIG. 27). The plurality of individual can be a plurality of mammals. The plurality of mammals can be a plurality of primates, mice, rats, pigs, goats, rabbits, horses, cows, cats, or dogs. The plurality of mammals can be a plurality of humans. The plurality of individuals can be at least 25, 50, 75, 100, 125, or 150 individuals. The plurality of individuals can be between 50-100 individuals. The plurality of individuals can be between 50-140 individuals. The plurality of individuals can be at least 50 individuals. The plurality of individuals can be at least 140 individuals. A sample comprising naïve B-cells and memory B-cells from an individual can comprise at least about 5×10⁷ naïve B-cell clones and 5×10⁵ memory B-cell clones.

The method can comprise sorting or isolating the naïve B-cells from memory B-cells in the sample prior to obtaining the sequence information. The memory B-cells can be CD27+ B-cells. The sequence information can thus comprise separate sequence information for naïve B-cells and memory B-cells. Sorting naïve B-cells and memory B-cells can comprise the use of flow cytometry. In some cases, the flow cytometry is fluorescence-activated cell sorting (FACS). Sorting naïve B-cells and memory B-cells can comprise immunomagnetic cell separation procedures based on the markers present on the naïve B-cell or memory B-cell surface. Sorting the naïve B-cells and memory B-cells from the sample can produce a naïve B-cell pool and memory B-cell pool. Sorting the naïve B-cells and memory B-cells from the sample can produce a plurality of naïve B-cell pools and a plurality of memory B-cell pool. A naïve B-cell pool can comprise less than 0.1%, 1%, 5%, 10%, 20%, or 30% cells not of naïve B-cell origin. A naïve B-cell pool comprising less than 0.1%, 1%, 5%, 10%, 20%, or 30% cells not of naïve B-cell origin can also be referred to herein as a pool of predominantly naïve B-cell origin. A memory B-cell pool can comprise less than 0.1%, 1%, 5%, 10%, 20%, or 30% cells not of memory B-cell origin. A memory B-cell pool comprising less than 0.1%, 1%, 5%, 10%, 20%, or 30% cells not of memory B-cell origin can also be referred to herein as a pool of predominantly memory B-cell origin.

In some cases, a memory B-cell pool or a naïve B-cell pool is checked for quality using next generation sequencing (NGS). Pools with problematic diversity or biochemical liabilities can be rejected. Examples of biochemical liabilities include, but are not limited to, N-linked glycosylation, deamination, acid hydrolysis, positive charge endopeptidic cleavage, free cysteines, free methionines, alternative stop codons, cryptic splice sites, tev cleavage sites, and overly positively charged CDRs. In some cases, sequence data from at least one individual is removed from the pool. Sequence data from an individual can be removed from the pool if the sequence data has problematic diversity or biochemical liabilities.

The method can comprise extracting nucleic acid from naïve B-cells and extracting nucleic acid from memory B-cells. Nucleic acid can be extracted from each of the naïve cells and the memory cells after the naïve cells and the memory cells have been separated or isolated from the sample. The nucleic acid can be DNA or messenger RNA (mRNA). If the nucleic acid is mRNA, then the method can further comprise reverse transcribing the mRNA to complementary DNA (cDNA).

The method of preparing an antibody library can comprise obtaining sequence information for a plurality VH-CDR3 and VL-CDR3 sequences from naïve B-cells and sequence information for a plurality of VH-CDR1, VH-CDR2, VH-CDR3, VL-CDR1, VL-CDR2, and VL-CDR3 sequences from memory B-cells from a plurality of individuals. The sequence information from naïve B-cells can be obtained from a naïve B-cells pool. The sequence information from memory B-cells can be obtained from a memory B-cell pool. Obtaining the sequence information for the CDR sequences can comprising sequencing the CDR sequence. Sequencing the plurality of CDR sequences can comprise any suitable sequencing technology, for example next-generation sequencing (NGS) or Sanger sequencing. Examples of next-generation sequencing include, but are not limited to, pyrosequencing, sequencing-by-synthesis, sequencing-by-ligation, and single molecule sequencing. Sequencing the plurality of CDR sequences can produce the sequence information.

Sequencing the plurality of VH-CDRs and VL-CDRs sequences can comprise separately sequencing the nucleic acid extracted from the naïve B-cells and the nucleic acid extracted from the memory B-cells from sample.

Assembling, or synthesizing, the VH sequence or the VL sequence can comprise the use of overlap extension PCR (OE-PCR). In some cases, overlapping fragments comprising a portion of a CDR of the VH domain or CDR of the VL domain are generated. A plurality of overlapping fragments comprising a portion of the CDR of the VH domain or CDR of the VL domain can cover the entirety of the CDR of the VH domain sequence or CDR of the VL domain sequence. The overlapping fragments can be dsDNA fragments. OE-PCR can comprise assembly of the overlapping fragments to produce an entire CDR of the VH domain or CDR of the VL domain. The CDR of the VH domain can be VH-CDR1, VH-CDR2, VH-CDR3, or a combination thereof. The CDR of the VL domain can be VL-CDR1, VL-CDR2, VL-CDR3, or a combination thereof. VH-CDR1, VH-CDR2, VL-CDR1, VL-CDR2, VL-CDR3, or a combination thereof can be synthesized to contain at least one of the following characteristics: (a) are a CDR sequence derived from human germline sequences, (b) contain no more than 4 amino acid mutations when compared to the germline sequences, (c) are: (i) identified as naturally occurring in at least 2 individuals and enriched without a fitness disadvantage or (ii) are heavily enriched during panning; (d) do not contain any biochemical liabilities; or (e) a combination thereof. The germline sequence can be IGHJ4, IGHV1-69, IGHV1-46, IGHV3-23, IGKV1-39, IGKV2-28, IGKV3-15, or IGKV4-1, or combinations thereof.

The VH sequence, VL sequence, or combination thereof assembled using OE-PCR can be cloned into a vector. The vector can be a phage. The phage can be a bacteriophage, or a phagemid. The vector can be a HuCAL phage. The vector can further comprise a gene encoding a surface coat protein. The surface coat protein can be a pIII, pVIII, pVI, pVII, pIX, or gIII protein. The surface coat protein can be a gIII protein. In some instances, expression of the antibody encoded by the vector comprises expression of the antibody fused to the surface coat protein of the vector. The expressed antibody can be displayed on the surface of the vector.

The method of preparing an antibody library can comprise assembling a plurality of VL domain sequences, each VL domain sequence of the plurality of VL domain sequences comprising: a VL-CDR1 sequence derived from the sequence information from memory B-cells, a VL-CDR2 sequence derived from the sequence information from memory B-cells, and a VL-CDR3 sequence derived from the sequence information from memory B-cells or naïve B-cells. The VL domain sequence can be cloned into the vector in combination with a single fixed heavy chain sequence. The single fixed heavy chain sequence can be IGHV3-23 or IGHJ4. The single fixed heavy chain sequence can be referred to as a stuffer sequence.

The method of preparing an antibody library can comprise assembling a plurality of first nucleic acid sequences encoding a plurality of first antibodies, each first antibody of the plurality of first antibodies comprising: a variable light (VL) domain sequence; and a single fixed heavy chain sequence. The method of preparing an antibody library can comprise inserting the plurality of first nucleic acid sequences into a plurality of vectors. The vectors can be phages. The antibody encoded by the vector comprising the assembled VL domain and the single fixed heavy chain sequence can be expressed in the vector. The antibody can be expressed on the surface of the vector.

The method of preparing an antibody library can comprise applying at least one selective pressure to the plurality of vectors, wherein each vector in the plurality of vectors expresses an antibody. Following application of the selective pressure, a subset of phages able to withstand the selective pressure can be produced. The selective pressure can be application of a heat stress, selection with protein A, selection with protein L, or a combination thereof. The heat stress can be a temperature of about 65° C. The heat stress can be a temperature of at least 30° C., 40° C., 50° C., 60° C., 70° C., or 80° C. In some cases, applying a heat stress to the vector eliminates the vector if the vector is unstable or aggregation prone. In some cases, applying selection with protein A or protein L eliminates the vector if the vector expresses an antibody without the ability to bind to a protein. Applying selection with protein A or protein L can allow selection of antibodies which bind to proteins. The antibodies which bind to proteins can be thermostable. In some cases, after antibodies which bind to proteins are selected, the nucleotide sequences corresponding to the antibodies which bind to proteins can be determined.

The method of preparing an antibody library can comprise assembling a plurality of VH domain sequences, wherein each VH domains sequence comprising: a VH-CDR1 sequence derived from the sequence information from memory B-cells, a VH-CDR2 sequence derived from the sequence information from memory B-cells, and a VH-CDR3 sequence derived from the sequence information from memory B-cells or naïve B-cells. The VH-CDR1 sequence and a VH-CDR2 sequence can be a sequence obtained from memory B-cells, and the VH-CDR3 sequence can be a sequence from naïve B-cells. If the vector is successfully able to survive the selective pressures applied, the single fixed heavy chain sequence can be replaced with the assembled VH domain sequence. For each antibody in the plurality of antibodies, at least one of the VH-CDR3 sequence and the VL-CDR3 sequence can be derived from the sequence information from naïve B-cells

A vector comprising an assembled VL domain and an assembled VH domain can be transformed into a microbe. The microbe can be a bacteria. The bacteria can be a filamentous bacteria. The filamentous bacteria can be Escherichia coli. The microbe can be any suitable commercially available strain.

The vector can be transformed into a microbe using electroporation, chemical transformation, heat shock transformation, or a combination thereof.

Electroporation can comprise administering a high-voltage electric field to a ligation mixture comprising the microbes to be transformed and the vector. The high-voltage can range from 1 to 25 kV/cm. The high-voltage can range from 3 to 24 kV/cm. Examples of high voltage that can be administered to the microbes to induce transformation include, but are not limited to, 10 kV/cm, 15 kV/cm, 20 kV/cm, and 25 kV/cm. The high-voltage can be administered as a pulse or as a plurality of pulses. The plurality of pulses can be a pulse of high-voltage administered every 1, 2, 3, 4, 5, 6, 7, 8, 9, 10, 100, 500, or 1000 microseconds (μs). The plurality of pulses can be a pulse of high-voltage administered every 10, 20, 30, 30, 50, 60, 70 80, 90, or 100 milliseconds (msec). Electroporation can be administered to the microbes at room temperature or at 4° C.

Prior to addition to the ligation mixture, the vector can be purified and resuspended in water or TE. The ligation mixture can comprise a buffer. Examples of buffer include, but are not limited to, phosphate buffered saline (PBS), hepes buffer (HBSS), or a culture media. The buffer can be a hypoosmolar buffer. The buffer can be a high resistance buffer. In some cases, a recovery medium is added to the ligation mixture after electroporation.

Chemical transformation can comprise incubation of the microbe and the vector with a cation. The cation can be Mg2+, Mn2+, Rb+, or Ca2+. Chemical transformation can comprise incubation of the microbe and the vector with CaCl₂, MgCl₂, MnCl₂, or RbCl.

Heat shock transformation can comprise applying a high temperature to the microbes and the vector to induce transduction. The high temperature can be 42° C. The temperature can be applied for 10 seconds, 20 seconds, 30 seconds, 40 seconds, 50 seconds, or 1 minute. Heat shock can be applied before, during, or after electroporation or chemical transformation.

The vector can comprise a selectable marker. The selectable marker can be an antibiotic resistance gene or an optical selectable marker such as a green fluorescent protein. The antibiotic resistance gene can confer resistance of a microbe transformed with the vector to an antibody selected from the group consisting of: kanamycin, spectinomycin, streptomycin, ampicillin, carbenicillin, bleomycin, erthyromycin, polymxin B, tetracycline, chloramphenicol, and a combination thereof. The selectable marker can allow microbes that are not transformed with the vector to be eliminated.

A microbe transformed with a vector can be referred to as a transformant. Generating a plurality of antibodies can comprise the generation of a plurality of transformants. In some cases, the plurality of transformants comprises at least 7.6×10¹⁰ transformants. The at least 7.6×10¹⁰ transformants can comprise 0.5×10¹⁰ VH-CDR3 sequences. In some instances, at least 20% of the plurality of transformants are unique. For example, if the plurality of transformants comprises 7.6×10¹⁰ transformants, then in some embodiments at least 1.52×10¹⁰ of the transformants are unique. A unique transformant can be a transformant with a unique, or non-redundant, sequence compared to the other transformants in the plurality of transformants. In some cases, the antibody libraries described herein can be screened for antibodies with specificity to any desired target. Examples of targets include, but are not limited to, PD1, LAG3, OX40, CTLA4, SIRPa, CD47, VISTA, 41BB, TIM3, GITR, ICOS, TIGIT, GHR, HGH, amyloid beta, alpha synuclein, Tau, and beta secretase. The length of time to develop the antibody libraries described herein can be less than 2 months, less than 1 month, or less than 2 weeks. In one example, development of an antibody library can be less than 2 months, including time involved in panning, screening, and optimization (FIG. 2). In another example, the length of time can take less than 2 weeks (FIG. 2).

### Antibody Optimization and Resulting Libraries (Tumbler Libraries)

In some aspects, antibody libraries described herein may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) a CDR sequence selected from the group consisting of: a VH-CDR1 sequence, a VH-CDR2 sequence, a VH-CDR3 sequence, a VL-CDR1 sequence, a VL-CDR2 sequence, and a VL-CDR3 sequence, wherein the CDR sequence is the same for each antibody of the plurality of antibodies; and (d) a unique combination of remaining CDR sequences selected from the group consisting of: a VH-CDR1 sequence, a VH-CDR2 sequence, a VH-CDR3 sequence, a VL-CDR1 sequence, a VL-CDR2 sequence, and a VL-CDR3. The antibody library can also be referred to herein as a Tumbler Library.

In one example, an antibody library described herein may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VH-CDR1 sequence is the same for each antibody of the plurality of antibodies; and (d) the VH-CDR2 sequence, the VH-CDR3 sequence, the VL-CDR1 sequence, the VL-CDR2 sequence, and the VL-CDR3 are present in each antibody of the plurality of antibodies in a different combination. In some cases, the VH-CDR1 sequence is derived from an initial antibody clone.

In another example, an antibody library described herein may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VH-CDR2 sequence is the same for each antibody of the plurality of antibodies; and (d) the VH-CDR1 sequence, the VH-CDR3 sequence, the VL-CDR1 sequence, the VL-CDR2 sequence, and the VL-CDR3 are present in each antibody of the plurality of antibodies in a different combination. In some cases, the VH-CDR2 sequence is derived from an initial antibody clone.

In another example, an antibody library described herein may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VH-CDR3 sequence is the same for each antibody of the plurality of antibodies; and (d) the VH-CDR1 sequence, the VH-CDR2 sequence, the VL-CDR1 sequence, the VL-CDR2 sequence, and the VL-CDR3 are present in each antibody of the plurality of antibodies in a different combination. In some cases, the VH-CDR3 sequence is derived from an initial antibody clone.

In another example, an antibody library described herein may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VL-CDR1 sequence is the same for each antibody of the plurality of antibodies; and (d) the VH-CDR1 sequence, the VH-CDR2 sequence, the VH-CDR3 sequence, the VL-CDR2 sequence, and the VL-CDR3 sequence are present in each antibody of the plurality of antibodies in a different combination. In some cases, the VL-CDR1 sequence is derived from an initial antibody clone.

In another example, an antibody library described herein may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VL-CDR2 sequence is the same for each antibody of the plurality of antibodies; and (d) the VH-CDR1 sequence, the VH-CDR2 sequence, the VH-CDR3 sequence, the VL-CDR1 sequence, and the VL-CDR3 sequence are present in each antibody of the plurality of antibodies in a different combination. In some cases, the VL-CDR2 sequence is derived from an initial antibody clone.

In another example, an antibody library described herein may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VL-CDR3 sequence is the same for each antibody of the plurality of antibodies; and (d) the VH-CDR1 sequence, the VH-CDR2 sequence, the VH-CDR3 sequence, the VL-CDR1 sequence, and the VL-CDR2 sequence are present in each antibody of the plurality of antibodies in a different combination. In some cases, the VL-CDR3 sequence is derived from an initial antibody clone.

In various aspects, each antibody of the antibody library comprises a VH-CDR1 sequence, a VH-CDR2 sequence, a VH-CDR3 sequence, a VL-CDR1 sequence, a VL-CDR2 sequence, or a VL-CDR3 sequence selected from an initial antibody clone. The term “initial antibody clone” as used herein may refer to any antibody or antibody fragment with a desired property, such as affinity for a desired epitope, an amino acid sequence of said antibody or antibody fragment, a nucleotide sequence that encodes said antibody or antibody fragment, or any in silico amino acid or nucleotide sequence that corresponds to said antibody or antibody fragment. In various aspects, each antibody of the antibody library may comprise the same CDR sequence derived from an initial antibody clone. Additionally, each antibody of the antibody library may comprise a different combination of the remaining CDR sequences not derived from the initial antibody clone. In some cases, the remaining CDR sequences may be derived from a highly diverse antibody library (e.g., a SuperHuman antibody library, as described herein). In some cases, one of the CDRs may be derived from an initial antibody clone and the remaining CDRs may be derived from a highly diverse antibody library (e.g., a SuperHuman antibody library). In some cases, the highly diverse antibody library may have high diversity in each CDR sequence not derived from the initial antibody clone. In a non-limiting example, as depicted in FIG. 29, each antibody in an antibody library may comprise the same VH-CDR3 sequence derived from an initial antibody clone (“initial clone” in FIG. 29). Additionally, each antibody in the antibody library may comprise a different combination of the remaining CDR sequences that are not derived from the initial antibody clone (in this example, VH-CDR1, VH-CDR2, VL-CDR1, VL-CDR2, and VL-CDR3).

In various aspects, one or more of the VH-CDR1 sequence, the VH-CDR2 sequence, the VH-CDR3 sequence, the VL-CDR1 sequence, the VL-CDR2 sequence, and the VL-CDR3 sequence are naturally occurring. In various aspects, each of the VH-CDR1 sequence, the VH-CDR2 sequence, the VH-CDR3 sequence, the VL-CDR1 sequence, the VL-CDR2 sequence, and the VL-CDR3 sequence are naturally occurring, but are present in each antibody in non-naturally occurring combinations. In various aspects, one or more of the VH-CDR1 sequence, the VH-CDR2 sequence, the VH-CDR3 sequence, the VL-CDR1 sequence, the VL-CDR2 sequence, and the VL-CDR3 sequence are naturally occurring in a human population, or are derived from a human CDR sequence. In various aspects, each of the VH-CDR1 sequence, the VH-CDR2 sequence, the VH-CDR3 sequence, the VL-CDR1 sequence, the VL-CDR2 sequence, and the VL-CDR3 sequence are naturally occurring in a human population, or are derived from a human CDR sequence.

In various aspects, the antibodies of the library can comprise non-naturally occurring combinations of naturally occurring CDRs, such as combinations of CDRs derived from naturally occurring memory B-cells and naïve B-cells, but whose joint appearance on the same antibody would not be naturally occurring. For example, a non-naturally occurring combination of naturally occurring CDRs can comprise at least one CDR derived from a naïve cell while the remaining CDRs can be derived from a memory cell. For example, a non-naturally occurring combination of naturally occurring CDRs can comprise at least one CDR derived from cells of predominantly naïve B-cell origin while the remaining CDRs can be derived from cells of predominantly memory B-cell origin.

The non-naturally occurring combination of naturally occurring CDRs can comprise at least one CDR derived from a naïve cell, while the remaining CDRs are derived from a memory cell. In some cases, at least VL-CDR1 is derived from a naïve cell. In some cases, at least VL-CDR2 is derived from a naïve cell. In some cases, at least VL-CDR3 is derived from a naïve cell. In some cases, at least VH-CDR1 is derived from a naïve cell. In some cases, at least VH-CDR2 is derived from a naïve cell. In some cases, at least VH-CDR3 is derived from a naïve cell.

The non-naturally occurring combination of naturally occurring CDRs can comprise two, three, four, or five CDRs derived from a naïve cell, while the remaining CDRs can be derived from a memory cell. For example, two CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, three CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, four CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, five CDRs from CDRs in the group consisting of: VL-CDR1, VL-CDR2, VL-CDR3, VH-CDR1, VH-CDR2, and VH-CDR3 can be derived from a naïve cell while the remaining CDR can be derived from a memory cell.

In another non-limiting example of a non-naturally occurring combination, VL-CDR3 can be derived from a naïve cell, while VH-CDR1, VH-CDR2, VH-CDR3, VL-CDR1, and VL-CDR2 can be derived from a memory cell. In another non-limiting example of a non-naturally occurring combination, VH-CDR3 can be derived from a naïve cell, while VH-CDR1, VH-CDR2, VL-CDR1, VL-CDR2, and VL-CDR3 can be derived from a memory cell. In another non-limiting example of a non-naturally occurring combination, VH-CDR3 and VL-CDR3 can be derived from a naïve cell, while VH-CDR1, VH-CDR2, VL-CDR1, and VL-CDR2 can be derived from a memory cell.

A VH-CDR1 sequence derived from a naïve B-cell can comprise at least 80%, 85%, 90%, 95%, or 99% sequence homology to a naturally occurring VH-CDR1 sequence from a naïve B-cell. A VH-CDR1 sequence derived from a naïve B-cell can be a synthetic VH-CDR1 sequence. A VH-CDR1 sequence derived from a naïve B-cell can comprise 100% sequence homology to a naturally occurring VH-CDR1 sequence from a naïve B-cell. A VH-CDR2 sequence derived from a naïve B-cell can comprise at least 80%, 85%, 90%, 95%, or 99% sequence homology to a naturally occurring VH-CDR2 sequence from a naïve B-cell. A VH-CDR2 sequence derived from a naïve B-cell can be a synthetic VH-CDR2 sequence. A VH-CDR2 sequence derived from a naïve B-cell can comprise 100% sequence homology to a naturally occurring VH-CDR2 sequence from a naïve B-cell. A VH-CDR3 sequence derived from a naïve B-cell can comprise at least 80%, 85%, 90%, 95%, or 99% sequence homology to a naturally occurring VH-CDR3 sequence from a naïve B-cell. A VH-CDR3 sequence derived from a naïve B-cell can be a synthetic VH-CDR3 sequence. A VH-CDR3 sequence derived from a naïve B-cell can comprise 100% sequence homology to a naturally occurring VH-CDR3 sequence from a naïve B-cell.

A VL-CDR1 sequence derived from a naïve B-cell can comprise at least 80%, 85%, 90%, 95%, or 99% sequence homology to a naturally occurring VL-CDR1 sequence from a naïve B-cell. A VL-CDR1 sequence derived from a naïve B-cell can be a synthetic VL-CDR1 sequence. A VL-CDR1 sequence derived from a naïve B-cell can comprise 100% sequence homology to a naturally occurring VL-CDR1 sequence from a naïve B-cell. A VL-CDR2 sequence derived from a naïve B-cell can comprise at least 80%, 85%, 90%, 95%, or 99% sequence homology to a naturally occurring VL-CDR2 sequence from a naïve B-cell. A VL-CDR2 sequence derived from a naïve B-cell can be a synthetic VL-CDR2 sequence. A VL-CDR2 sequence derived from a naïve B-cell can comprise 100% sequence homology to a naturally occurring VL-CDR2 sequence from a naïve B-cell. A VL-CDR3 sequence derived from a naïve B-cell can comprise at least 80%, 85%, 90%, 95%, or 99% sequence homology to a naturally occurring VL-CDR3 sequence from a naïve B-cell. A VL-CDR3 sequence derived from a naïve B-cell can be a synthetic VL-CDR3 sequence. A VL-CDR3 sequence derived from a naïve B-cell can comprise 100% sequence homology to a naturally occurring VL-CDR3 sequence from a naïve B-cell.

The VH-CDR1 sequence, VH-CDR2 sequence, VH-CDR3 sequence, VL-CDR1 sequence, VL-CDR2 sequence, VL-CDR3 sequence, or any combination thereof can be derived from sequence information obtained from a pool of cells of predominantly naïve B-cell origin. The VH-CDR3 sequence, the VL-CDR3 sequence, or the combination thereof can be derived from sequence information obtained from a pool of cells of predominantly naïve B-cell origin. The pool of naïve B-cells can be obtained from a plurality of individuals. The pool of naïve B-cells can comprise less than 0.1%, 1%, 5%, 10%, 20%, or 30% cells not of naïve B-cell origin.

A VH-CDR1 sequence derived from a memory B-cell can comprise at least 80%, 85%, 90%, 95%, or 99% sequence homology to a naturally occurring VH-CDR1 sequence from a memory B-cell. A VH-CDR1 sequence derived from a memory B-cell can be a synthetic VH-CDR1 sequence. A VH-CDR1 sequence derived from a memory B-cell can comprise 100% sequence homology to a naturally occurring VH-CDR1 sequence from a memory B-cell. A VH-CDR2 sequence derived from a memory B-cell can comprise at least 80%, 85%, 90%, 95%, or 99% sequence homology to a naturally occurring VH-CDR2 sequence from a memory B-cell. A VH-CDR2 sequence derived from a memory B-cell can be a synthetic VH-CDR2 sequence. A VH-CDR2 sequence derived from a memory B-cell can comprise 100% sequence homology to a naturally occurring VH-CDR2 sequence from a memory B-cell. A VH-CDR3 sequence derived from a memory B-cell can comprise at least 80%, 85%, 90%, 95%, or 99% sequence homology to a naturally occurring VH-CDR3 sequence from a memory B-cell. A VH-CDR3 sequence derived from a memory B-cell can be a synthetic VH-CDR3 sequence. A VH-CDR3 sequence derived from a memory B-cell can comprise 100% sequence homology to a naturally occurring VH-CDR3 sequence from a memory B-cell.

A VL-CDR1 sequence derived from a memory B-cell can comprise at least 80%, 85%, 90%, 95%, or 99% sequence homology to a naturally occurring VL-CDR1 sequence from a memory B-cell. A VL-CDR1 sequence derived from a memory B-cell can be a synthetic VH-CDR1 sequence. A VL-CDR1 sequence derived from a memory B-cell can comprise 100% sequence homology to a naturally occurring VL-CDR1 sequence from a memory B-cell. A VL-CDR2 sequence derived from a memory B-cell can comprise at least 80%, 85%, 90%, 95%, or 99% sequence homology to a naturally occurring VL-CDR2 sequence from a memory B-cell. A VL-CDR2 sequence derived from a memory B-cell can comprise 100% sequence homology to a naturally occurring VL-CDR2 sequence from a memory B-cell. A VL-CDR3 sequence derived from a memory B-cell can comprise at least 80%, 85%, 90%, 95%, or 99% sequence homology to a naturally occurring VL-CDR3 sequence from a memory B-cell. A VL-CDR3 sequence derived from a memory B-cell can comprise 100% sequence homology to a naturally occurring VL-CDR3 sequence from a memory B-cell.

The VH-CDR1 sequence, VH-CDR2 sequence, VH-CDR3 sequence, VL-CDR1 sequence, VL-CDR2 sequence, VL-CDR3 sequence, or any combination thereof can be derived from sequence information obtained from a pool of cells of predominantly memory B-cell origin. The VH-CDR3 sequence, the VL-CDR3 sequence, or the combination thereof can be derived from sequence information obtained from a pool of cells of predominantly memory B-cell origin. The pool of memory B-cells can be obtained from a plurality of individuals. The pool of memory B-cells can comprise less than 0.1%, 1%, 5%, 10%, 20%, or 30% cells not of memory B-cell origin. The memory B-cells can be CD27+ B-cells. The pool of memory B-cells can comprise less than 0.1%, 1%, 5%, 10%, 20%, or 30% cells not of CD27+ B-cell origin.

The naïve cell can be a naïve B-cell. The naïve B-cell can be a human naïve B-cell. The memory cell can be a memory B-cell. The memory B-cell can be a human memory B-cell. In some instances, a naïve B-cell shows increased diversity of VH-CDR3 and VL-CDR3 sequences compared to VH-CDR3 and VL-CDR3 sequences from a memory B-cell (FIG. 1). The naïve cells and the memory cells can be obtained from a biological sample, such as blood, from an individual or a plurality of individuals. The naïve cells and the memory cells can be physically isolated from this sample using a marker specific to the naïve cells or the memory cells.

A marker can be used to identify, separate, or sort B-cells, naïve B-cells, and memory B-cells from a biological sample. Examples of markers used to identify, separate, or sort a B-cell include, but are not limited to, CD19+. Examples of markers used to identify, separate, or sort a naïve B-cell include, but are not limited to, CD19+, CD27−, IgD+, IgM+, and combinations thereof. Examples of markers used to identify, separate, or sort a memory B-cell include, but are not limited to, CD19+, CD27+, and combinations thereof. In some embodiments, CD27+ is used to sort memory B-cells. Examples of markers used to identify, separate, or sort a class switched memory B-cell include, but are not limited to CD19+, CD27+, CD27+, IgD−, IgM−, and combinations thereof. Examples of markers used to identify, separate, or sort a nonswitched or marginal zone memory B-cell include, but are not limited to, CD19+, CD27+, IgD+, IgM+, and combinations thereof. In some cases, a memory B-cell can be identified, separated, or sorted with the following markers: CD19+, CD27+, IgD−, IgM+, and combinations thereof. The naïve cell from which the VH-CDR3 is derived can be a CD27−/IgM+ B-cell. The memory cell from which the VH-CDR1, VH-CDR2, VL-CDR1, VL-CDR2, and VL-CDR3 are derived can be a CD27+/IgG+ B-cell.

The CDR sequences of the antibody can be CDR sequences found in naïve B-cells and memory B-cells found in an individual or a plurality of individuals. The individual can be a mammal. The mammal can be a human, a non-human primate, mouse, rat, pig, goat, rabbit, horse, cow, cat, or dog. In some cases, the CDR sequences are CDR sequences obtained from a publically available source. Examples of publically available sources of CDR sequences include SAbDab (http://opig.stats.ox.ac.uk/webapps/sabdab-sabpred/Welcome.php) and PylgClassify (http://dunbrack2.fccc.edu/PyIgClassify/).

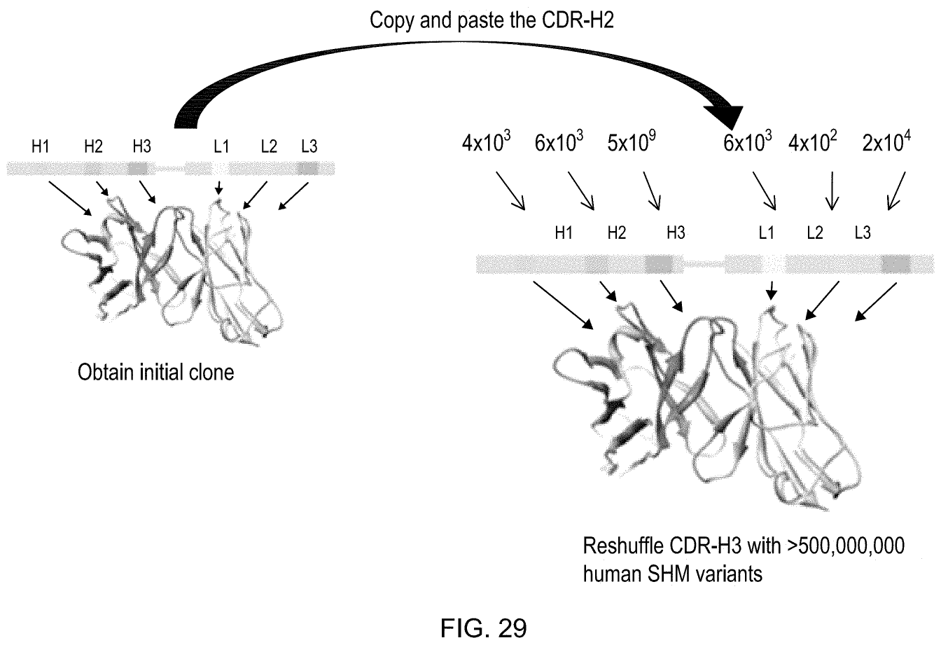

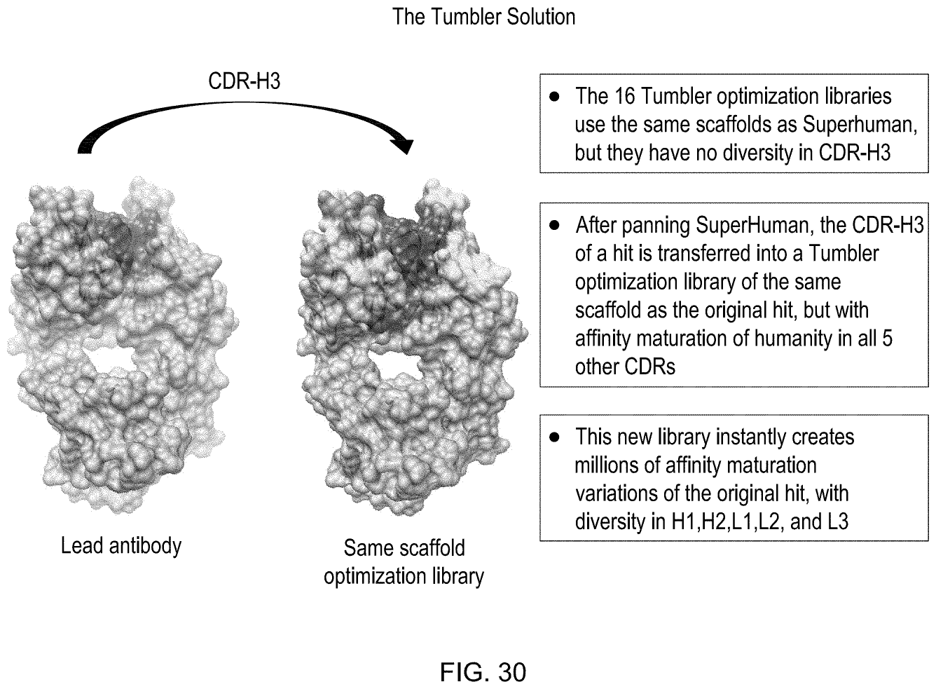

In various aspects, each antibody in the antibody library may comprise the same scaffold, for example, the same combination of framework sequences (see FIG. 30). In various aspects, the VH domain of each antibody of the antibody library comprises a VH-FR1 sequence, a VH-FR2 sequence, a VH-FR3 sequence, and a VH-FR4 sequence. In some cases, each of the VH-FR1 sequence, the VH-FR2 sequence, the VH-FR3 sequence, and the VH-FR4 sequence is the same for each antibody in the antibody library. In some cases, each of the VH-FR1 sequence, the VH-FR2 sequence, the VH-FR3 sequence, and the VH-FR4 sequence is derived from the initial antibody clone from which the CDR sequence is derived (see FIG. 30). In some cases, the VH-FR1 sequence may be the same VH-FR1 sequence as the initial antibody clone. In some cases, the VH-FR2 sequence may be the same VH-FR2 sequence as the initial antibody clone. In some cases, the VH-FR3 sequence may be the same VH-FR3 sequence as the initial antibody clone. In some cases, the VH-FR4 sequence may be the same VH-FR4 sequence as the initial antibody clone. In various aspects, the VL domain of each antibody of the antibody library comprises a VL-FR1 sequence, a VL-FR2 sequence, a VL-FR3 sequence, and a VL-FR4 sequence. In some cases, each of the VL-FR1 sequence, the VL-FR2 sequence, the VL-FR3 sequence, and the VL-FR4 sequence is the same for each antibody in the antibody library. In some cases, each of the VL-FR1 sequence, the VL-FR2 sequence, the VL-FR3 sequence, and the VL-FR4 sequence may be derived from the initial antibody clone from which the CDR sequence is derived (see FIG. 30). In some cases, the VL-FR1 sequence may be the same VL-FR1 sequence as the initial antibody clone. In some cases, the VL-FR2 sequence may be the same VL-FR2 sequence as the initial antibody clone. In some cases, the VL-FR3 sequence may be the same VL-FR3 sequence as the initial antibody clone. In some cases, the VL-FR4 sequence may be the same VL-FR4 sequence as the initial antibody clone.

The framework of the antibody can be a naturally occurring framework. The naturally occurring framework can be a framework found in a mammal. The mammal can be a primate, mouse, rat, pig, goat, rabbit, horse, cow, cat, or dog. The primate can be a human. The framework can comprise at least one variant compared to a naturally occurring framework. The variant can be a mutation, an insertion, or a deletion. The variant can be a variant found in the nucleic acid sequence encoding the antibody or a variant found in the amino acid sequence of the antibody. Any suitable framework sequence can be used, such as those previously used in phase I clinical trials (FIGS. 12A, 12B). As used herein, the framework of an antibody can refer to the framework regions of the variable heavy chain (VH-FR1, VH-FR2, VH-FR3, and VH-FR4), the framework regions of the variable heavy chain (VL-FR1, VL-FR2, VL-FR3, and VL-FR4), or a combination thereof. The framework regions of an antibody in the antibody library can be identical to germline framework regions.

The framework can be a therapeutically optimal framework. The therapeutically optimal framework can comprise at least one, at least two, at least three, at least four, at least five, or all of the following properties selected from the group consisting of: a) previously demonstrated safety in human monoclonal antibodies, b) thermostable; c) not prone to aggregation; d) comprises a single dominant allele at the amino acid level across all human populations; e) comprises different canonical topologies of the CDRs; f) expresses well in bacteria; and g) displays well on a phage. A framework with previously demonstrated safety in human monoclonal antibodies can be a framework of an antibody that has been used in at least a phase I clinical trial. A framework that is thermostable can be a framework that is thermostable at at least 20° C., 30° C., 40° C., 50° C., 60° C., 70° C., 80° C., 90° C., 100° C., or over 100° C. A framework that is thermostable can be a framework that can withstand a temperature increase of at least 3° C. per minute, 4° C. per minute, or 5° C. per minute. A framework that expresses well in bacteria can be a framework that produces a biologically active antibody in the bacteria. The bacteria can be E. coli. The bacteria can be an engineered bacteria. The bacteria can be a bacteria optimized for antibody expression. A framework that displays well on a phage can be a framework that produces a biologically active antibody when displayed on the surface of the phage.

An example of a strategy used to choose a framework is described in FIG. 11, wherein an ideal framework of an antibody can be one that shows structural diversity, has been used successfully in a Phase I clinical trial in humans, has low immunogenicity, shows aggregation resistance, displays fitness, and is thermostable. In some cases, frameworks of antibodies are avoided if they are inherently autoreactive to blood cells (e.g., IGHV4-34), have an inferior stability profile (e.g., IGHV2-5), have a V-gene not found in at least 50% of individuals (e.g., IGHV4-b), show an aggregation prone V-gene (e.g., IGLV6-57), or a combination thereof.

The amino acid sequence of a framework of an antibody herein can comprise more than one dominant allele, wherein there are different dominant alleles in different human populations (FIG. 13 and FIG. 14). For example, the IGHV1-3 framework comprises 3 alleles: IGVH1-3*01, IGVH1-3*02, and IGVH1-3*03, which are found in different frequencies in different human populations (FIG. 26). In some instances, the amino acid sequence of the framework of the antibodies described herein has a single dominant allele in at least two human populations. In some instances, the amino acid sequence of the framework of the antibodies described herein has a single dominant allele in all human populations. A framework with one dominant allele can be a framework in which one allele is found in at least 50%, at least 75%, or at least 90% in at least two human populations. A framework with one dominant allele can be a framework in which one allele is found in at least 50%, at least 75%, or at least 90% in at least twelve human populations. In some cases, the framework regions of the VH domain are framework regions from IGHJ4, IGHV1-46, IGHV1-69, IGHV3-15, or IGHV3-23. In some cases, the framework regions of the VH domain are framework regions from IGHV2-5, IGHV3-7, IGVH4-34, IGHV5-51, IGHV1-24, IGHV2-26, IGHV3-72, IGHV3-74, IGHV3-9, IGHV3-30, IGHV3-33, IGHV3-53, IGHV3-66, IGHV4-30-4, IGHV4-31, IGHV4-59, IGHV4-61, or IGHV5-51. In some cases, the framework regions of the VH domain of the antibodies in the antibody library are framework regions from IGHV1-46, IGHV3-23, or a combination thereof. In some cases, the frameworks regions of the VL domain of the antibodies in the antibody library are framework regions from IGKV1-39, IGKV2-28, IGKV3-15, IGKV4-1, IGKV1-5, IGKV1-12, IGKV1-13, IGKV3-11, IGKV3-20, or a combination thereof. In one example, a subset of antibodies in the antibody library can have framework regions of the VH domain from IGHV1-46 and framework regions of the VL domain from IGKV1-39, while the remaining antibodies in the antibody library have framework regions of the VH domain from IGHV1-46 and framework regions of the VL domain from IGKV2-28.

Disclosed herein, in some cases, are nucleic acid sequences encoding an antibody described herein. The nucleic acid sequence can be a DNA or an RNA sequence. The nucleic acid can be inserted into a vector. The vector can be a phage. The phage can be a phagemid or a bacteriophage. The phagemid can be pMID21. The bacteriophage can be DY3F63, an M13 phage, fd filamentous phage, T4 phage, T7 phage or λ phage. In some cases a phagemid can be introduced into a microbe in combination with a bacteriophage (e.g., a ‘helper’ phage). The microbe can be a filamentous bacteria. The filamentous bacteria can be Escherichia coli.

The antibody libraries described herein comprise a plurality of antibodies. The plurality of antibodies can be at least 1.0×10⁶, 1.0×10⁷, 1.0×10⁸, 1.0×10⁹, 1.0×10¹⁰, 2.0×10¹⁰, 3.0×10¹⁰, 4.0×10¹⁰, 5.0×10¹⁰, 6.0×10¹⁰, 7.0×10¹⁰, 8.0×10¹⁰, 9.0×10¹⁰, or 10.0×10¹⁰ antibodies. The plurality of antibodies can be at least 1.0×10¹¹ antibodies. The plurality of antibodies can be at least 7.6×10¹⁰ antibodies. Such libraries can be unique because of their high diversity. For example, in any of the libraries herein at least 2%, at least 5%, at least 10%, at least 15%, at least 20%, at least 25%, at least 30%, or at least 35% of the plurality of antibodies can be unique. In some instances, a library has more than 7.0×10¹⁰ antibodies of which at least 20% of the plurality of antibodies are unique. The unique antibody can vary by at least one nucleic acid or at least one amino acid residue relative to the other antibodies of the antibody library.

In some instances, the antibody library comprises at least 1.0×10⁵ antibodies of which at least 80% of the plurality of antibodies are functional (e.g., bind to a desired antigen with a K_{d} of less than 100 nM). In some instances, the antibody library comprises at least 7.0×10¹⁰ antibodies of which at least 80% of the plurality of antibodies are functional. In some instances, the antibody library comprises at least 7.6×10¹⁰ antibodies of which at least 80% of the plurality of antibodies are functional.

In some instances, the antibody library comprises at least 1.0×10⁵ antibodies of which at least 85% of the plurality of antibodies are functional. In some instances, the antibody library comprises at least 7.0×10¹⁰ antibodies of which at least 85% of the plurality of antibodies are functional. In some instances, the antibody library comprises at least 7.6×10¹⁰ antibodies of which at least 85% of the plurality of antibodies are functional.

In some instances, the antibody library comprises at least 1.0×10⁵ antibodies of which at least 90% of the plurality of antibodies are functional. In some instances, the antibody library comprises at least 7.0×10¹⁰ antibodies of which at least 90% of the plurality of antibodies are functional. In some instances, the antibody library comprises at least 7.6×10¹⁰ antibodies of which at least 90% of the plurality of antibodies are functional.

In some instances, the antibody library comprises at least 1.0×10⁵ antibodies of which at least 95% of the plurality of antibodies are functional. In some instances, the antibody library comprises at least 7.0×10¹⁰ antibodies of which at least 95% of the plurality of antibodies are functional. In some instances, the antibody library comprises at least 7.6×10¹⁰ antibodies of which at least 95% of the plurality of antibodies are functional.

In some instances, the antibody library comprises at least 1.0×10⁵ antibodies of which at least 99% of the plurality of antibodies are functional. In some instances, the antibody library comprises at least 7.0×10¹⁰ antibodies of which at least 99% of the plurality of antibodies are functional. In some instances, the antibody library comprises at least 7.6×10¹⁰ antibodies of which at least 99% of the plurality of antibodies are functional.

In various aspects, the antibody library may have high diversity in one or more CDR sequences. In some cases, the antibody library may have high diversity in a VH-CDR1 sequence. In some cases, the antibody library may have high diversity in a VH-CDR2 sequence. In some cases, the antibody library may have high diversity in a VH-CDR3 sequence. In some cases, the antibody library may have high diversity in a VL-CDR1 sequence. In some cases, the antibody library may have high diversity in a VL-CDR2 sequence. In some cases, the antibody library may have high diversity in a VL-CDR3 sequence. In some cases, the antibody library may have high diversity in a CDR sequence not derived from an initial antibody clone, and low or no diversity in the CDR sequence derived from the initial antibody clone. In some cases, an antibody library with high diversity may comprise at least 1×10³, 5×10³, 1×10⁴, 5×10⁴, 1×10⁵, 5×10⁵, 1×10⁶, 5×10⁶, or more unique CDR sequences. In some cases, an antibody library may comprise high diversity in five of the six CDR sequences, for example, an antibody library may comprise high diversity in five CDR sequences selected from the group consisting of: a VH-CDR1 sequence, a VH-CDR2 sequence, a VH-CDR3 sequence, a VL-CDR1 sequence, a VL-CDR2 sequence, and a VL-CDR3 sequence. In such cases, the remaining CDR sequence may have low or no diversity. In some cases, the remaining CDR sequence is the same CDR sequence for each antibody. In some cases, the remaining CDR sequence is derived from an initial antibody clone.

In various aspects, at least one antibody of the antibody library may exhibit an improvement in at least one characteristic as compared to an initial antibody clone. In some cases, at least one antibody of the antibody library may exhibit an improvement in thermal stability (e.g., higher Tm) as compared to an initial antibody clone. For example, at least one antibody of the antibody library may have a melting temperature (Tm) that is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or great than 50° C., higher than an initial antibody clone. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library may have a higher Tm than an initial antibody clone.

In some cases, at least one antibody of the antibody library may exhibit greater affinity (e.g., a lower dissociation constant (K_{d})) for a target epitope as compared to an initial antibody clone. For example, at least one antibody of the antibody library may have a dissociation constant (K_{d}) for a target epitope that is at least 5×, at least 10×, at least 20×, at least 30×, at least 40×, at least 50×, at least 60×, at least 70×, at least 80×, at least 90×, at least 100×, at least 200×, at least 300×, at least 400×, at least 500×, at least 600×, at least 700×, at least 800×, at least 900×, or at least 1000× lower than the K_{d} of initial antibody clone. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library may have a lower K_{d} than an initial antibody clone.

In some cases, at least one antibody of the antibody library may exhibit increased species selectivity as compared to an initial antibody clone. For example, at least one antibody of the antibody library may have a K_{d} for a target epitope of a specific species that is at least 5×, at least 10×, at least 20×, at least 30×, at least 40×, at least 50×, at least 60×, at least 70×, at least 80×, at least 90×, at least 100×, at least 200×, at least 300×, at least 400×, at least 500×, at least 600×, at least 700×, at least 800×, at least 900×, or at least 1000× lower than the K_{d} of an initial antibody clone. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library may have a lower K_{d} for an epitope from a specific species than an initial antibody clone.

In some cases, at least one antibody of the antibody library may exhibit increased species cross-reactivity (e.g., across primate species) as compared to an initial antibody clone. For example, at least one antibody of the antibody library may have a lower K_{d} for epitope A from species A (e.g., a cynomolgus monkey), and may have high affinity for an analogous epitope A′ from species B (e.g., a human). In some cases, at least one antibody of the antibody library may have a K_{d} for a target epitope of a first species that is at least 5×, at least 10×, at least 20×, at least 30×, at least 40×, at least 50×, at least 60×, at least 70×, at least 80×, at least 90×, at least 100×, at least 200×, at least 300×, at least 400×, at least 500×, at least 600×, at least 700×, at least 800×, at least 900×, or at least 1000× lower than the K_{d} of an initial antibody clone, and may also have a K_{d} for an analogous epitope of a second species that is at least 5×, at least 10×, at least 20×, at least 30×, at least 40×, at least 50×, at least 60×, at least 70×, at least 80×, at least 90×, at least 100×, at least 200×, at least 300×, at least 400×, at least 500×, at least 600×, at least 700×, at least 800×, at least 900×, or at least 1000× lower than the K_{d} of an initial antibody clone. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library may have a lower K_{d} for an epitope from a first species and an analogous epitope from a second species as compared to an initial antibody clone.

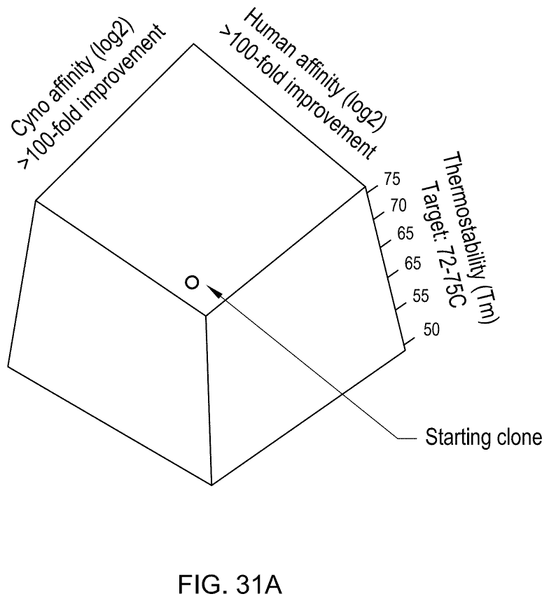

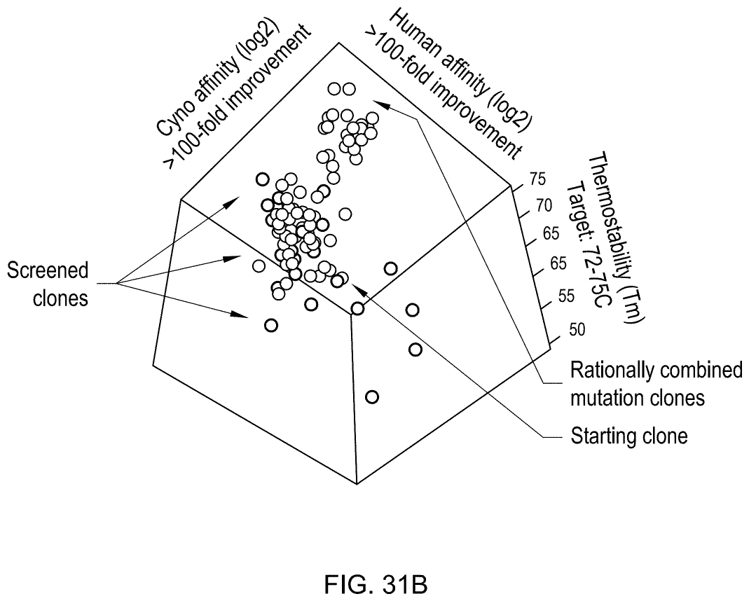

In various aspects, an antibody library may comprise one or more antibodies that exhibit an improvement in more than one characteristic as compared to an initial antibody clone. In some cases, the improvement is selected from the group consisting of: improved thermostability, improved affinity for a target epitope, improved selectivity for a target epitope of a specific species, and improved cross-reactivity across species. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library exhibit an improvement in two of the following: thermostability, affinity for a target epitope, selectivity for a target epitope of a specific species, or cross-reactivity across species. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library exhibit an improvement in three of the following: thermostability, affinity for a target epitope, selectivity for a target epitope of a specific species, or cross-reactivity across species. FIGS. 31A & B depict a non-limiting example of selecting for an antibody clone exhibiting improved thermal stability, improved affinity for epitope A from cynomolgus monkey, and improved affinity for epitope A from human.

In various aspects, an antibody of the antibody library may exhibit thermal stability. In some cases, an antibody of the antibody library may have a melting temperature (Tm) that is between about 50° C. and about 90° C. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library have a melting temperature (Tm) that is between about 50° C. and about 90° C. For example, an antibody of the antibody library may have a melting temperature (Tm) of at least 50° C., 55° C., 60° C., 65° C., 70° C., 75° C., 80° C., 85° C., or 90° C.

In various aspects, an antibody of the antibody library may exhibit high affinity for a target epitope. In some cases, at least 5%, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or greater than 95% of the antibodies in an antibody library may exhibit high affinity for a target epitope. For example, an antibody of the antibody library may bind to a target epitope with a dissociation constant (K_{d}) of less than about 50 nM, 25 nM, 10 nM, 5 nM, 1 nM, 900 pM, 800 pM, 700 pM, 600 pM, 500 pM, 400 pM, 300 pM, 200 pM, 100 pM, 50 pM, 25 pM, 10 pM, 5 pM, 1 pM, 900 fM, 800 fM, 700 fM, 600 fM, 500 fM, 400 fM, 300 fM, 200 fM, 100 fM, 50 fM, 25 fM, 10 fM, 5 fM, 1 fM, or less.

### Methods of Generating Antibody Libraries Using Tumbler

In one aspect, a method is provided for generating an antibody library, such as an antibody library described above. In some cases, the methods comprise: (a) selecting a CDR sequence, wherein the CDR sequence is selected from the group consisting of: a VH-CDR1 sequence, a VH-CDR2 sequence, a VH-CDR3 sequence, a VL-CDR1 sequence, a VL-CDR2 sequence, and a VL-CDR3 sequence; (b) replacing a CDR sequence for each antibody of a first antibody library with the CDR sequence selected in (a), thereby generating a second antibody library comprising a plurality of antibodies, wherein each antibody of the plurality of antibodies comprises: (i) the CDR sequence selected in (a); and (ii) a unique combination of remaining CDR sequences not selected in (a), wherein the remaining CDR sequences are selected from the group consisting of: a VH-CDR1 sequence, a VH-CDR sequence, a VH-CDR3 sequence, a VL-CDR1 sequence, a VL-CDR2 sequence, and a VL-CDR3 sequence.

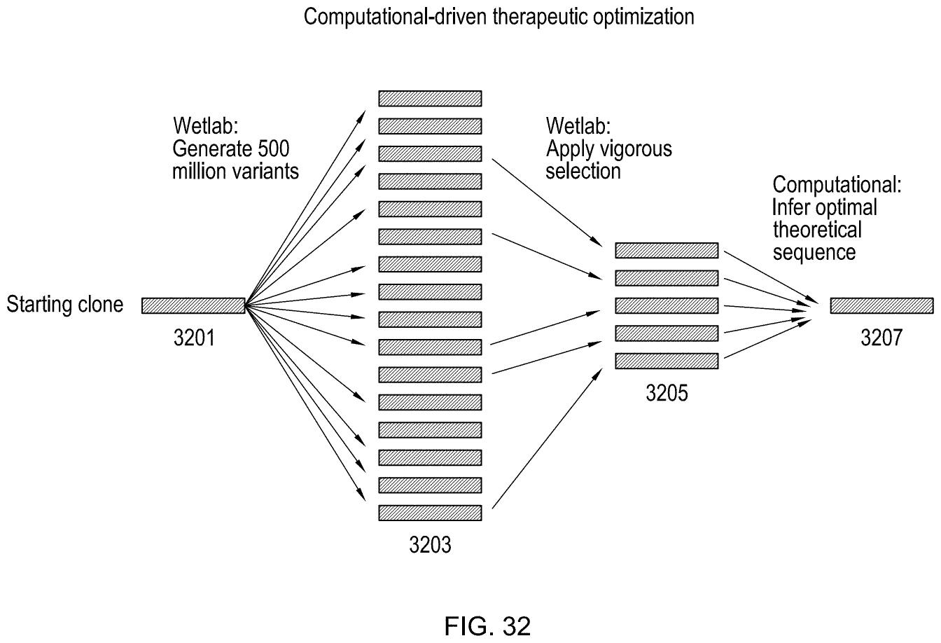

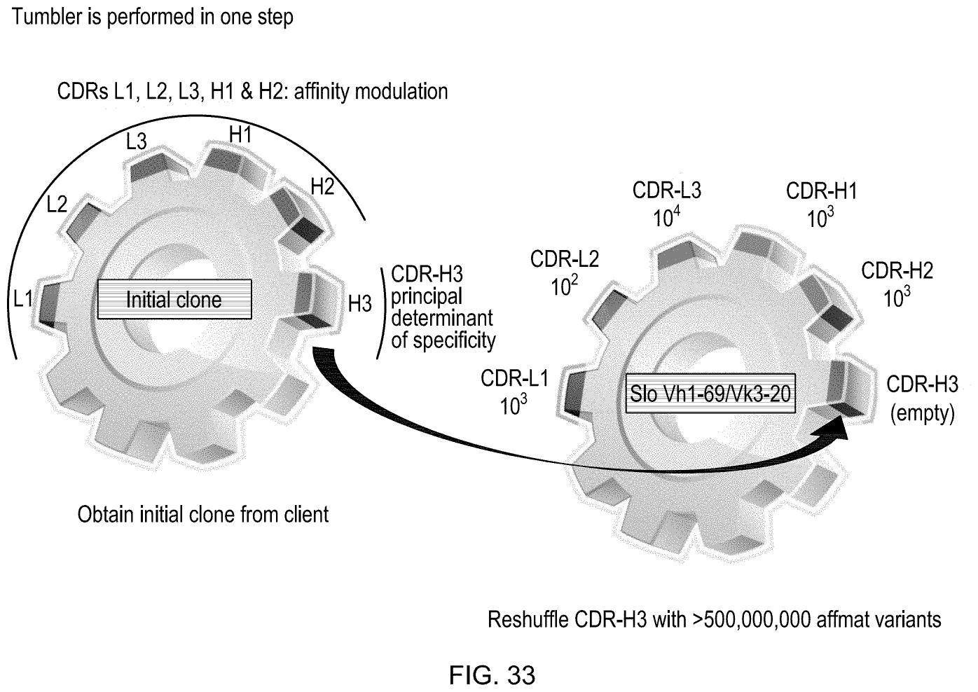

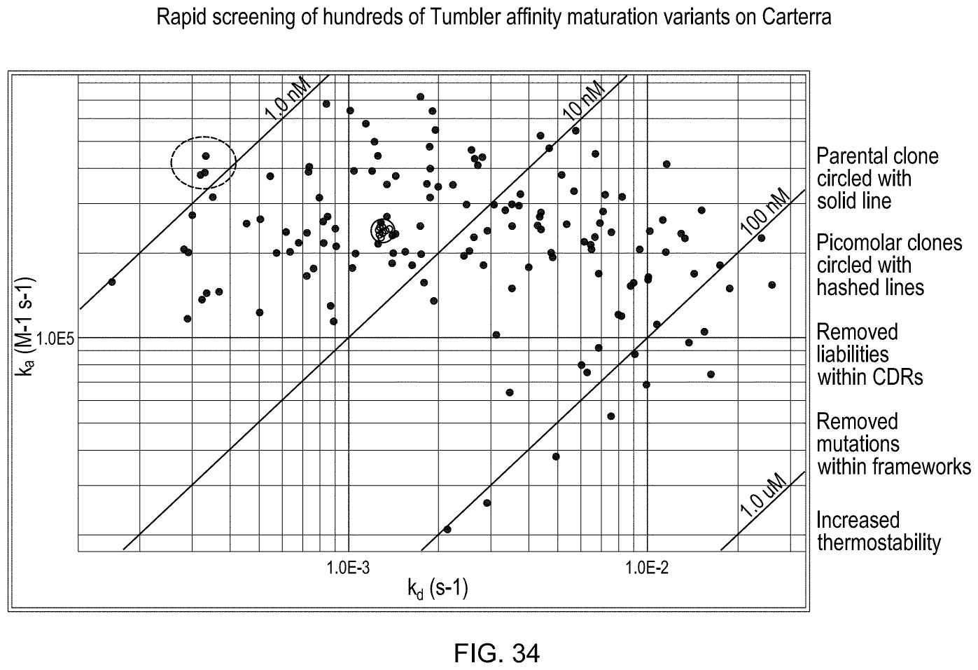

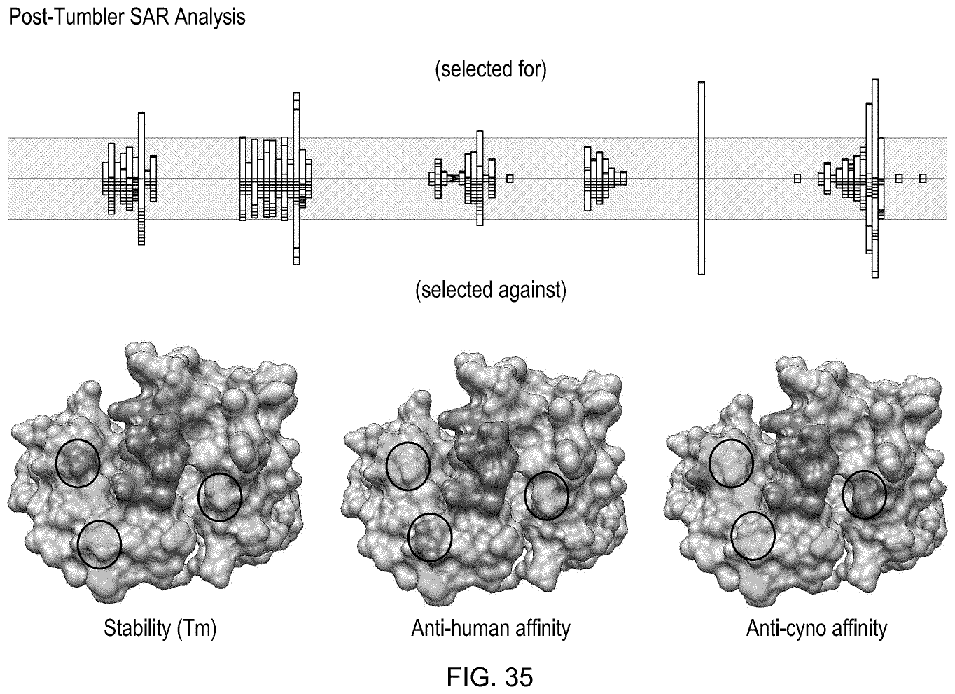

FIG. 32 and FIG. 33 depict a non-limiting example workflow of methods of generating antibody libraries, and obtaining one or more desired antibodies therefrom. In some cases, an initial antibody clone is obtained (FIG. 32, 3201). In some cases, the initial antibody clone may be obtained from a third party, such as a customer or client. In other cases, the initial antibody clone may be obtained from a highly diverse antibody library (e.g., a SuperHuman antibody library, as described herein). In some cases, the initial antibody clone may have a desired property, such as affinity for a specific epitope. In some cases, it may be desirous to improve one or more characteristics of the initial antibody clone. For example, it may be desirous to improve the thermostability (e.g., increase the melting temperature (Tm)) of the antibody, the binding characteristics of the antibody (e.g., affinity), or the species cross-reactivity of the antibody across two or more species. The initial antibody clone may then be used to generate an antibody library (FIG. 32, 3203). In some cases, a CDR sequence from the initial antibody clone may be selected. The CDR sequence may be any one of: a VH-CDR1 sequence, a VH-CDR2 sequence, a VH-CDR3 sequence, a VL-CDR1 sequence, a VL-CDR2 sequence, or a VL-CDR3 sequence. In particular aspects, the CDR sequence is a VH-CDR3 sequence (see FIG. 33). Generally, the CDR sequence selected from the initial antibody clone is a CDR sequence that is important for a desired characteristic, such as a CDR sequence important for binding affinity for a target epitope. In various aspects, the CDR sequence selected from the initial antibody clone may be cloned into a highly diverse antibody library. In some cases, the highly diverse antibody library may have high diversity in five of the six CDR sequences, and little to no diversity in the one CDR sequence being replaced (see, e.g., FIG. 30 and FIG. 33). In some cases, the highly diverse antibody library may be a SuperHuman antibody library or a modified SuperHuman antibody library (e.g., same scaffold as a SuperHuman antibody library, but with no diversity in the CDR sequence being replaced; see FIG. 33). In some cases, a CDR sequence of each antibody of the highly diverse antibody library may be replaced with the CDR sequence selected from the initial antibody clone, such that each antibody in the subsequent antibody library has the same CDR sequence. For example, the VH-CDR3 sequence from an initial antibody clone may be cloned into a highly diverse antibody library, such that each VH-CDR3 sequence of the highly diverse antibody library is replaced by the same VH-CDR3 sequence selected from the initial antibody clone (see FIG. 33). In such cases, the remaining CDR sequences (in this example, VH-CDR1, VH-CDR2, VL-CDR1, VL-CDR2, and VL-CDR3) are the CDR sequences present in the highly diverse antibody library. In some cases, a CDR sequence may be cloned into a highly diverse antibody library using a method that introduces mutations into the CDR sequence (e.g., to introduce more diversity into the CDR sequence). In some examples, the CDR sequence may be cloned by performing an error-prone PCR method to introduce one or more mutations into the CDR sequence. In some cases, each antibody of the highly diverse antibody library may have a unique combination of CDR sequences. Thus, such methods may generate an antibody library having high diversity in VH-CDR1, VH-CDR2, VL-CDR1, VL-CDR2, and VL-CDR3, but little to no diversity in VH-CDR3 (see FIG. 33). Additional selection and screening steps may be performed on the subsequent antibody library to select antibody clones having desired characteristics (FIG. 32, 3205). Finally, an optimal antibody sequence may be determined by computational methods (FIG. 32, 3207). FIG. 34 and FIG. 35 depict methods of screening and selecting for antibody clones with improved characteristics, such as increased thermostability as compared to the initial antibody clone, increased binding affinity for a target epitope as compared to the initial antibody clone, and/or increased species cross-reactivity as compared to the initial antibody clone.

In various aspects, methods are provided for generating antibody libraries. In some cases, the antibody library may comprise a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) a CDR sequence selected from the group consisting of: a VH-CDR1 sequence, a VH-CDR2 sequence, a VH-CDR3 sequence, a VL-CDR1 sequence, a VL-CDR2 sequence, and a VL-CDR3 sequence, wherein the CDR sequence is the same for each antibody of the plurality of antibodies; and (d) a unique combination of remaining CDR sequences selected from the group consisting of: a VH-CDR1 sequence, a VH-CDR2 sequence, a VH-CDR3 sequence, a VL-CDR1 sequence, a VL-CDR2 sequence, and a VL-CDR3. The antibody library can also be referred to herein as a Tumbler Library.

In one example, methods are provided for generating an antibody library comprising a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VH-CDR1 sequence is the same for each antibody of the plurality of antibodies; and (d) the VH-CDR2 sequence, the VH-CDR3 sequence, the VL-CDR1 sequence, the VL-CDR2 sequence, and the VL-CDR3 are present in each antibody of the plurality of antibodies in a different combination. In some cases, the VH-CDR1 sequence is derived from an initial antibody clone.

In another example, methods are provided for generating an antibody library comprising a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VH-CDR2 sequence is the same for each antibody of the plurality of antibodies; and (d) the VH-CDR1 sequence, the VH-CDR3 sequence, the VL-CDR1 sequence, the VL-CDR2 sequence, and the VL-CDR3 are present in each antibody of the plurality of antibodies in a different combination. In some cases, the VH-CDR2 sequence is derived from an initial antibody clone.

In another example, methods are provided for generating an antibody library comprising a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VH-CDR3 sequence is the same for each antibody of the plurality of antibodies; and (d) the VH-CDR1 sequence, the VH-CDR2 sequence, the VL-CDR1 sequence, the VL-CDR2 sequence, and the VL-CDR3 are present in each antibody of the plurality of antibodies in a different combination. In some cases, the VH-CDR3 sequence is derived from an initial antibody clone.

In another example, methods are provided for generating an antibody library comprising a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VL-CDR1 sequence is the same for each antibody of the plurality of antibodies; and (d) the VH-CDR1 sequence, the VH-CDR2 sequence, the VH-CDR3 sequence, the VL-CDR2 sequence, and the VL-CDR3 sequence are present in each antibody of the plurality of antibodies in a different combination. In some cases, the VL-CDR1 sequence is derived from an initial antibody clone.

In another example, methods are provided for generating an antibody library comprising a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VL-CDR2 sequence is the same for each antibody of the plurality of antibodies; and (d) the VH-CDR1 sequence, the VH-CDR2 sequence, the VH-CDR3 sequence, the VL-CDR1 sequence, and the VL-CDR3 sequence are present in each antibody of the plurality of antibodies in a different combination. In some cases, the VL-CDR2 sequence is derived from an initial antibody clone.

In another example, methods are provided for generating an antibody library comprising a plurality of antibodies wherein each antibody of the plurality of antibodies comprises: (a) a VH domain comprising a VH-CDR1 sequence, a VH-CDR2 sequence, and a VH-CDR3 sequence; and (b) a VL domain comprising a VL-CDR1 sequence, a VL-CDR2 sequence, and a VL-CDR3 sequence; wherein (c) the VL-CDR3 sequence is the same for each antibody of the plurality of antibodies; and (d) the VH-CDR1 sequence, the VH-CDR2 sequence, the VH-CDR3 sequence, the VL-CDR1 sequence, and the VL-CDR2 sequence are present in each antibody of the plurality of antibodies in a different combination. In some cases, the VL-CDR3 sequence is derived from an initial antibody clone.

In various aspects, one or more of the VH-CDR1 sequence, the VH-CDR2 sequence, the VH-CDR3 sequence, the VL-CDR1 sequence, the VL-CDR2 sequence, and the VL-CDR3 sequence are naturally occurring. In various aspects, each of the VH-CDR1 sequence, the VH-CDR2 sequence, the VH-CDR3 sequence, the VL-CDR1 sequence, the VL-CDR2 sequence, and the VL-CDR3 sequence are naturally occurring, but are present in each antibody in non-naturally occurring combinations. In various aspects, one or more of the VH-CDR1 sequence, the VH-CDR2 sequence, the VH-CDR3 sequence, the VL-CDR1 sequence, the VL-CDR2 sequence, and the VL-CDR3 sequence are naturally occurring in a human population, or are derived from a human CDR sequence. In various aspects, each of the VH-CDR1 sequence, the VH-CDR2 sequence, the VH-CDR3 sequence, the VL-CDR1 sequence, the VL-CDR2 sequence, and the VL-CDR3 sequence are naturally occurring in a human population, or are derived from a human CDR sequence.

In various aspects, the antibodies of the library can comprise non-naturally occurring combinations of naturally occurring CDRs, such as combinations of CDRs derived from naturally occurring memory B-cells and naïve B-cells, but whose joint appearance on the same antibody would not be naturally occurring. For example, a non-naturally occurring combination of naturally occurring CDRs can comprise at least one CDR derived from a naïve cell while the remaining CDRs can be derived from a memory cell. For example, a non-naturally occurring combination of naturally occurring CDRs can comprise at least one CDR derived from cells of predominantly naïve B-cell origin while the remaining CDRs can be derived from cells of predominantly memory B-cell origin.

The non-naturally occurring combination of naturally occurring CDRs can comprise at least one CDR derived from a naïve cell, while the remaining CDRs are derived from a memory cell. In some cases, at least VL-CDR1 is derived from a naïve cell. In some cases, at least VL-CDR2 is derived from a naïve cell. In some cases, at least VL-CDR3 is derived from a naïve cell. In some cases, at least VH-CDR1 is derived from a naïve cell. In some cases, at least VH-CDR2 is derived from a naïve cell. In some cases, at least VH-CDR3 is derived from a naïve cell.

The non-naturally occurring combination of naturally occurring CDRs can comprise two, three, four, or five CDRs derived from a naïve cell, while the remaining CDRs can be derived from a memory cell. For example, two CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, three CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, four CDRs from CDRs in the group consisting of: VL-CDR1, VL-CDR2, VL-CDR3, VH-CDR1, VH-CDR2, and VH-CDR3 can be derived from a naïve cell while the remaining CDRs can be derived from a memory cell. In another example, five CDRs from CDRs in the group consisting of: VL-CDR1, VL-CDR2, VL-CDR3, VH-CDR1, VH-CDR2, and VH-CDR3 can be derived from a naïve cell while the remaining CDR can be derived from a memory cell.

In another non-limiting example of a non-naturally occurring combination, VL-CDR3 can be derived from a naïve cell, while VH-CDR1, VH-CDR2, VH-CDR3, VL-CDR1, and VL-CDR2 can be derived from a memory cell. In another non-limiting example of a non-naturally occurring combination, VH-CDR3 can be derived from a naïve cell, while VH-CDR1, VH-CDR2, VL-CDR1, VL-CDR2, and VL-CDR3 can be derived from a memory cell. In another non-limiting example of a non-naturally occurring combination, VH-CDR3 and VL-CDR3 can be derived from a naïve cell, while VH-CDR1, VH-CDR2, VL-CDR1, and VL-CDR2 can be derived from a memory cell.

A VH-CDR1 sequence derived from a naïve B-cell can comprise at least 80%, 85%, 90%, 95%, or 99% sequence homology to a naturally occurring VH-CDR1 sequence from a naïve B-cell. A VH-CDR1 sequence derived from a naïve B-cell can be a synthetic VH-CDR1 sequence. A VH-CDR1 sequence derived from a naïve B-cell can comprise 100% sequence homology to a naturally occurring VH-CDR1 sequence from a naïve B-cell. A VH-CDR2 sequence derived from a naïve B-cell can comprise at least 80%, 85%, 90%, 95%, or 99% sequence homology to a naturally occurring VH-CDR2 sequence from a naïve B-cell. A VH-CDR2 sequence derived from a naïve B-cell can be a synthetic VH-CDR2 sequence. A VH-CDR2 sequence derived from a naïve B-cell can comprise 100% sequence homology to a naturally occurring VH-CDR2 sequence from a naïve B-cell. A VH-CDR3 sequence derived from a naïve B-cell can comprise at least 80%, 85%, 90%, 95%, or 99% sequence homology to a naturally occurring VH-CDR3 sequence from a naïve B-cell. A VH-CDR3 sequence derived from a naïve B-cell can be a synthetic VH-CDR3 sequence. A VH-CDR3 sequence derived from a naïve B-cell can comprise 100% sequence homology to a naturally occurring VH-CDR3 sequence from a naïve B-cell.

A VL-CDR1 sequence derived from a naïve B-cell can comprise at least 80%, 85%, 90%, 95%, or 99% sequence homology to a naturally occurring VL-CDR1 sequence from a naïve B-cell. A VL-CDR1 sequence derived from a naïve B-cell can be a synthetic VL-CDR1 sequence. A VL-CDR1 sequence derived from a naïve B-cell can comprise 100% sequence homology to a naturally occurring VL-CDR1 sequence from a naïve B-cell. A VL-CDR2 sequence derived from a naïve B-cell can comprise at least 80%, 85%, 90%, 95%, or 99% sequence homology to a naturally occurring VL-CDR2 sequence from a naïve B-cell. A VL-CDR2 sequence derived from a naïve B-cell can be a synthetic VL-CDR2 sequence. A VL-CDR2 sequence derived from a naïve B-cell can comprise 100% sequence homology to a naturally occurring VL-CDR2 sequence from a naïve B-cell. A VL-CDR3 sequence derived from a naïve B-cell can comprise at least 80%, 85%, 90%, 95%, or 99% sequence homology to a naturally occurring VL-CDR3 sequence from a naïve B-cell. A VL-CDR3 sequence derived from a naïve B-cell can be a synthetic VL-CDR3 sequence. A VL-CDR3 sequence derived from a naïve B-cell can comprise 100% sequence homology to a naturally occurring VL-CDR3 sequence from a naïve B-cell.

The VH-CDR1 sequence, VH-CDR2 sequence, VH-CDR3 sequence, VL-CDR1 sequence, VL-CDR2 sequence, VL-CDR3 sequence, or any combination thereof can be derived from sequence information obtained from a pool of cells of predominantly naïve B-cell origin. The VH-CDR3 sequence, the VL-CDR3 sequence, or the combination thereof can be derived from sequence information obtained from a pool of cells of predominantly naïve B-cell origin. The pool of naïve B-cells can be obtained from a plurality of individuals. The pool of naïve B-cells can comprise less than 0.1%, 1%, 5%, 10%, 20%, or 30% cells not of naïve B-cell origin.

A VH-CDR1 sequence derived from a memory B-cell can comprise at least 80%, 85%, 90%, 95%, or 99% sequence homology to a naturally occurring VH-CDR1 sequence from a memory B-cell. A VH-CDR1 sequence derived from a memory B-cell can be a synthetic VH-CDR1 sequence. A VH-CDR1 sequence derived from a memory B-cell can comprise 100% sequence homology to a naturally occurring VH-CDR1 sequence from a memory B-cell. A VH-CDR2 sequence derived from a memory B-cell can comprise at least 80%, 85%, 90%, 95%, or 99% sequence homology to a naturally occurring VH-CDR2 sequence from a memory B-cell. A VH-CDR2 sequence derived from a memory B-cell can be a synthetic VH-CDR2 sequence. A VH-CDR2 sequence derived from a memory B-cell can comprise 100% sequence homology to a naturally occurring VH-CDR2 sequence from a memory B-cell. A VH-CDR3 sequence derived from a memory B-cell can comprise at least 80%, 85%, 90%, 95%, or 99% sequence homology to a naturally occurring VH-CDR3 sequence from a memory B-cell. A VH-CDR3 sequence derived from a memory B-cell can be a synthetic VH-CDR3 sequence. A VH-CDR3 sequence derived from a memory B-cell can comprise 100% sequence homology to a naturally occurring VH-CDR3 sequence from a memory B-cell.

A VL-CDR1 sequence derived from a memory B-cell can comprise at least 80%, 85%, 90%, 95%, or 99% sequence homology to a naturally occurring VL-CDR1 sequence from a memory B-cell. A VL-CDR1 sequence derived from a memory B-cell can be a synthetic VH-CDR1 sequence. A VL-CDR1 sequence derived from a memory B-cell can comprise 100% sequence homology to a naturally occurring VL-CDR1 sequence from a memory B-cell. A VL-CDR2 sequence derived from a memory B-cell can comprise at least 80%, 85%, 90%, 95%, or 99% sequence homology to a naturally occurring VL-CDR2 sequence from a memory B-cell. A VL-CDR2 sequence derived from a memory B-cell can comprise 100% sequence homology to a naturally occurring VL-CDR2 sequence from a memory B-cell. A VL-CDR3 sequence derived from a memory B-cell can comprise at least 80%, 85%, 90%, 95%, or 99% sequence homology to a naturally occurring VL-CDR3 sequence from a memory B-cell. A VL-CDR3 sequence derived from a memory B-cell can comprise 100% sequence homology to a naturally occurring VL-CDR3 sequence from a memory B-cell.

The VH-CDR1 sequence, VH-CDR2 sequence, VH-CDR3 sequence, VL-CDR1 sequence, VL-CDR2 sequence, VL-CDR3 sequence, or any combination thereof can be derived from sequence information obtained from a pool of cells of predominantly memory B-cell origin. The VH-CDR3 sequence, the VL-CDR3 sequence, or the combination thereof can be derived from sequence information obtained from a pool of cells of predominantly memory B-cell origin. The pool of memory B-cells can be obtained from a plurality of individuals. The pool of memory B-cells can comprise less than 0.1%, 1%, 5%, 10%, 20%, or 30% cells not of memory B-cell origin. The memory B-cells can be CD27+ B-cells. The pool of memory B-cells can comprise less than 0.1%, 1%, 5%, 10%, 20%, or 30% cells not of CD27+ B-cell origin.

The naïve cell can be a naïve B-cell. The naïve B-cell can be a human naïve B-cell. The memory cell can be a memory B-cell. The memory B-cell can be a human memory B-cell. In some instances, a naïve B-cell shows increased diversity of VH-CDR3 and VL-CDR3 sequences compared to VH-CDR3 and VL-CDR3 sequences from a memory B-cell (FIG. 1). The naïve cells and the memory cells can be obtained from a biological sample, such as blood, from an individual or a plurality of individuals. The naïve cells and the memory cells can be physically isolated from this sample using a marker specific to the naïve cells or the memory cells.

A marker can be used to identify, separate, or sort B-cells, naïve B-cells, and memory B-cells from a biological sample. Examples of markers used to identify, separate, or sort a B-cell include, but are not limited to, CD19+. Examples of markers used to identify, separate, or sort a naïve B-cell include, but are not limited to, CD19+, CD27−, IgD+, IgM+, and combinations thereof. Examples of markers used to identify, separate, or sort a memory B-cell include, but are not limited to, CD19+, CD27+, and combinations thereof. In some embodiments, CD27+ is used to sort memory B-cells. Examples of markers used to identify, separate, or sort a class switched memory B-cell include, but are not limited to CD19+, CD27+, CD27+, IgD−, IgM−, and combinations thereof. Examples of markers used to identify, separate, or sort a nonswitched or marginal zone memory B-cell include, but are not limited to, CD19+, CD27+, IgD+, IgM+, and combinations thereof. In some cases, a memory B-cell can be identified, separated, or sorted with the following markers: CD19+, CD27+, IgD−, IgM+, and combinations thereof. The naïve cell from which the VH-CDR3 is derived can be a CD27−/IgM+ B-cell. The memory cell from which the VH-CDR1, VH-CDR2, VL-CDR1, VL-CDR2, and VL-CDR3 are derived can be a CD27+/IgG+ B-cell.

The CDR sequences of the antibody can be CDR sequences found in naïve B-cells and memory B-cells found in an individual or a plurality of individuals. The individual can be a mammal. The mammal can be a human, a non-human primate, mouse, rat, pig, goat, rabbit, horse, cow, cat, or dog. In some cases, the CDR sequences are CDR sequences obtained from a publically available source. Examples of publically available sources of CDR sequences include SAbDab (http://opig.stats.ox.ac.uk/webapps/sabdab-sabpred/Welcome.php) and PylgClassify (http://dunbrack2.fccc.edu/PyIgClassify/).

In various aspects, each antibody in the antibody library may comprise the same scaffold, for example, the same combination of framework sequences (see FIG. 30). In various aspects, the VH domain of each antibody of the antibody library comprises a VH-FR1 sequence, a VH-FR2 sequence, a VH-FR3 sequence, and a VH-FR4 sequence. In some cases, each of the VH-FR1 sequence, the VH-FR2 sequence, the VH-FR3 sequence, and the VH-FR4 sequence is the same for each antibody in the antibody library. In some cases, each of the VH-FR1 sequence, the VH-FR2 sequence, the VH-FR3 sequence, and the VH-FR4 sequence is derived from the same initial antibody clone from which the CDR sequence is derived (see FIG. 30). In some cases, the VH-FR1 sequence may be the same VH-FR1 sequence as the initial antibody clone. In some cases, the VH-FR2 sequence may be the same VH-FR2 sequence as the initial antibody clone. In some cases, the VH-FR3 sequence may be the same VH-FR3 sequence as the initial antibody clone. In some cases, the VH-FR4 sequence may be the same VH-FR4 sequence as the initial antibody clone. In various aspects, the VL domain of each antibody of the antibody library comprises a VL-FR1 sequence, a VL-FR2 sequence, a VL-FR3 sequence, and a VL-FR4 sequence. In some cases, each of the VL-FR1 sequence, the VL-FR2 sequence, the VL-FR3 sequence, and the VL-FR4 sequence is the same for each antibody in the antibody library. In some cases, each of the VL-FR1 sequence, the VL-FR2 sequence, the VL-FR3 sequence, and the VL-FR4 sequence may be derived from the same initial antibody clone from which the CDR sequence is derived (see FIG. 30). In some cases, the VL-FR1 sequence may be the same VL-FR1 sequence as the initial antibody clone. In some cases, the VL-FR2 sequence may be the same VL-FR2 sequence as the initial antibody clone. In some cases, the VL-FR3 sequence may be the same VL-FR3 sequence as the initial antibody clone. In some cases, the VL-FR4 sequence may be the same VL-FR4 sequence as the initial antibody clone.

The framework of the antibody can be a naturally occurring framework. The naturally occurring framework can be a framework found in a mammal. The mammal can be a primate, mouse, rat, pig, goat, rabbit, horse, cow, cat, or dog. The primate can be a human. The framework can comprise at least one variant compared to a naturally occurring framework. The variant can be a mutation, an insertion, or a deletion. The variant can be a variant found in the nucleic acid sequence encoding the antibody or a variant found in the amino acid sequence of the antibody. Any suitable framework sequence can be used, such as those previously used in phase I clinical trials (FIGS. 12A, 12B). As used herein, the framework of an antibody can refer to the framework regions of the variable heavy chain (VH-FR1, VH-FR2, VH-FR3, and VH-FR4), the framework regions of the variable heavy chain (VL-FR1, VL-FR2, VL-FR3, and VL-FR4), or a combination thereof. The framework regions of an antibody in the antibody library can be identical to germline framework regions.

The framework can be a therapeutically optimal framework. The therapeutically optimal framework can comprise at least one, at least two, at least three, at least four, at least five, or all of the following properties selected from the group consisting of: a) previously demonstrated safety in human monoclonal antibodies, b) thermostable; c) not prone to aggregation; d) comprises a single dominant allele at the amino acid level across all human populations; e) comprises different canonical topologies of the CDRs; f) expresses well in bacteria; and g) displays well on a phage. A framework with previously demonstrated safety in human monoclonal antibodies can be a framework of an antibody that has been used in at least a phase I clinical trial. A framework that is thermostable can be a framework that is thermostable at at least 20° C., 30° C., 40° C., 50° C., 60° C., 70° C., 80° C., 90° C., 100° C., or over 100° C. A framework that is thermostable can be a framework that can withstand a temperature increase of at least 3° C. per minute, 4° C. per minute, or 5° C. per minute. A framework that expresses well in bacteria can be a framework that produces a biologically active antibody in the bacteria. The bacteria can be E. coll. The bacteria can be an engineered bacteria. The bacteria can be a bacteria optimized for antibody expression. A framework that displays well on a phage can be a framework that produces a biologically active antibody when displayed on the surface of the phage.

An example of a strategy used to choose a framework is described in FIG. 11, wherein an ideal framework of an antibody can be one that shows structural diversity, has been used successfully in a Phase I clinical trial in humans, has low immunogenicity, shows aggregation resistance, displays fitness, and is thermostable. In some cases, frameworks of antibodies are avoided if they are inherently autoreactive to blood cells (e.g., IGHV4-34), have an inferior stability profile (e.g., IGHV2-5), have a V-gene not found in at least 50% of individuals (e.g., IGHV4-b), show an aggregation prone V-gene (e.g., IGLV6-57), or a combination thereof.

The amino acid sequence of a framework of an antibody herein can comprise more than one dominant allele, wherein there are different dominant alleles in different human populations (FIG. 13 and FIG. 14). For example, the IGHV1-3 framework comprises 3 alleles: IGVH1-3*01, IGVH1-3*02, and IGVH1-3*03, which are found in different frequencies in different human populations (FIG. 26). In some instances, the amino acid sequence of the framework of the antibodies described herein has a single dominant allele in at least two human populations. In some instances, the amino acid sequence of the framework of the antibodies described herein has a single dominant allele in all human populations. A framework with one dominant allele can be a framework in which one allele is found in at least 50%, at least 75%, or at least 90% in at least two human populations. A framework with one dominant allele can be a framework in which one allele is found in at least 50%, at least 75%, or at least 90% in at least twelve human populations. In some cases, the framework regions of the VH domain are framework regions from IGHJ4, IGHV1-46, IGHV1-69, IGHV3-15, or IGHV3-23. In some cases, the framework regions of the VH domain are framework regions from IGHV2-5, IGHV3-7, IGVH4-34, IGHV5-51, IGHV1-24, IGHV2-26, IGHV3-72, IGHV3-74, IGHV3-9, IGHV3-30, IGHV3-33, IGHV3-53, IGHV3-66, IGHV4-30-4, IGHV4-31, IGHV4-59, IGHV4-61, or IGHV5-51. In some cases, the framework regions of the VH domain of the antibodies in the antibody library are framework regions from IGHV1-46, IGHV3-23, or a combination thereof. In some cases, the frameworks regions of the VL domain of the antibodies in the antibody library are framework regions from IGKV1-39, IGKV2-28, IGKV3-15, IGKV4-1, IGKV1-5, IGKV1-12, IGKV1-13, IGKV3-11, IGKV3-20, or a combination thereof. In one example, a subset of antibodies in the antibody library can have framework regions of the VH domain from IGHV1-46 and framework regions of the VL domain from IGKV1-39, while the remaining antibodies in the antibody library have framework regions of the VH domain from IGHV1-46 and framework regions of the VL domain from IGKV2-28.

Disclosed herein, in some cases, are nucleic acid sequences encoding an antibody described herein. The nucleic acid sequence can be a DNA or an RNA sequence. The nucleic acid can be inserted into a vector. The vector can be a phage. The phage can be a phagemid or a bacteriophage. The phagemid can be pMID21. The bacteriophage can be DY3F63, an M13 phage, fd filamentous phage, T4 phage, T7 phage or λ phage. In some cases a phagemid can be introduced into a microbe in combination with a bacteriophage (i.e. a ‘helper’ phage). The microbe can be a filamentous bacteria. The filamentous bacteria can be Escherichia coli.

The antibody libraries described herein comprise a plurality of antibodies. The plurality of antibodies can be at least 1.0×10⁶, 1.0×10⁷, 1.0×10⁸, 1.0×10⁹, 1.0×10¹⁰, 2.0×10¹⁰, 3.0×10¹⁰, 4.0×10¹⁰, 5.0×10¹⁰, 6.0×10¹⁰, 7.0×10¹⁰, 8.0×10¹⁰, 9.0×10¹⁰, or 10.0×10¹⁰ antibodies. The plurality of antibodies can be at least 1.0×10¹¹ antibodies. The plurality of antibodies can be at least 7.6×10¹⁰ antibodies. Such libraries can be unique because of their high diversity. For example, in any of the libraries herein at least 2%, at least 5%, at least 10%, at least 15%, at least 20%, at least 25%, at least 30%, or at least 35% of the plurality of antibodies can be unique. In some instances, a library has more than 7.0×10¹⁰ antibodies of which at least 20% of the plurality of antibodies are unique. The unique antibody can vary by at least one nucleic acid or at least one amino acid residue relative to the other antibodies of the antibody library.

In some instances, the antibody library comprises at least 1.0×10⁵ antibodies of which at least 80% of the plurality of antibodies are functional. In some instances, the antibody library comprises at least 7.0×10¹⁰ antibodies of which at least 80% of the plurality of antibodies are functional. In some instances, the antibody library comprises at least 7.6×10¹⁰ antibodies of which at least 80% of the plurality of antibodies are functional.

In some instances, the antibody library comprises at least 1.0×10⁵ antibodies of which at least 85% of the plurality of antibodies are functional. In some instances, the antibody library comprises at least 7.0×10¹⁰ antibodies of which at least 85% of the plurality of antibodies are functional. In some instances, the antibody library comprises at least 7.6×10¹⁰ antibodies of which at least 85% of the plurality of antibodies are functional.

In some instances, the antibody library comprises at least 1.0×10⁵ antibodies of which at least 90% of the plurality of antibodies are functional. In some instances, the antibody library comprises at least 7.0×10¹⁰ antibodies of which at least 90% of the plurality of antibodies are functional. In some instances, the antibody library comprises at least 7.6×10¹⁰ antibodies of which at least 90% of the plurality of antibodies are functional.

In some instances, the antibody library comprises at least 1.0×10⁵ antibodies of which at least 95% of the plurality of antibodies are functional. In some instances, the antibody library comprises at least 7.0×10¹⁰ antibodies of which at least 95% of the plurality of antibodies are functional. In some instances, the antibody library comprises at least 7.6×10¹⁰ antibodies of which at least 95% of the plurality of antibodies are functional.

In some instances, the antibody library comprises at least 1.0×10⁵ antibodies of which at least 99% of the plurality of antibodies are functional. In some instances, the antibody library comprises at least 7.0×10¹⁰ antibodies of which at least 99% of the plurality of antibodies are functional. In some instances, the antibody library comprises at least 7.6×10¹⁰ antibodies of which at least 99% of the plurality of antibodies are functional.

In various aspects, the methods provided herein may generate antibody libraries having high diversity in one or more CDR sequences. In some cases, the antibody library may have high diversity in a VH-CDR1 sequence. In some cases, the antibody library may have high diversity in a VH-CDR2 sequence. In some cases, the antibody library may have high diversity in a VH-CDR3 sequence. In some cases, the antibody library may have high diversity in a VL-CDR1 sequence. In some cases, the antibody library may have high diversity in a VL-CDR2 sequence. In some cases, the antibody library may have high diversity in a VL-CDR3 sequence. In some cases, the antibody library may have high diversity in a CDR sequence not derived from an initial antibody clone, and low or no diversity in the CDR sequence derived from the initial antibody clone. In some cases, an antibody library with high diversity may comprise at least 1×10³, 5×10³, 1×10⁴, 5×10⁴, 1×10⁵, 5×10⁵, 1×10⁶, 5×10⁶, or more unique sequences in a particular CDR. In some cases, an antibody library may comprise high diversity in five of the six CDR sequences, for example, an antibody library may comprise high diversity in five CDR sequences selected from the group consisting of: a VH-CDR1 sequence, a VH-CDR2 sequence, a VH-CDR3 sequence, a VL-CDR1 sequence, a VL-CDR2 sequence, and a VL-CDR3 sequence. In such cases, the remaining CDR sequence may have low or no diversity. In some cases, the remaining CDR sequence is the same CDR sequence for each antibody. In some cases, the remaining CDR sequence is derived from an initial antibody clone.

In various aspects, the methods provided herein may generate at least one antibody having an improvement in at least one characteristic as compared to an initial antibody clone. In some cases, at least one antibody of the antibody library may exhibit an improvement in thermal stability as compared to an initial antibody clone. For example, at least one antibody of the antibody library may exhibit at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, or greater than 100× thermal stability as compared to an initial antibody clone. In some cases, at least one antibody of the antibody library may exhibit greater affinity for a target epitope as compared to an initial antibody clone. For example, at least one antibody of the antibody library may exhibit at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, or greater than 100× affinity for a target epitope as compared to an initial antibody clone. In some cases, at least one antibody of the antibody library may exhibit an improvement in species selectivity. For example, at least one antibody of the antibody library may exhibit at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, or greater than 100× affinity for a target epitope of a specific species as compared to an initial antibody clone. In some cases, at least one antibody of the antibody library may exhibit increased species cross-reactivity as compared to an initial antibody clone. For example, at least one antibody of the antibody library may have high affinity for epitope A from species A (e.g., cynomolgus monkey), and may have high affinity for epitope A from species B (e.g., human). In some cases, at least one antibody of the antibody library may exhibit at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, or greater than 100× affinity for epitope A from species A as compared to an initial antibody clone, and at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, or greater than 100× affinity for epitope A from species B as compared to an initial antibody clone. FIGS. 31A & B depict a non-limiting example of selecting for an antibody clone exhibiting improved thermal stability, improved affinity for epitope A from cynomolgus monkey, and improved affinity for epitope A from human.

In various aspects, the methods described herein may generate an antibody having thermal stability. In some cases, an antibody of the antibody library may be thermostable at temperatures from about 50° C. to about 90° C. For example, an antibody of the antibody library may be thermostable at temperatures of at least 50° C., 55° C., 60° C., 65° C., 70° C., 75° C., 80° C., 85° C., or 90° C.

In various aspects, the methods described herein may generate an antibody having high affinity for a target epitope. For example, an antibody of the antibody library may bind to a target epitope with a dissociation constant (K_{d}) of less than about 50 nM, 25 nM, 10 nM, 5 nM, 1 nM, 900 pM, 800 pM, 700 pM, 600 pM, 500 pM, 400 pM, 300 pM, 200 pM, 100 pM, 50 pM, 25 pM, 10 pM, 5 pM, 1 pM, 900 fM, 800 fM, 700 fM, 600 fM, 500 fM, 400 fM, 300 fM, 200 fM, 100 fM, 50 fM, 25 fM, 10 fM, 5 fM, 1 fM, or less.

### Certain Terminology

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with a disorder, as well as those prone to have the disorder, or those in whom the disorder is to be prevented.

The term “antibody” as used herein refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and portions thereof; including, for example, an immunoglobulin molecule, a polyclonal antibody, a monoclonal antibody, a recombinant antibody, a chimeric antibody, a humanized antibody, a CDR-grafted antibody, F(ab)₂, Fv, scFv, IgGΔCH₂, F(ab′)2, scFv2CH₃, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM) a modified antibody, and a synthetic antibody (including, without limitation non-depleting IgG antibodies, T-bodies, or other Fc or Fab variants of antibodies).

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### EXAMPLES

### Example 1: Generation of a SuperHuman Library (SHL) 2.0

A superhuman library was generated using the following steps:
1. The best 4 VH and best 4 VK frameworks from a human repertoire of 3500 combinations (IGHV1-46, IGHV1-69, IGHV3-15, IGHV3-23 for heavy and IGKV1-39, IGKV2-28, IGKV3-15, IGKV4-1 for light) were selected based on a combination of 1) previous demonstrated safety in human mAbs, 2) thermostability, 3) not aggregation prone, 4) a single dominant allele in the frameworks at the amino acid level across all human populations (i.e. not a racist medicine), 5) different canonical topologies of the CDRs, 6) express well in bacteria and display well on phage.
2. Blood was obtained from 140 subjects.
3. Naïve (CD27−/IgM+) cells and memory (CD27+/IgG+) cells were sorted from the blood.
4. Pools were checked for quality using next generation sequencing (NGS), and pools with problematic diversity or biochemical liabilities were rejected.
5. VH-CDR3 sequences from naïve cells were PCR amplified with universal primers.
6. VH-CDR1, VH-CDR2, VL-CDR1, VL-CDR2, and VL-CDR3 sequences from memory cells were PCR amplified with framework specific primers.
7. Order frameworks as synthetically produced germline segments.
8. Nucleic acid libraries were assembled using PCR-OE.
9. The assemblies from step 8 were checked for quality using NGS sequencing.
10. Light chains were cloned into a vector with a stuffer VH.
11. In-frame material was selected by protein A or protein L after thermal pressure.
12. Heavy chains were cloned into the vector to replace the stuffer VH.
13. Microbes were transformed with the vectors generated at the end of step 12 using electroporation.

### Example 2: Screening for Affinity to PD1

A primary screen of two 96-well plates of clones randomly selected after round 4 SuperHuman panning of PD1.

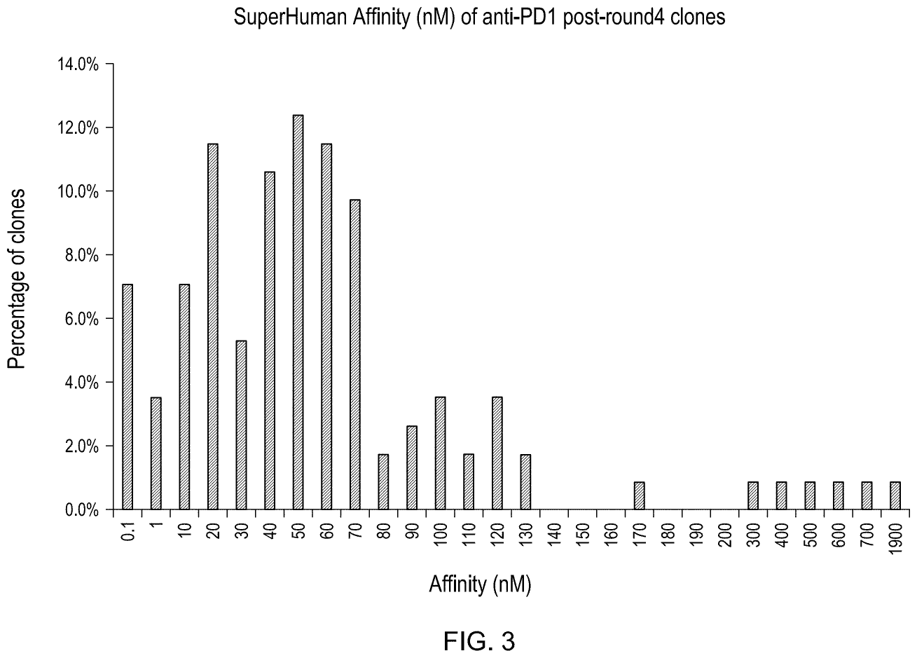

Samples were immediately assayed on Carterra high-throughput kinetics instrument, bypassing ELISA screening (FIG. 3). A majority of the hits were positive and of 184 sequenced, 98 were unique.

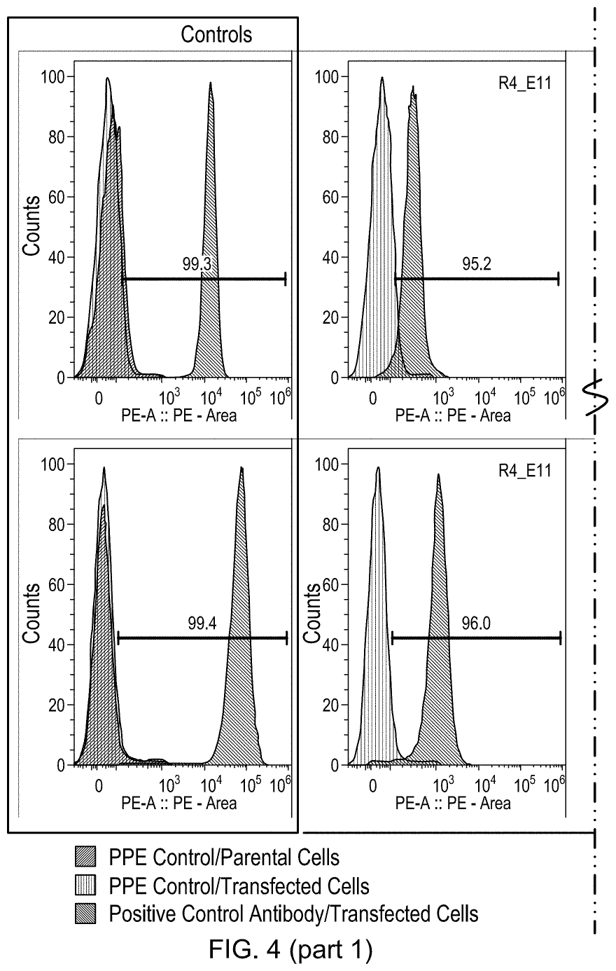

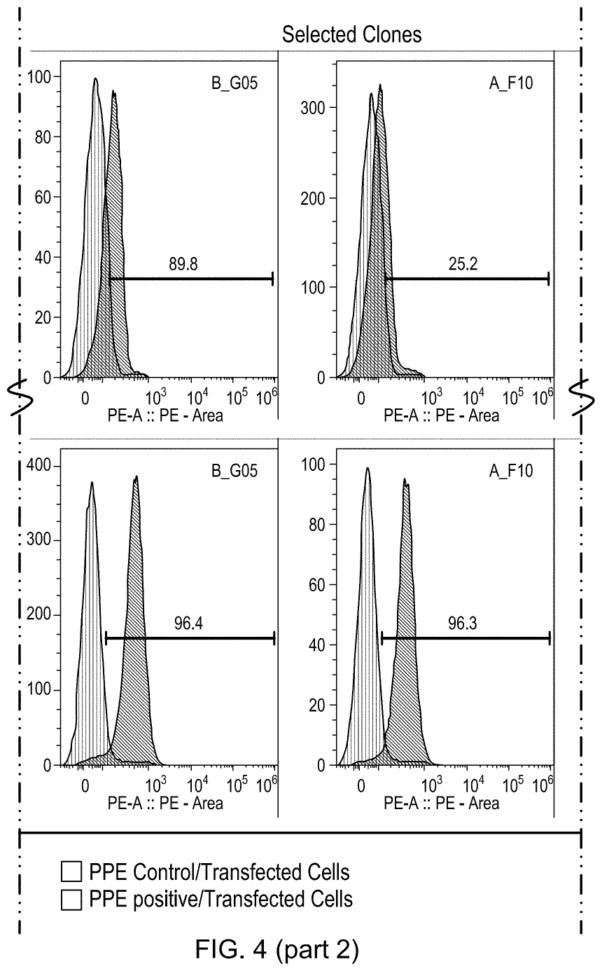

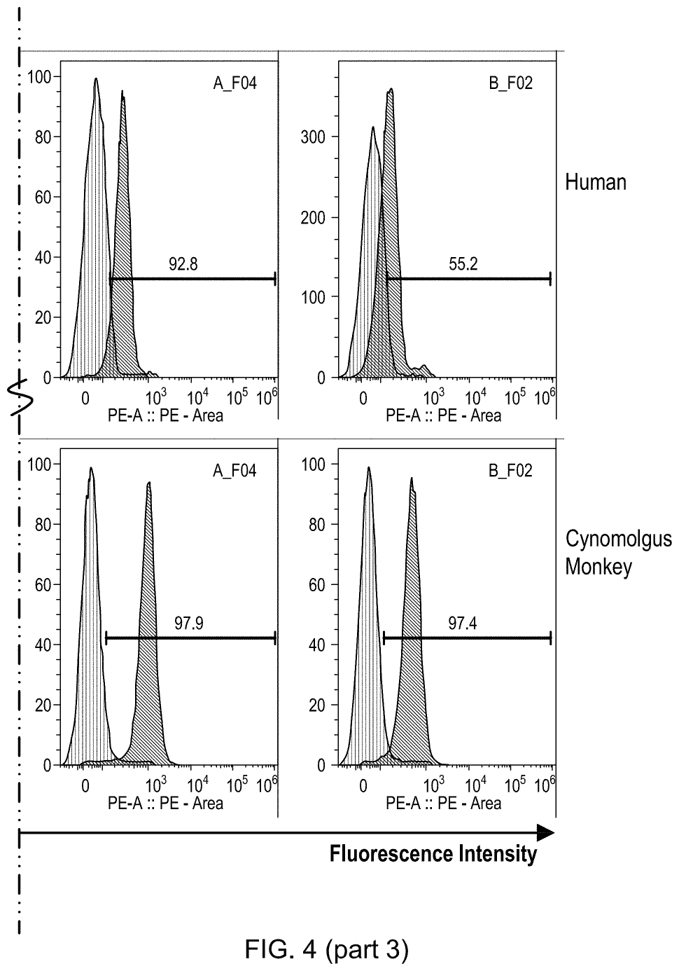

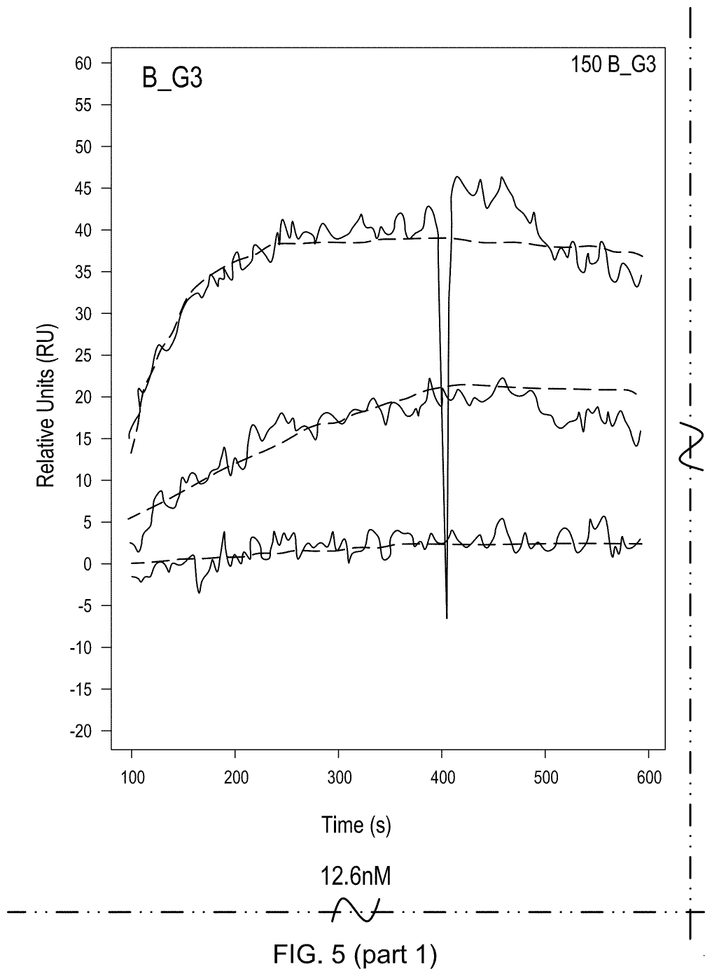

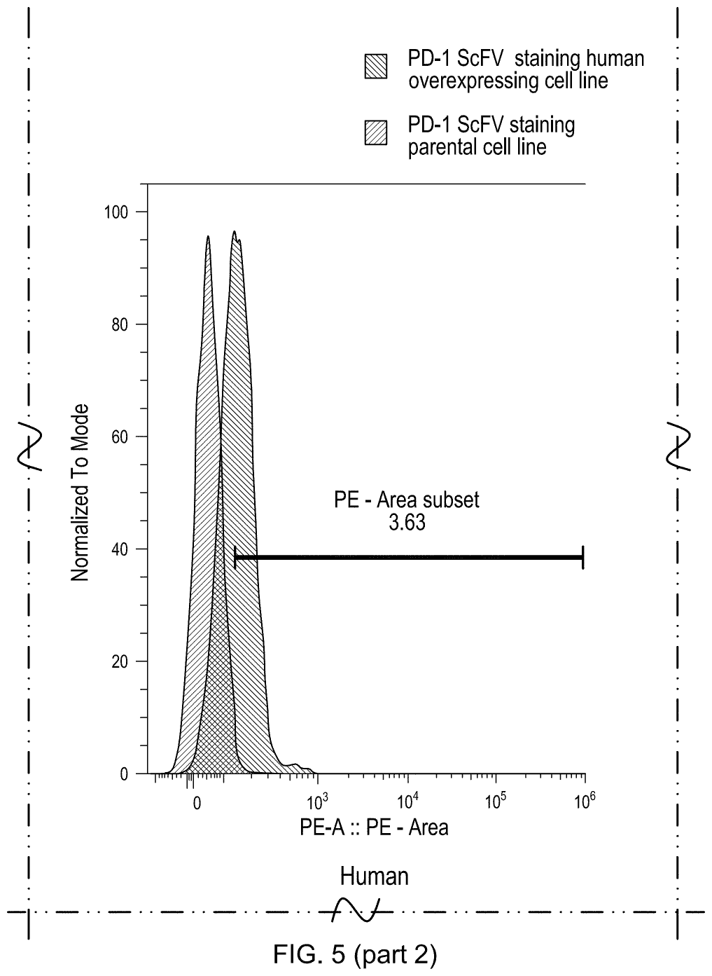

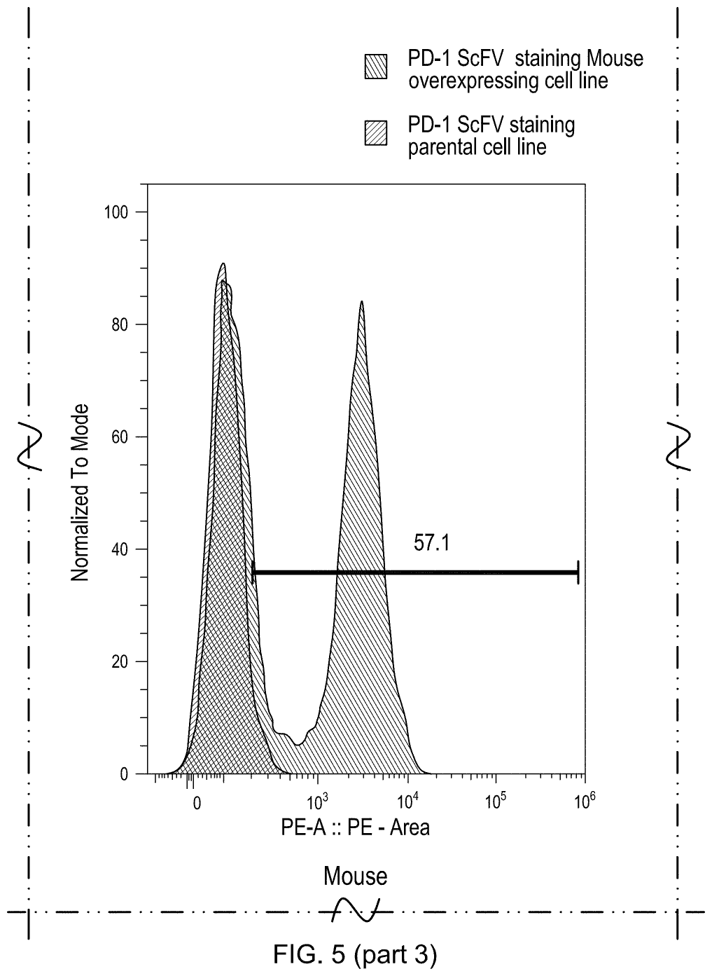

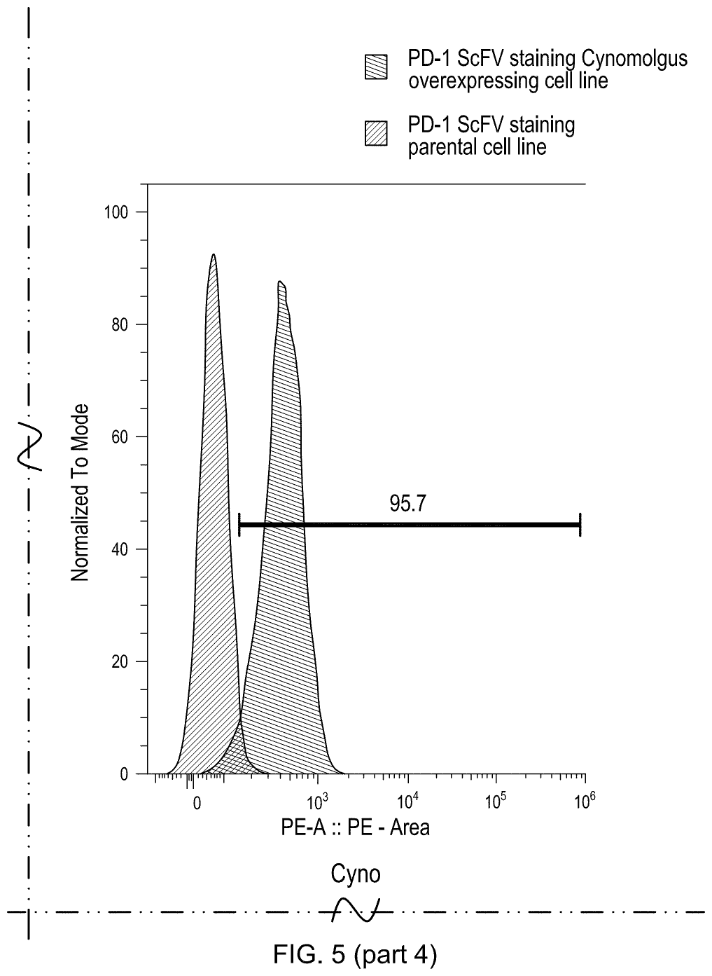

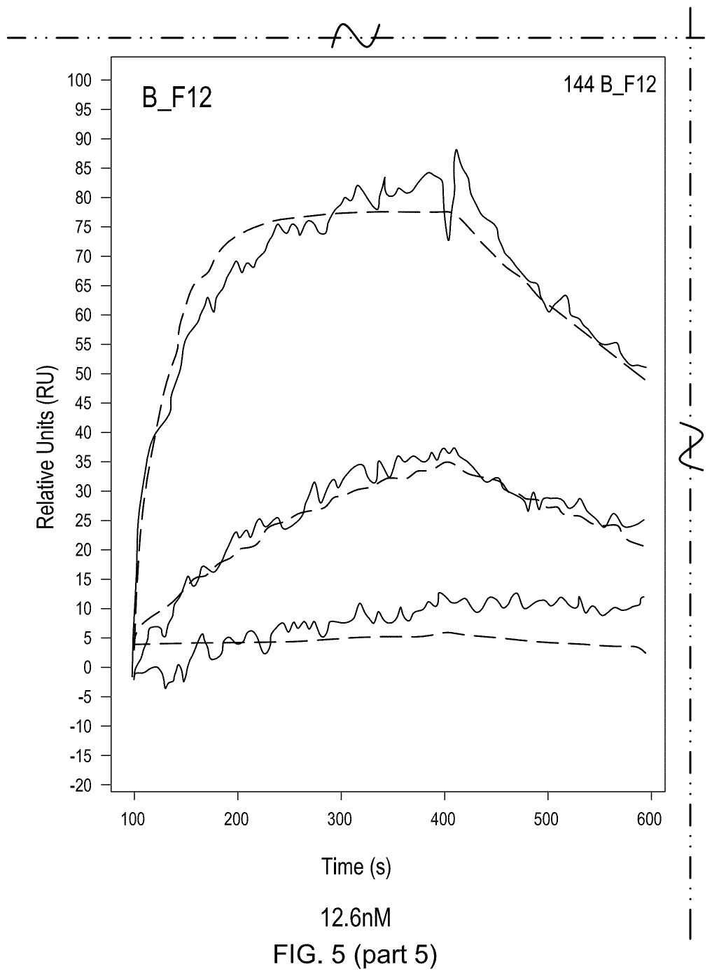

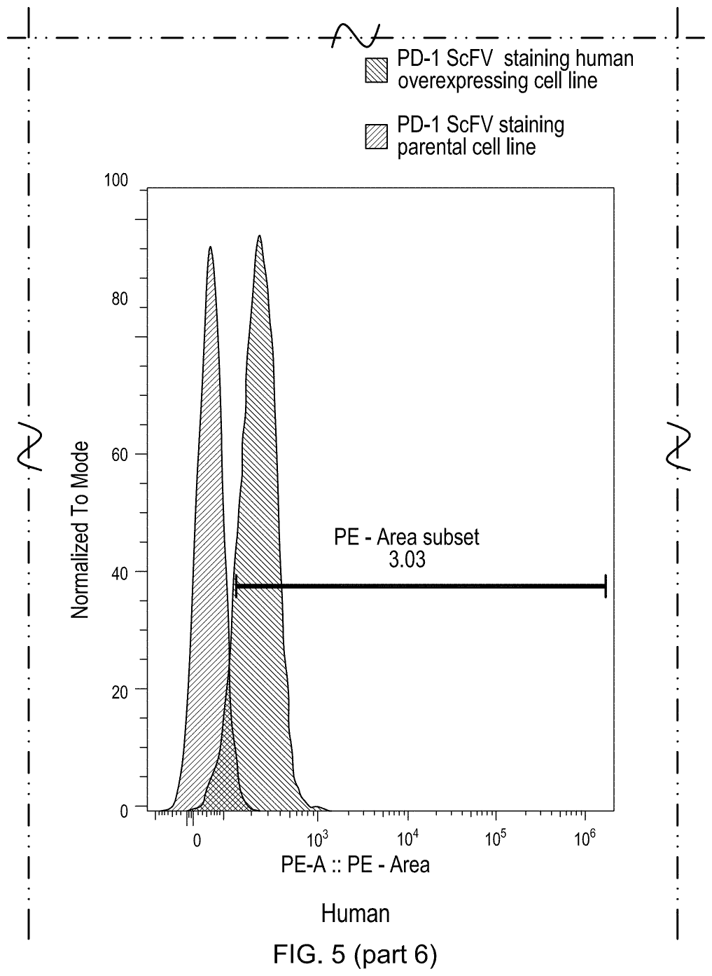

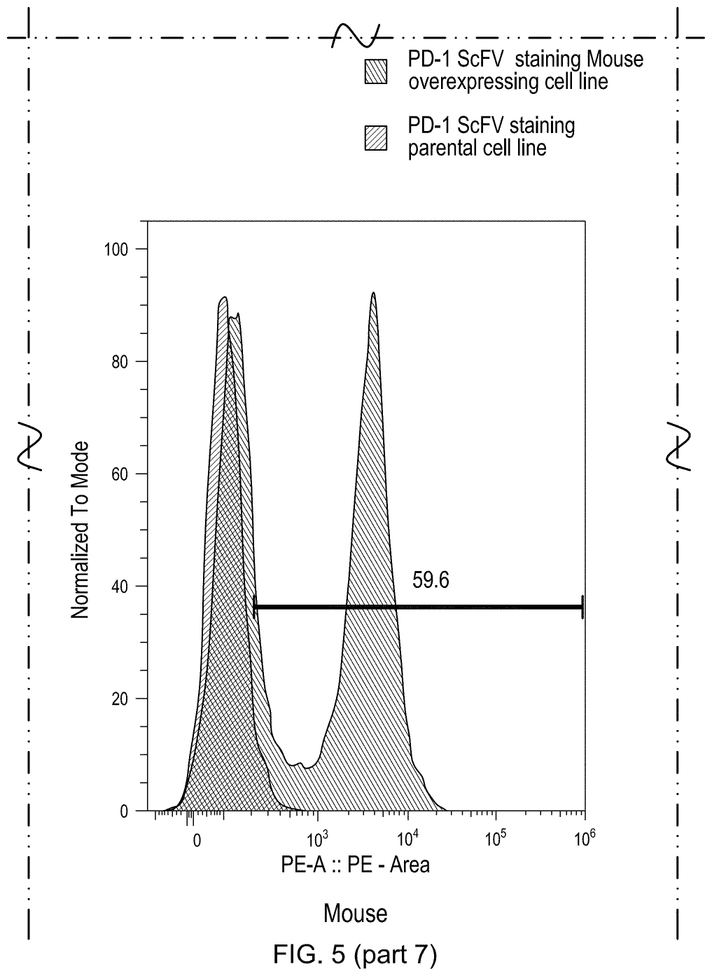

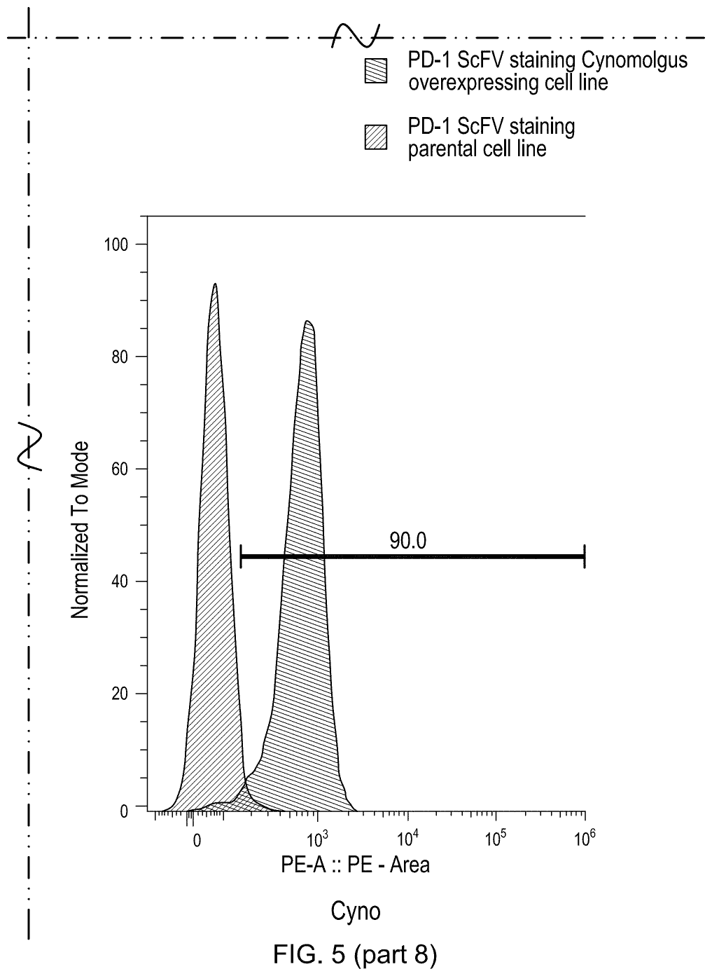

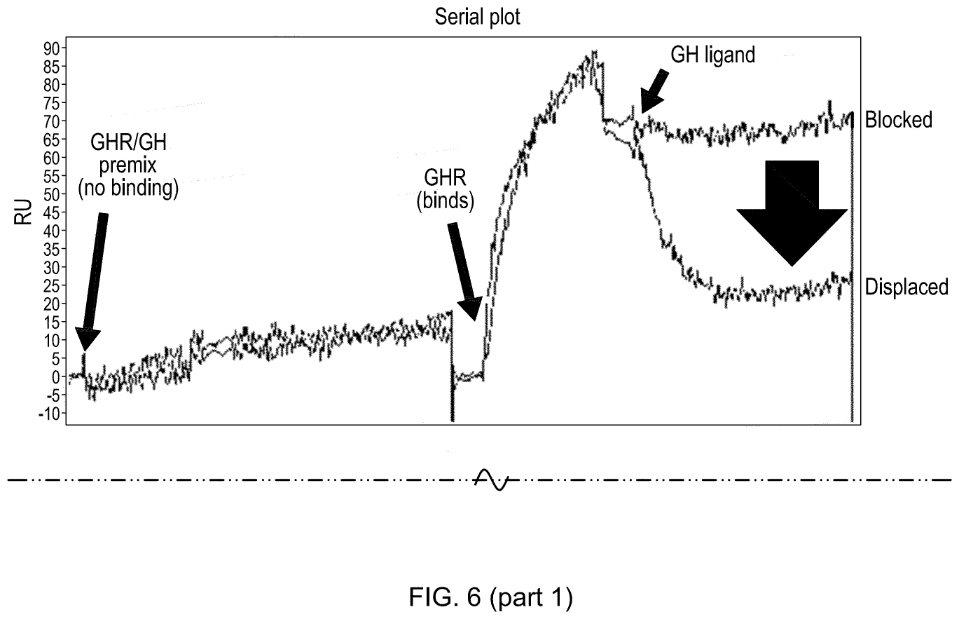

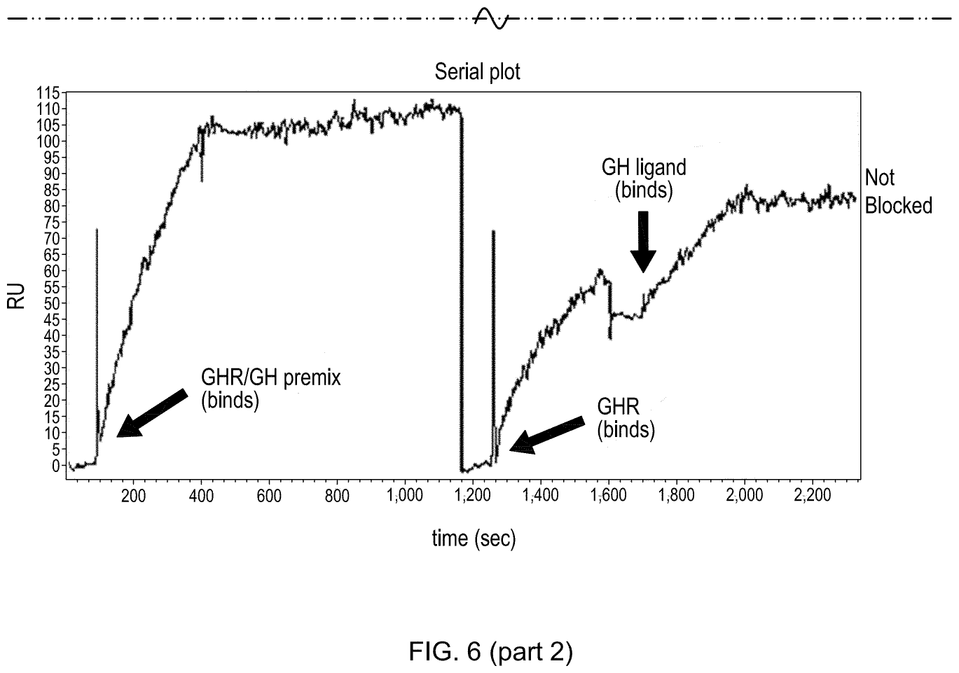

Clones showing affinity to PD1 were confirmed against human and cynomolgus monkey PD1 (FIG. 4), (FIG. 5).

### Example 3: bGal ELISA and Sanger Screening of 2 Plates of Antibody Clones

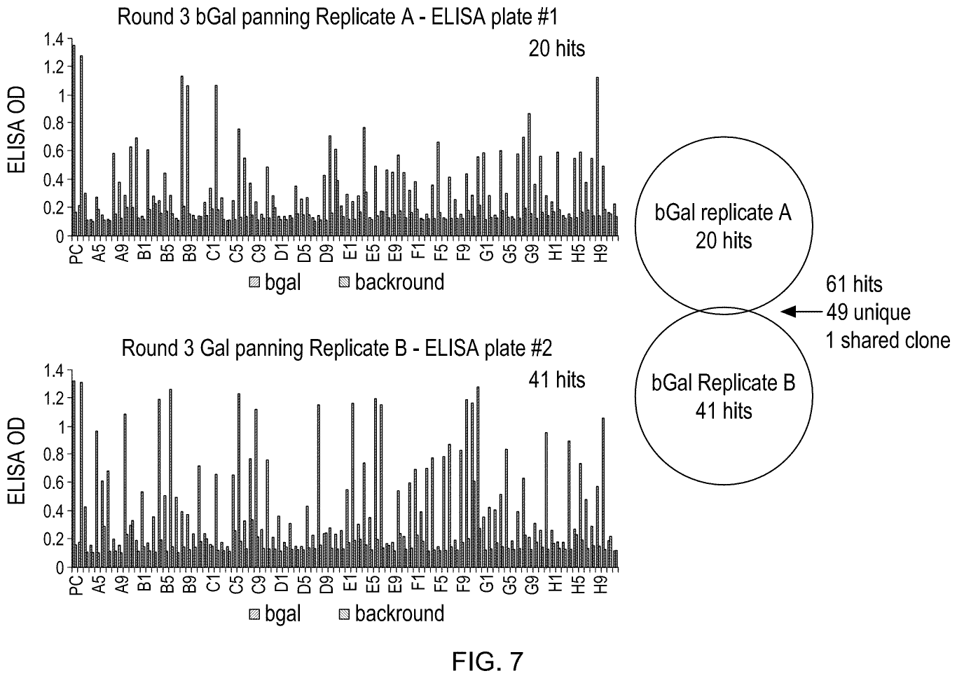

An ELISA panning antibody clones from two plates against bGal was carried out (FIG. 7).

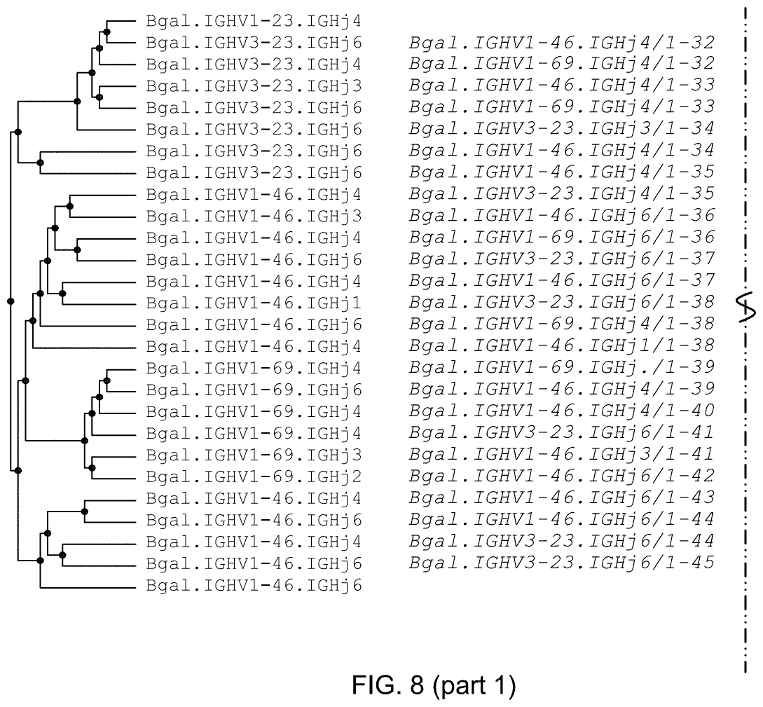

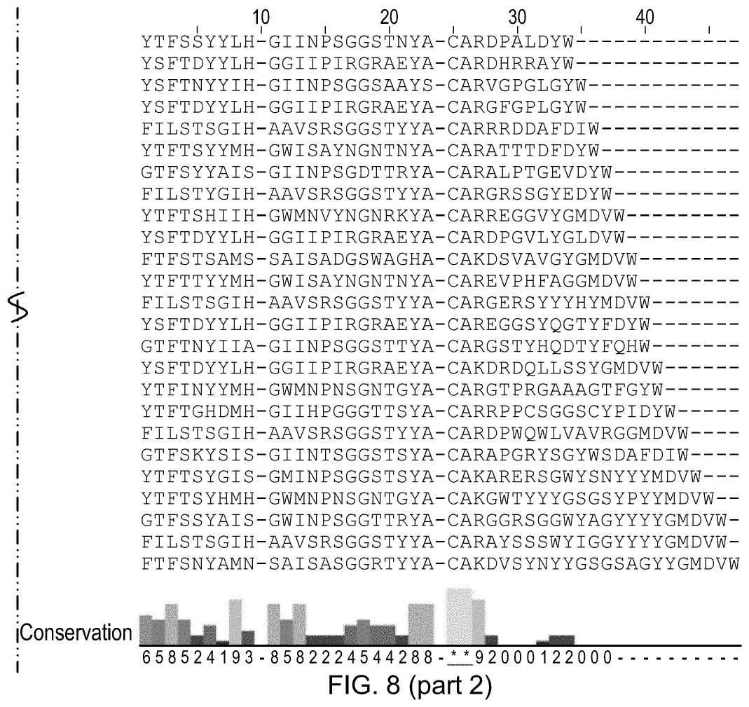

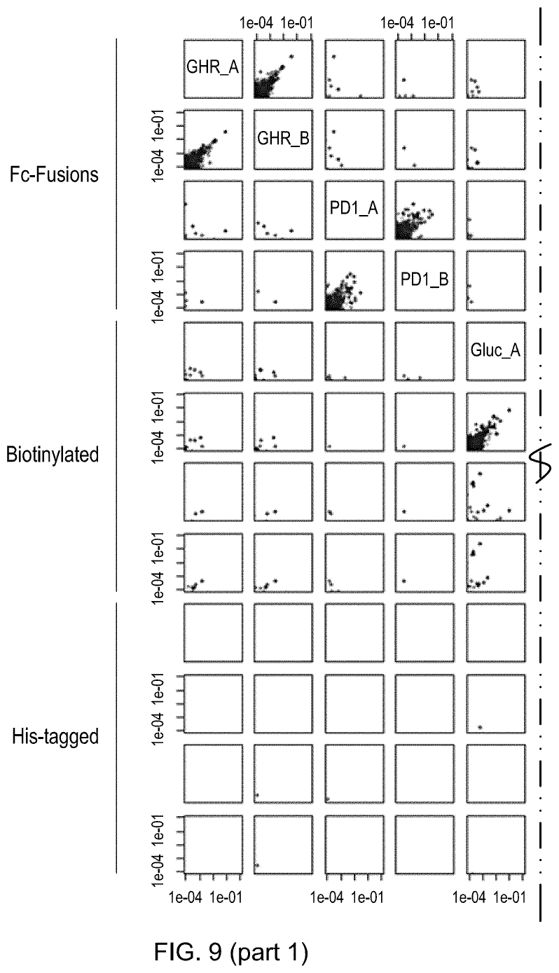

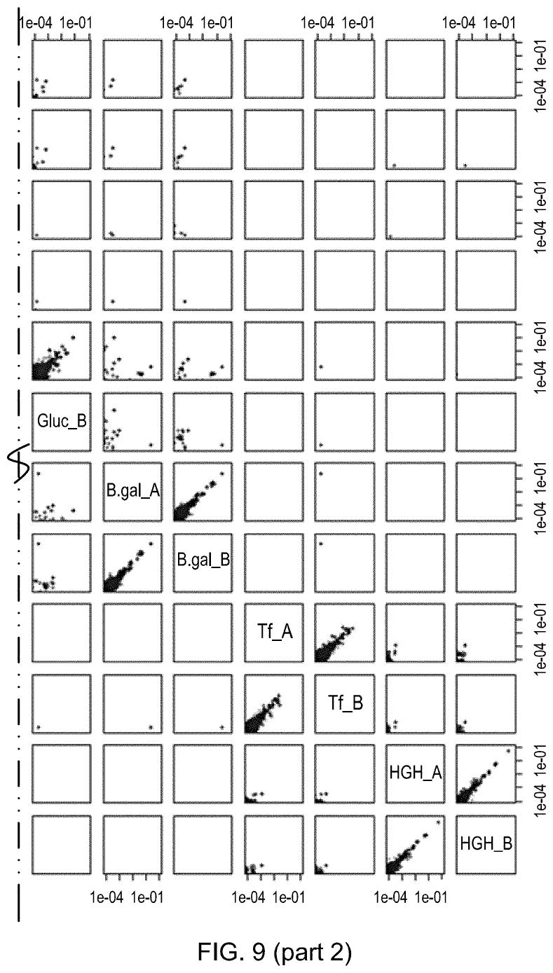

Sanger sequencing of these clones was also carried out (FIG. 8). Extreme diversity of round 3 outputs ensured that hits against any epitope can be recovered by screening a few 96-well plates of clones.

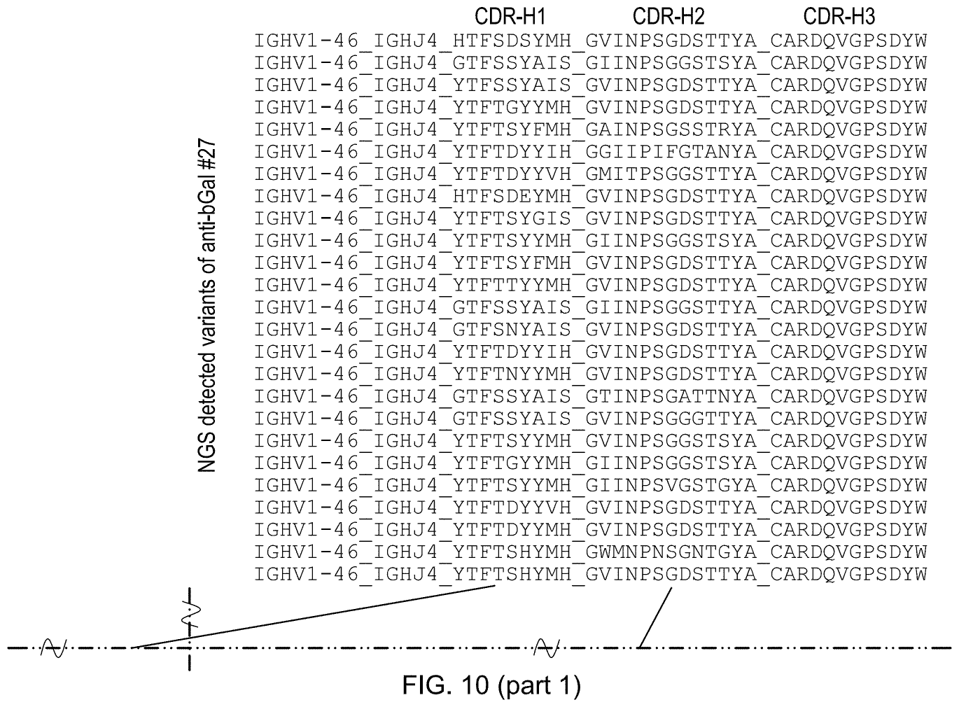

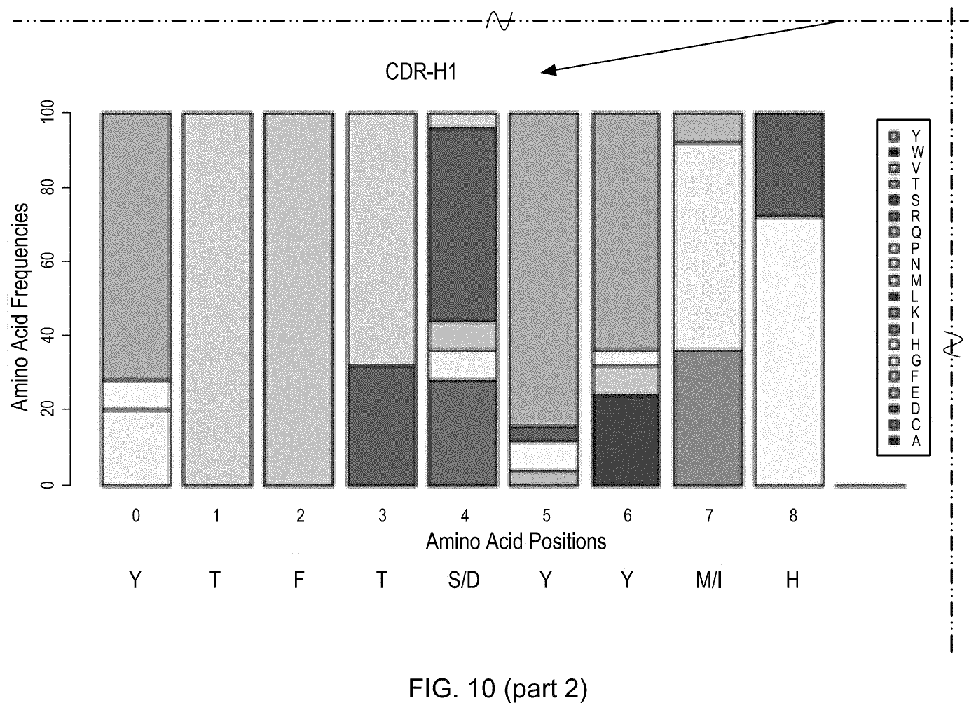

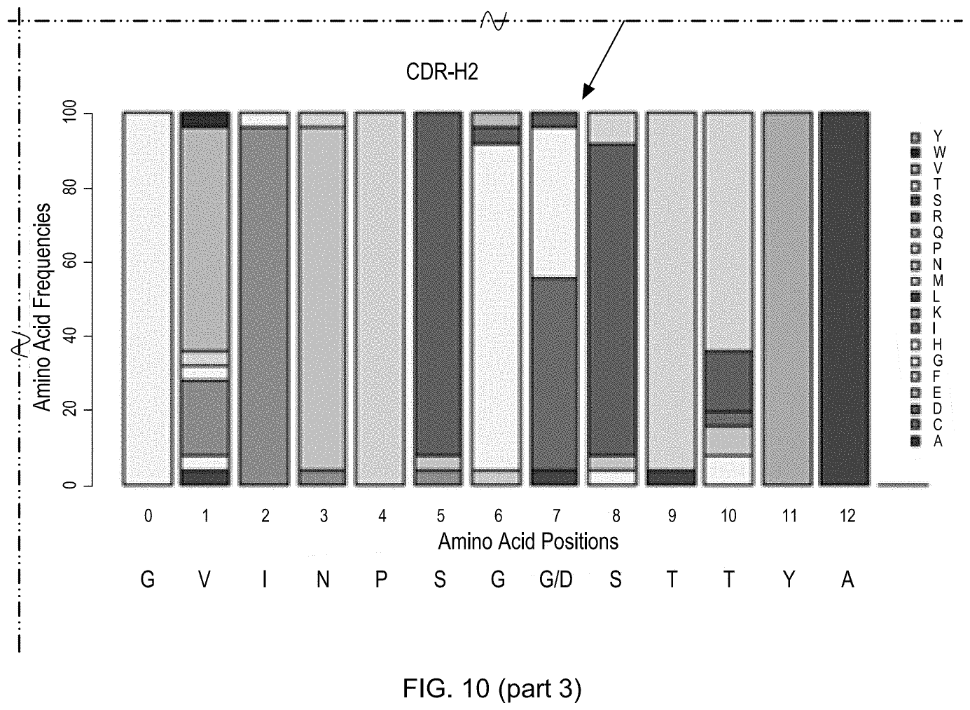

Diversity was found not only in the VH-CDR3 (CDR-H3) sequences, but also in the VH-CDR1 (CDR-H1) and VH-CDR2 (CDR-H2) sequences (FIG. 10).

### Example 4: Combining Design and Selection Processes to Produce an Antibody Library with Diverse VH and VK Sequences

Functional selection for expression and thermostability during construction was applied to produce a library with over 95% functional diversity across 40 million light chains. The antibody library was created with 7.6×10¹⁰ transformants.

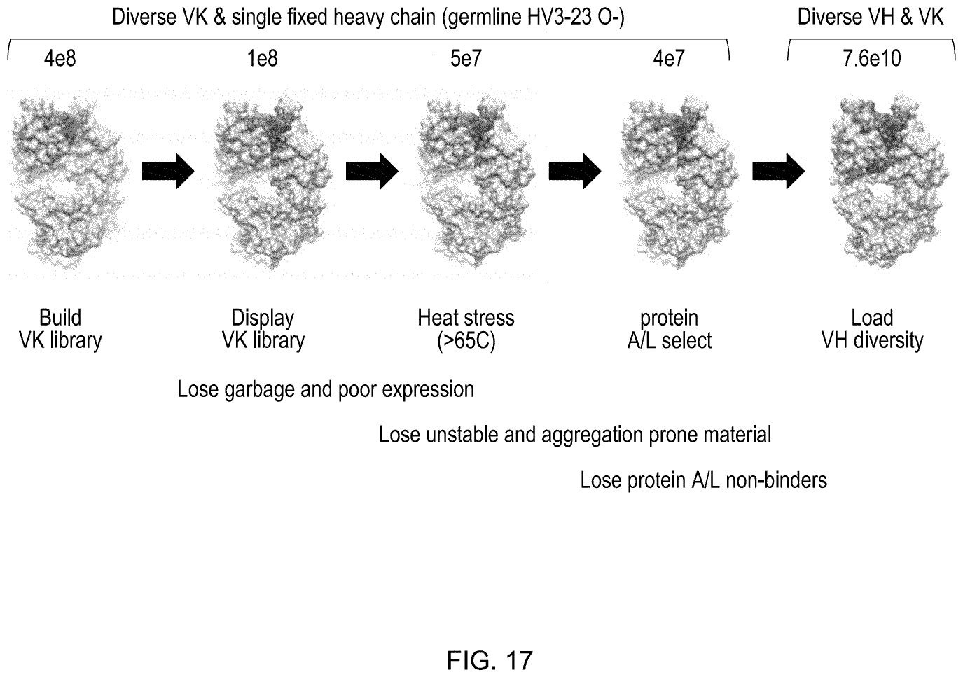

First, a VK (kappa light chain) library was produced by cloning into a vector the desired light chain and temporary stuffer VH sequence. The VK library was displayed and subjected to a heat stress at over 65° C. In-frame material was selected using protein A/L. The stuffer VH sequence in the library resulting from the protein A/L selection was replaced with the target VH sequence (FIG. 17).

### Example 5: Generation of a SuperHuman Library (SHL) 3.0

A superhuman library is generated using the following steps:
1. Six antibody frameworks (IGHV1-46, IGHV3-23, IGKV1-39, IGKV2-28, IGKV3-15, and IGKV4-1) are selected based on a combination of 1) previous demonstrated safety in human mAbs, 2) thermostability, 3) not aggregation prone, 4) a single dominant allele in the frameworks at the amino acid level across all human populations (i.e. not a racist medicine), 5) different canonical topologies of the CDRs, 6) express well in bacteria and display well on phage.
2. Blood is obtained from 50-100 subjects.
3. Naïve (CD27−/IgM+ or CD27−/IgD+) cells and memory cells are sorted from the blood.
4. Pools are checked for quality using next generation sequencing (NGS), and pools with problematic diversity or biochemical liabilities are rejected.
5. VH-CDR3 sequences from naïve cells are PCR amplified with universal primers.
6. Favorable VH-CDR1, VH-CDR2, VL-CDR1, VL-CDR2, and VL-CDR3 sequences without liabilities are selected by DNA synthesis based on (1) being observed present in human natural antibodies, (2) being observed not under-performing under selection of SuperHuman 2.0 against a variety of antigens, (3) being free of biochemical liabilities (C, exposed M, deamination sites, acid hydrolysis sites, N-linked glycosylation sites, amber stop codons, opal stop codons, highly positively charged), (4) not being more mutated than a threshold (e.g., no more than 3 amino acid mutations per CDR). Stated differently, VH-CDR1, VHCDR2, VL-CDR1, VL-CDR2, and VL-CDR3 sequences are synthesized if they meet the following criteria:
   a) have no more than 4 amino acid mutations away from the respective germline CDR for the respective framework used; and
   b) are identified as present in at least 2 of the subjects during NGS and are enriched without a fitness disadvantage when evaluating a pool of 55,000 hits against 11 antigens from SuperHuman 2.0 (Example 1), or have not been observed in a person but to have heavily enriched during panning in the same SuperHuman 2.0 pool; and
   c) do not contain any biochemical liabilities (N-linked glycosylation, deamination, acid hydrolysis, positive charge endopeptidic cleavage, free cysteines, free methionines, alternative stop codons, cryptic splice sites, tev cleagage sites, or overly positively charged CDRs).
7. Order frameworks as synthetically produced 100% germline segments with no mutations.
8. Nucleic acid libraries are assembled using PCR-OE or another method for DNA assembly.
9. The assemblies from step 8 are checked for quality using NGS sequencing.
10. Light chains are cloned into a vector with a stuffer VH
11. In-frame material is selected by protein A or protein L after thermal pressure.
12. Heavy chains are cloned into the vector to replace the stuffer VH.
13. Microbes are transformed with the vectors generated at the end of step 12 using electroporation.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An antibody library that comprises a plurality of antibodies,
wherein each antibody of the plurality of antibodies comprises:
a) a VH domain that comprises a VH-CDR1 sequence, a VH-CDR2 sequence, a VH-CDR3 sequence; and
b) a VL domain that comprises a VL-CDR1 sequence, a VL-CDR2 sequence, a VL-CDR3 sequence; and
wherein:
a) at least one of the VH-CDR3 sequence and the VL-CDR3 sequence is derived from a naïve B-cell;
b) if only one of the VH-CDR3 sequence and the VL-CDR3 sequence is derived from the naïve B-cell, then the VH-CDR3 sequence or VL-CDR3 sequence not derived from the naïve B-cell is derived from a memory cell; and
c) the VH-CDR1 sequence, VH-CDR2 sequence, VL-CDR1 sequence, and VL-CDR2 sequence are derived from a memory B-cell.

2. The antibody library of claim 1, wherein the at least one of the VH-CDR3 sequence and the VL-CDR3 sequence derived from a naïve B-cell is a naturally occurring sequence.

3. The antibody library of claim 1, wherein the VH-CDR3 sequence or VL-CDR3 sequence derived from a memory cell is a naturally occurring sequence.

4. The antibody library of claim 1, wherein the VH-CDR1 sequence, VH-CDR2 sequence, VL-CDR1 sequence, and VL-CDR2 sequence derived from a memory B cell are naturally occurring sequences.

5. (canceled)

6. (canceled)

7. (canceled)

8. The antibody library of claim 1, wherein the VL domain is a Vκ domain or a Vλ domain.

9. The antibody library of claim 1, wherein the naïve B-cell is a CD27−/IgM+ B-cell or a CD27−/IgD+ B-cell.

10. The antibody library of claim 1, wherein the memory B-cell is selected from the group consisting of: a CD27+/IgG+ B-cell, a CD27+/IgM+ B-cell, an IgA+ B-cell, and a combination thereof.

11. The antibody library of claim 1, wherein the naïve B-cell and memory B-cell are from a sample comprising a plurality of naïve B-cells and memory B-cells sampled from a plurality of individuals.

12. The antibody library of claim 11, wherein the plurality of individuals is at least 50 individuals.

13. The antibody library of claim 1, wherein the plurality of antibodies are expressed on a surface of a plurality of phages.

14. The antibody library of claim 13, wherein the plurality of phages comprise bacteriophages or phagemids.

15. The antibody library of claim 13, wherein each phage of the plurality of phages comprises a nucleic acid sequence that encodes: i) an antibody of the plurality of antibodies, and ii) a gene encoding a phage coat protein.

16. The antibody library of claim 15, wherein the phage coat protein is a protein gIII.

17. The antibody library of claim 15, wherein expression of the nucleic acid sequence of each phage produces an antibody fused to a phage coat protein.

18. The antibody library of claim 1, wherein the VH domain further comprises framework regions selected from the group consisting of: IGHJ4, IGHV1-46, IGHV1-69, IGHV3-15, and IGHV3-23.

19. The antibody library of claim 1, wherein the VL domain further comprises framework regions selected from the group consisting of: IGKV1-39, IGKV2-28, IGKV3-15, and IGKV4-1.

20. The antibody library of claim 1, wherein the plurality of antibodies comprises at least 7.6×10¹⁰ antibodies.

21. The antibody library of claim 1, wherein at least 95% of the plurality of antibodies are functional.

22. A method of preparing an antibody library, comprising:
a) obtaining sequence information for a plurality of VH-CDR3 and VL-CDR3 sequences from a pool of naïve B-cells and sequence information for a plurality of VH-CDR1, VH-CDR2, VH-CDR3, VL-CDR1, VL-CDR2, and VL-CDR3 sequences from a pool of memory B-cells;
b) assembling a plurality of variable light (VL) domain sequences, each VL domain sequence comprising: a VL-CDR1 sequence derived from the sequence information from memory B-cells determined in step a., a VL-CDR2 sequence derived from the sequence information from memory B-cells determined in step a., and a VL-CDR3 sequence derived from the sequence information from memory B-cells or naïve B-cells determined in step a.,
c) assembling a plurality of first nucleic acid sequences encoding a plurality of first antibodies, each first antibody comprising:
i. a variable light (VL) domain sequence assembled in step b.; and
ii. a single fixed heavy chain sequence;
d) inserting the plurality of first nucleic acid sequences into a plurality of phages;
e) expressing the plurality of first antibodies on the surface of the plurality of phages;
f) applying at least one selective pressure to the plurality of phages to produce a subset of phages comprising a subset of first nucleic acid sequences;
g) assembling a plurality of a variable heavy (VH) domain sequences, each VH domain sequence comprising: a VH-CDR1 sequence derived from the sequence information from memory B-cells determined in step a., a VH-CDR2 sequence derived from the sequence information from memory B-cells determined in step a., and a VH-CDR3 sequence derived from the sequence information from memory B-cells or naïve B-cells determined in step a., wherein at least one of the VH-CDR3 sequence and the VL-CDR3 sequence is derived from the sequence information from naïve B-cells;
h) replacing the single fixed heavy chain sequences from the subset of first nucleic acid sequences with the plurality of VH domain sequences assembled in step g. to produce a plurality of second nucleic acid sequences, each second nucleic acid sequence comprising:
i. a variable light (VL) domain sequence assembled in step b., and
ii. a variable heavy (VH) domain sequence assembled in step g.
wherein the plurality of second nucleic acid sequences encodes a plurality of second antibodies;
i) transforming a plurality of microbes with the plurality of phages to produce a plurality of transformants.

23-51. (canceled)

52. An antibody library that comprises a plurality of antibodies, wherein each antibody of the plurality of antibodies comprises:
a) a VH domain comprising a VH-CDR1 sequence, a VH-CDR2 sequence, and a VH-CDR3 sequence; and
b) a VL domain comprising a VL-CDR1 sequence, a VL-CDR2 sequence, and a VL-CDR3 sequence; wherein
c) a CDR sequence is selected from the group consisting of: a VH-CDR1 sequence, a VH-CDR2 sequence, a VH-CDR3 sequence, a VL-CDR1 sequence, a VL-CDR2 sequence, and a VL-CDR3 sequence, wherein the CDR sequence is the same for each antibody of the plurality of antibodies; and
d) a unique combination of remaining CDR sequences are selected from the group consisting of: a VH-CDR1 sequence, a VH-CDR2 sequence, a VH-CDR3 sequence, a VL-CDR1 sequence, a VL-CDR2 sequence, and a VL-CDR3 sequence.

53. The antibody library of claim 52, wherein the CDR sequence of (c) is a VH-CDR3 sequence.

54. The antibody library of claim 53, wherein remaining CDR sequences of (d) are a VH-CDR1 sequence, a VH-CDR2 sequence, a VL-CDR1 sequence, a VL-CDR2 sequence, and a VL-CDR3 sequence.

55. The antibody library of claim 52, wherein the CDR sequence of (c) is the same as a CDR sequence derived from an initial antibody clone.

56. The antibody library of claim 54, wherein each one of the remaining CDR sequences of (d) is present in the antibody library at a high degree of diversity.

57. The antibody library of claim 56, wherein the high degree of diversity comprises at least 1×10³ different CDR sequences.

58. (canceled)

59. The antibody library of claim 58, wherein the naturally occurring CDR sequence is derived from a human population.

60. The antibody library of claim 58, wherein the remaining CDR sequences of (d) are present in non-naturally occurring combinations for each antibody of the plurality of antibodies.

61. The antibody library of claim 55, wherein at least one antibody of the plurality of antibodies has at least one of the following: a higher melting temperature (Tm) as compared to an initial antibody clone, a higher affinity for a target epitope as compared to an initial antibody clone, or a higher cross-reactivity for a target epitope across two or more species as compared to an initial antibody clone.

62. The antibody library of claim 52, wherein at least one antibody of the plurality of antibodies has a melting temperature (Tm) that is from 50° C. to 90° C.

63. The antibody library of claim 52, wherein at least one antibody of the plurality of antibodies binds to a target epitope with a K_{d} of 100 nM or less.

64. A method for generating an antibody library, the method comprising:
(a) selecting a CDR sequence, wherein the CDR sequence is selected from the group consisting of: a VH-CDR1 sequence, a VH-CDR2 sequence, a VH-CDR3 sequence, a VL-CDR1 sequence, a VL-CDR2 sequence, and a VL-CDR3 sequence;
(b) replacing a CDR sequence for each antibody of a first antibody library with the CDR sequence selected in (a), thereby generating a second antibody library comprising a plurality of antibodies, wherein each antibody of the plurality of antibodies comprises:
(i) the CDR sequence selected in (a); and
(ii) a unique combination of remaining CDR sequences not selected in (a), wherein the remaining CDR sequences are selected from the group consisting of: a VH-CDR1 sequence, a VH-CDR sequence, a VH-CDR3 sequence, a VL-CDR1 sequence, a VL-CDR2 sequence, and a VL-CDR3 sequence.

65-79. (canceled)


SEQUENCE LISTING (99 sequences)

SEQ ID NO: 1 - protein, 32 aa, Artificial Sequence
  Description of Artificial Sequence Synthetic polypeptide
YTFSSYYLHGIINPSGGSTNYACARDPALDYW

SEQ ID NO: 2 - protein, 32 aa, Artificial Sequence
  Description of Artificial Sequence Synthetic polypeptide
YSFTDYYLHGGIIPIRGRAEYACARDHRRAYW

SEQ ID NO: 3 - protein, 33 aa, Artificial Sequence
  Description of Artificial Sequence Synthetic polypeptide
YSFTNYYIHGIINPSGGSAAYSCARVGPGLGYW

SEQ ID NO: 4 - protein, 33 aa, Artificial Sequence
  Description of Artificial Sequence Synthetic polypeptide
YSFTDYYLHGGIIPIRGRAEYACARGFGPLGYW

SEQ ID NO: 5 - protein, 34 aa, Artificial Sequence
  Description of Artificial Sequence Synthetic polypeptide
FILSTSGIHAAVSRSGGSTYYACARRRDDAFDIW

SEQ ID NO: 6 - protein, 34 aa, Artificial Sequence
  Description of Artificial Sequence Synthetic polypeptide
YTFTSYYMHGWISAYNGNTNYACARATTTDFDYW

SEQ ID NO: 7 - protein, 35 aa, Artificial Sequence
  Description of Artificial Sequence Synthetic polypeptide
GTFSYYAISGIINPSGDTTRYACARALPTGEVDYW

SEQ ID NO: 8 - protein, 35 aa, Artificial Sequence
  Description of Artificial Sequence Synthetic polypeptide
FILSTYGIHAAVSRSGGSTYYACARGRSSGYEDYW

SEQ ID NO: 9 - protein, 36 aa, Artificial Sequence
  Description of Artificial Sequence Synthetic polypeptide
YTFTSHIIHGWMNVYNGNRKYACARREGGVYGMDVW

SEQ ID NO: 10 - protein, 36 aa, Artificial Sequence
  Description of Artificial Sequence Synthetic polypeptide
YSFTDYYLHGGIIPIRGRAEYACARDPGVLYGLDVW

SEQ ID NO: 11 - protein, 37 aa, Artificial Sequence
  Description of Artificial Sequence Synthetic polypeptide
FTFSTSAMSSAISADGSWAGHACAKDSVAVGYGMDVW

SEQ ID NO: 12 - protein, 37 aa, Artificial Sequence
  Description of Artificial Sequence Synthetic polypeptide
YTFTTYYMHGWISAYNGNTNYACAREVPHFAGGMDVW

SEQ ID NO: 13 - protein, 38 aa, Artificial Sequence
  Description of Artificial Sequence Synthetic polypeptide
FILSTSGIHAAVSRSGGSTYYACARGERSYYYHYMDVW

SEQ ID NO: 14 - protein, 38 aa, Artificial Sequence
  Description of Artificial Sequence Synthetic polypeptide
YSFTDYYLHGGIIPIRGRAEYACAREGGSYQGTYFDYW

SEQ ID NO: 15 - protein, 38 aa, Artificial Sequence
  Description of Artificial Sequence Synthetic polypeptide
GTFTNYIIAGIINPSGGSTTYACARGSTYHQDTYFQHW

SEQ ID NO: 16 - protein, 39 aa, Artificial Sequence
  Description of Artificial Sequence Synthetic polypeptide
YSFTDYYLHGGIIPIRGRAEYACAKDRDQLLSSYGMDVW

SEQ ID NO: 17 - protein, 39 aa, Artificial Sequence
  Description of Artificial Sequence Synthetic polypeptide
YTFINYYMHGWMNPNSGNTGYACARGTPRGAAAGTFGYW

SEQ ID NO: 18 - protein, 40 aa, Artificial Sequence
  Description of Artificial Sequence Synthetic polypeptide
YTFTGHDMHGIIHPGGGTTSYACARRPPCSGGSCYPIDYW

SEQ ID NO: 19 - protein, 41 aa, Artificial Sequence
  Description of Artificial Sequence Synthetic polypeptide
FILSTSGIHAAVSRSGGSTYYACARDPWQWLVAVRGGMDVW

SEQ ID NO: 20 - protein, 41 aa, Artificial Sequence
  Description of Artificial Sequence Synthetic polypeptide
GTFSKYSISGIINTSGGSTSYACARAPGRYSGYWSDAFDIW

SEQ ID NO: 21 - protein, 42 aa, Artificial Sequence
  Description of Artificial Sequence Synthetic polypeptide
YTFTSYGISGMINPSGGSTSYACAKARERSGWYSNYYYMDVW

SEQ ID NO: 22 - protein, 43 aa, Artificial Sequence
  Description of Artificial Sequence Synthetic polypeptide
YTFTSYHMHGWMNPNSGNTGYACAKGWTYYYGSGSYPYYMDVW

SEQ ID NO: 23 - protein, 44 aa, Artificial Sequence
  Description of Artificial Sequence Synthetic polypeptide
GTFSSYAISGWINPSGGTTRYACARGGRSGGWYAGYYYYGMDVW

SEQ ID NO: 24 - protein, 44 aa, Artificial Sequence
  Description of Artificial Sequence Synthetic polypeptide
FILSTSGIHAAVSRSGGSTYYACARAYSSSWYIGGYYYYGMDVW

SEQ ID NO: 25 - protein, 45 aa, Artificial Sequence
  Description of Artificial Sequence Synthetic polypeptide
FTFSNYAMNSAISASGGRTYYACAKDVSYNYYGSGSAGYYGMDVW

SEQ ID NO: 26 - protein, 9 aa, Artificial Sequence
  Description of Artificial Sequence Synthetic peptide
HTFSDSYMH

SEQ ID NO: 27 - protein, 9 aa, Artificial Sequence
  Description of Artificial Sequence Synthetic peptide
GTFSSYAIS

SEQ ID NO: 28 - protein, 9 aa, Artificial Sequence
  Description of Artificial Sequence Synthetic peptide
YTFSSYAIS

SEQ ID NO: 29 - protein, 9 aa, Artificial Sequence
  Description of Artificial Sequence Synthetic peptide
YTFTGYYMH

SEQ ID NO: 30 - protein, 9 aa, Artificial Sequence
  Description of Artificial Sequence Synthetic peptide
YTFTSYFMH

SEQ ID NO: 31 - protein, 9 aa, Artificial Sequence
  Description of Artificial Sequence Synthetic peptide
YTFTDYYIH

SEQ ID NO: 32 - protein, 9 aa, Artificial Sequence
  Description of Artificial Sequence Synthetic peptide
YTFTDYYVH

SEQ ID NO: 33 - protein, 9 aa, Artificial Sequence
  Description of Artificial Sequence Synthetic peptide
HTFSDEYMH

SEQ ID NO: 34 - protein, 9 aa, Artificial Sequence
  Description of Artificial Sequence Synthetic peptide
YTFTSYGIS

SEQ ID NO: 35 - protein, 9 aa, Artificial Sequence
  Description of Artificial Sequence Synthetic peptide
YTFTSYYMH

SEQ ID NO: 36 - protein, 9 aa, Artificial Sequence
  Description of Artificial Sequence Synthetic peptide
YTFTTYYMH

SEQ ID NO: 37 - protein, 9 aa, Artificial Sequence
  Description of Artificial Sequence Synthetic peptide
GTFSNYAIS

SEQ ID NO: 38 - protein, 9 aa, Artificial Sequence
  Description of Artificial Sequence Synthetic peptide
YTFTNYYMH

SEQ ID NO: 39 - protein, 9 aa, Artificial Sequence
  Description of Artificial Sequence Synthetic peptide
YTFTDYYMH

SEQ ID NO: 40 - protein, 9 aa, Artificial Sequence
  Description of Artificial Sequence Synthetic peptide
YTFTSHYMH

SEQ ID NO: 41 - protein, 13 aa, Artificial Sequence
  Description of Artificial Sequence Synthetic peptide
GVINPSGDSTTYA

SEQ ID NO: 42 - protein, 13 aa, Artificial Sequence
  Description of Artificial Sequence Synthetic peptide
GIINPSGGSTSYA

SEQ ID NO: 43 - protein, 13 aa, Artificial Sequence
  Description of Artificial Sequence Synthetic peptide
GAINPSGSSTRYA

SEQ ID NO: 44 - protein, 13 aa, Artificial Sequence
  Description of Artificial Sequence Synthetic peptide
GGIIPIFGTANYA

SEQ ID NO: 45 - protein, 13 aa, Artificial Sequence
  Description of Artificial Sequence Synthetic peptide
GMITPSGGSTTYA

SEQ ID NO: 46 - protein, 13 aa, Artificial Sequence
  Description of Artificial Sequence Synthetic peptide
GIINPSGGSTTYA

SEQ ID NO: 47 - protein, 13 aa, Artificial Sequence
  Description of Artificial Sequence Synthetic peptide
GTINPSGATTNYA

SEQ ID NO: 48 - protein, 13 aa, Artificial Sequence
  Description of Artificial Sequence Synthetic peptide
GVINPSGGGTTYA

SEQ ID NO: 49 - protein, 13 aa, Artificial Sequence
  Description of Artificial Sequence Synthetic peptide
GVINPSGGSTSYA

SEQ ID NO: 50 - protein, 13 aa, Artificial Sequence
  Description of Artificial Sequence Synthetic peptide
GIINPSVGSTGYA

SEQ ID NO: 51 - protein, 13 aa, Artificial Sequence
  Description of Artificial Sequence Synthetic peptide
GWMNPNSGNTGYA

SEQ ID NO: 52 - protein, 12 aa, Artificial Sequence
  Description of Artificial Sequence Synthetic peptide
CARDQVGPSDYW

SEQ ID NO: 53 - protein, 9 aa, Artificial Sequence
  Description of Artificial Sequence Synthetic peptide
  MOD_RES (5)..(5): Ser or Asp
  MOD_RES (8)..(8): Met or Ile
YTFTXYYXH

SEQ ID NO: 54 - protein, 13 aa, Artificial Sequence
  Description of Artificial Sequence Synthetic peptide
  MOD_RES (8)..(8): Gly or Asp
GVINPSGXSTTYA

SEQ ID NO: 55 - protein, 98 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 56 - protein, 98 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 57 - protein, 98 aa, Artificial Sequence
  Description of Artificial Sequence Synthetic polypeptide
QVQLVQSGAEVKKPGASVKVSCKASGYTFTGYYMHWVRQAPGQGLEWMGRINPNSGGTNYAQKFQGRVTSTRDTSISTAYMELSRLRSDDTVVYYCAR

SEQ ID NO: 58 - protein, 98 aa, Artificial Sequence
  Description of Artificial Sequence Synthetic polypeptide
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 59 - protein, 98 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 60 - protein, 98 aa, Artificial Sequence
  Description of Artificial Sequence Synthetic polypeptide
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 61 - protein, 98 aa, Artificial Sequence
  Description of Artificial Sequence Synthetic polypeptide
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 62 - protein, 98 aa, Artificial Sequence
  Description of Artificial Sequence Synthetic polypeptide
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 63 - protein, 97 aa, Artificial Sequence
  Description of Artificial Sequence Synthetic polypeptide
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCA

SEQ ID NO: 64 - protein, 98 aa, Artificial Sequence
  Description of Artificial Sequence Synthetic polypeptide
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 65 - protein, 99 aa, Artificial Sequence
  Description of Artificial Sequence Synthetic polypeptide
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAH

SEQ ID NO: 66 - protein, 99 aa, Artificial Sequence
  Description of Artificial Sequence Synthetic polypeptide
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCAR

SEQ ID NO: 67 - protein, 99 aa, Artificial Sequence
  Description of Artificial Sequence Synthetic polypeptide
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCAR

SEQ ID NO: 68 - protein, 98 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 69 - protein, 98 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 70 - protein, 98 aa, Artificial Sequence
  Description of Artificial Sequence Synthetic polypeptide
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 71 - protein, 97 aa, Artificial Sequence
  Description of Artificial Sequence Synthetic polypeptide
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 72 - protein, 98 aa, Artificial Sequence
  Description of Artificial Sequence Synthetic polypeptide
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAR

SEQ ID NO: 73 - protein, 98 aa, Artificial Sequence
  Description of Artificial Sequence Synthetic polypeptide
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 74 - protein, 98 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 75 - protein, 98 aa, Artificial Sequence
  Description of Artificial Sequence Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAK

SEQ ID NO: 76 - protein, 100 aa, Artificial Sequence
  Description of Artificial Sequence Synthetic polypeptide
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 77 - protein, 98 aa, Artificial Sequence
  Description of Artificial Sequence Synthetic polypeptide
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 78 - protein, 100 aa, Artificial Sequence
  Description of Artificial Sequence Synthetic polypeptide
EVQLVESGGGLVQPGRSLRLSCTASGFTFGDYAMSWFRQAPGKGLEWVGFIRSKAYGGTTEYTASVKGRFTISRDGSKSIAYLQMNSLKTEDTAVYYCTR

SEQ ID NO: 79 - protein, 100 aa, Artificial Sequence
  Description of Artificial Sequence Synthetic polypeptide
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 80 - protein, 98 aa, Artificial Sequence
  Description of Artificial Sequence Synthetic polypeptide
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCAR

SEQ ID NO: 81 - protein, 100 aa, Artificial Sequence
  Description of Artificial Sequence Synthetic polypeptide
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 82 - protein, 97 aa, Artificial Sequence
  Description of Artificial Sequence Synthetic polypeptide
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 83 - protein, 98 aa, Artificial Sequence
  Description of Artificial Sequence Synthetic polypeptide
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAK

SEQ ID NO: 84 - protein, 98 aa, Artificial Sequence
  Description of Artificial Sequence Synthetic polypeptide
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 85 - protein, 96 aa, Artificial Sequence
  Description of Artificial Sequence Synthetic polypeptide
EVQLVESRGVLVQPGGSLRLSCAASGFTVSSNEMSWVRQAPGKGLEWVSSISGGSTYYADSRKGRFTISRDNSKNTLHLQMNSLRAEDTAVYYCKK

SEQ ID NO: 86 - protein, 99 aa, Artificial Sequence
  Description of Artificial Sequence Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 87 - protein, 97 aa, Artificial Sequence
  Description of Artificial Sequence Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 88 - protein, 99 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 89 - protein, 97 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 90 - protein, 99 aa, Artificial Sequence
  Description of Artificial Sequence Synthetic polypeptide
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 91 - protein, 98 aa, Artificial Sequence
  Description of Artificial Sequence Synthetic polypeptide
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYCCAR

SEQ ID NO: 92 - protein, 99 aa, Artificial Sequence
  Description of Artificial Sequence Synthetic polypeptide
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCAR

SEQ ID NO: 93 - protein, 98 aa, Artificial Sequence
  Description of Artificial Sequence Synthetic polypeptide
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAR

SEQ ID NO: 94 - protein, 98 aa, Artificial Sequence
  Description of Artificial Sequence Synthetic polypeptide
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 95 - protein, 98 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 96 - protein, 98 aa, Artificial Sequence
  Description of Artificial Sequence Synthetic polypeptide
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 97 - protein, 101 aa, Artificial Sequence
  Description of Artificial Sequence Synthetic polypeptide
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 98 - protein, 98 aa, Artificial Sequence
  Description of Artificial Sequence Synthetic polypeptide
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCAR

SEQ ID NO: 99 - protein, 98 aa, Artificial Sequence
  Description of Artificial Sequence Synthetic polypeptide
QVQLVQSGAEVKKPGASVKVSCKASGYTFTSYAMHWVRQAPGQRLEWMGWSNAGNGNTKYSQEFQGRVTITRDTSASTAYMELSSLRSEDMAVYYCAR